(19) United States
(12) Patent Application Publication
(10) Pub. No.: US 2026/0097122 A1
(43) Pub. Date: Apr. 9, 2026

(54) CD19/C22 CAR T-CELL TREATMENT OF HIGH RISK OR RELAPSED PEDIATRIC ACUTE LYMPHOBLASTIC LEUKEMIA

(71) Applicant: AUTOLUS LIMITED, london (GB)

(72) Inventors: Martin Pulé, London (GB); Persis Amrolia, London (GB); Sara Ghorashian, London (GB)

(21) Appl. No.: 18/862,269

(22) PCT Filed: May 10, 2023
(86) PCT No.: PCT/IB2023/054844

§ 371 Date: Nov. 1, 2024

Related U.S. Application Data
(60) Provisional application No. 63/340,857, filed on May 11, 2022.
(60) Provisional application No. 63/350,861, filed on Jun. 9, 2022.
(60) Provisional application No. 63/386,742, filed on Dec. 9, 2022.
(60) Provisional application No. 63/498,458, filed on Apr. 26, 2023.

(51) Int. Cl. A61K 40/42; A61K 40/11; A61K 40/31; A61P 35/02

(52) U.S. Cl. CPC A61K 40/4211; A61K 40/11; A61K 40/31; A61K 40/4212; A61P 35/02; A61K 2239/13; A61K 2239/28; A61K 2239/48

(57) ABSTRACT

The present disclosure relates to CD19/22 CAR T-cell products and methods for treating high risk or relapsed CD19+ or CD22+ haematological malignancies.

### CROSS-REFERENCE TO RELATED APPLICATIONS

This application claims the benefit of U.S. provisional application No. 63/340,857 filed 11 May 2022, U.S. provisional application No. 63/350,861 filed 9 Jun. 2022, U.S. provisional application No. 63/386,742 filed 9 Dec. 2022, and U.S. provisional application No. 63/498,458 filed 26 Apr. 2023. These applications are incorporated herein by reference in their entirety.

### FIELD

The disclosure relates to CD19/22 CAR T-cell products and methods for treating high risk or relapsed, CD19+ or CD22+ haematological malignancies.

### INCORPORATION BY REFERENCE OF THE SEQUENCE LISTING

This application contains, as a separate part of disclosure, a Sequence Listing in computer-readable form (Filename: 57768_SeqListing.txt; 121,399 bytes—XML file dated Dec. 8, 2022) which is incorporated by reference herein in its entirety.

### BACKGROUND

Pediatric B-cell acute lymphoblastic leukemia (ALL) accounts for about 30% of childhood cancer diagnoses. It is a serious and life-threatening disease and will progress rapidly if left untreated. B-ALL is characterized by the rapid proliferation of poorly differentiated lymphoid progenitor cells inside the bone marrow. Standard of care is combination chemotherapy. Typically, treatment procedures are divided into several phases: steroid pre-phase, induction, consolidation, intensification, and maintenance, and involve the administration of steroids, chemotherapy, targeted carcer drugs and/or bone marrow or stem cell transplant (SCT). The overall survival (OS) rate for pediatric B cell ALL patients is 90%, however 10-20% of pediatric B cell ALL patients relapse with chemoresistant disease. The clinical outcomes for relapsing pediatric patients have not changed over the past two decades. The long-term OS of children suffering a relapse of ALL remains 40-50% despite considerable effort to optimize standard approaches. Current treatment strategies for relapsed ALL include either further intensive chemotherapy and often consolidation with allogeneic SCT, but these patients have poor outcomes. Since most of these patients have frequently had the maximal tolerable dose of chemo/radiotherapy, novel therapies are urgently required for such patients.

A number of immunotherapeutic agents have been described for use in cancer treatment, including therapeutic monoclonal antibodies (mAbs), immunoconjugated mAbs, radioconjugated mAbs and bi-specific T-cell engagers. Typically these immunotherapeutic agents target a single antigen: for instance, Rituximab targets CD20; Myelotarg targets CD33; and Alemtuzumab targets CD52.

Chimeric antigen receptors are proteins which graft the specificity of a monoclonal antibody (mAb) to the effector function of a T-cell. Their usual form is that of a type I transmembrane domain protein with an antigen recognizing amino terminus (binder), and a transmembrane domain connected to an endodomain which transmits T-cell activation signals. The most common form of these molecules are fusions of single-chain variable fragments (scFv) derived from monoclonal antibodies, which recognize a target antigen, fused via a trans-membrane domain to a signalling endodomain. Such molecules result in activation of the T-cell in response to recognition by the scFv of its target. When T cells express such a CAR, they recognize and kill target cells that express the target antigen. CARs have been developed against various tumor-associated antigens and many are currently undergoing clinical trials.

The human CD19 antigen is a 95 kd transmembrane glycoprotein belonging to the immunoglobulin superfamily. CD19 is expressed very early in B-cell differentiation and is only lost at terminal B-cell differentiation into plasma cells. Consequently, CD19 is expressed on all B-cell malignancies apart from multiple myeloma. Since loss of the normal B-cell compartment is an acceptable toxicity, CD19 has been a CAR target and clinical studies targeting CD19 with CARs have been conducted.

CD19-directed CAR therapy has shown efficacy in treating ALL. The first studies in ALL were published in Spring 2013, by groups from Memorial Sloane Kettering [Brentjens et al., Leukemia. Sci. Transl. Med., 5: 177ra38)(2013)] and the University of Pennsylvania. An updated report of the University of Pennsylvania study was made [Maude et al., N. Engl. J. Med., 371: 1507-1517 (2014)]. In that latter study, twenty-five patients under the age of 25 years and five over that age were treated. 90% achieved a complete response at one month, 22 of 28 evaluable cases achieved a minimal residual disease (MRD) negative status and the 6 month event free survival rate was 67%. Fifteen patients received no further therapy after the study.

The Memorial Sloane Kettering study was in the adult setting, and treated five ALL patients (two with refractory relapse, two with MRD positive disease and one who was MRD negative) with autologous T cells retrovirally transduced to express a CD19 CAR incorporating an scFv derived from the SJ25C1 hybridoma and a CD28 co-stimulatory domain. All of these patients achieved a deep molecular remission, enabling four of these patients to receive an allogeneic SCT. This precluded assessment of the durability of responses, but CAR T cells were only detectable in the blood or bone marrow for 3-8 weeks after infusion. The patient who was not transplanted relapsed at 90 days with CD19+ disease. Subsequently, Davila et al., Sci. Transl. Med. 6: 224:ra25 (2014) provided an update of this cohort 14 of 16 adult patients had detectable disease at the point of CAR T cell infusion, despite salvage chemotherapy and cyclophosphamide conditioning. 14 of 16 achieved a complete remission with or without count recovery including 7 of 9 patients with morphologic evidence of residual disease detectable after salvage chemotherapy. 12 of 16 patients achieved MRD negativity and this allowed seven to undergo allogeneic transplantation by the time of publication. Responses were durable in some patients with 4 of 8 non-transplanted patients continuing in morphological remission at up to 24 months follow-up although the survival curves for this cohort are not yet stable.

Another published study of a cohort of pediatric and young adult patients predominantly with ALL provides the first intention-to-treat analysis of its outcomes. This may help remove the bias inherent in excluding patients who do not receive the anticipated dose of CAR T cells [Lee et al., Lancet (2014) doi:10.1016/S0140-6736(14)61403-3]. Twenty-one patients were treated with a CD28 domain-containing second generation CAR. All but two patients received the anticipated T cell dose, highlighting the feasibility of delivering this treatment to those with refractory or multiply-relapsed ALL. This study showed 67% achieving a complete remission and 60% of those with ALL achieving MRD negative status.

The first trial showing clinical activity of CD22 CAR T cells in children and adults with B-ALL was reported in Fry et al., Nature Med., 24:20-28 (2018). 21 children and adults, including 17 who were previously treated with CD19-directed immunotherapy, received CD22 CAR T cells. Complete remission obtained in 73% (11/15) of patients receiving ≥1×10⁶ CD22-CAR T cells per kg body weight, including 5 of 5 patients who enrolled with CD19 dim/neg relapse. Median remission duration was 6 months. Relapses were associated with diminished CD22 site density that likely permitted CD22+ cell escape from killing by CD22-CAR T cells.

A particular problem in the field of oncology is provided by the Goldie-Coldman hypothesis: which describes that the sole targeting of a single antigen may result in tumor escape by modulation of said antigen due to the high mutation rate inherent in most cancers. This modulation of antigen expression may reduce the efficacy of known immunotherapeutics, including those which target CD19. Despite the excellent clinical responses to CD19-directed T cell therapies, a significant number of patients still relapse. The major cause of disease relapse is either the emergence of CD19 negative leukaemic clones or non-persistence of the CAR T cells [Sotillo et al., Cancer Discov., 5:1282-1295 (2015); Gardner et al., Blood 127:2406-2410 (2016)].

Thus a problem with immunotherapeutics targeted against CD19 is that a B-cell malignancy may mutate and become CD19-negative. This may result in relapse with CD19-negative cancers which are not responsive to CD19 targeted therapeutics. The emergence of CD19 negative escape clones has been reported in all the major studies in ALL and may relate to selection of leukaemic clones with either somatic mutations or expressing an alternatively spliced CD19 mRNA lacking exon 2 that prevent recognition by the CD19 CAR (Sotillo et al., supra). In the paediatric B cell ALL studies at the University of Pennsylvania, two-thirds of patients relapsed due to CD19 negative disease, while the remaining one-third relapsed due to poor CAR T cell engraftment [Grupp et al., Blood 128(22):221 (2016)]. CD19 negative relapse has also been reported by NCI in a lymphoma patient treated with fully human anti-CD19 CAR (HuCAR-19) [Brudno et al., Blood, 128(22): 999(2016)]. Another study has recently reported that CD19-negative relapses were more frequently observed, post Tisagenlecleucel infusion, in patients with high tumor burden [Dourthe et al., Leukaemia, 35:3383-3393 (2021)].

There is thus a need for immunotherapeutic agents which are capable of targeting more than one cell surface structure to reflect the complex pattern of marker expression that is associated with many cancers, including CD19-positive cancers.

Although CAR-T cell-mediated treatment has shown success towards compact target antigens such as CD19 or GD2, chimeric antigen receptors have failed to signal in response to antigens with bulky extracellular domains.

An optimum synaptic distance is required for efficient triggering of downstream signaling after antigen encounter. Upon T cell encounter with an antigen presenting cell (via TCR interaction with peptide MHC), proteins at the interface segregate passively based on size. Phosphatases such as CD45 and CD148, which have large ectodomains, are excluded from regions of close contact between the T cell and APC. The synapse formed through interaction of peptide MHC and TCR is optimal for occlusion of CD45. In the case of CAR-T cells targeting smaller antigens such as CD19, there is no barrier to synapse formation and such antigens can be targeted efficiently at multiple epitopes. Large proteins such as CD22 pose a unique problem. Targeting a membrane distal epitope on such proteins is likely to provide a suboptimal synapse length allowing phosphatases to enter the synapse and inhibit tyrosine phosphorylation. Targeting membrane proximal regions may improve synapse formation, however steric occlusion of the epitope is likely to lead to suboptimal ligation of the target allowing the presence of phosphatases within the synapse, dampening tyrosine phosphorylation, kinase activity and thus CAR signaling.

There is therefore a need for alternative CAR T-cell approaches, capable of killing target cells expressing a large or bulky target antigen.

T cell exhaustion is a state of T cell dysfunction that arises during many chronic infections and cancer. It is defined by poor effector function, sustained expression of inhibitory receptors and a transcriptional state distinct from that of functional effector or memory T cells. Exhaustion prevents optimal control of infection and tumors. Recently, a clearer picture of the functional and phenotypic profile of exhausted T cells has emerged with expression of inhibitory receptor programmed death 1 (PD-1; also known as PDCD1), a negative regulator of activated T cells, being a key feature [Day et al., Nature, 443 350-354 (2006)].

Responses in CD19 CAR studies suggest that persistence of T-cells for a protracted period at high levels seems to be important in effecting durable responses [Mueller et al., Blood, 130, 2317-2325 (2017)].

There remains a need in the art for effective CAR therapies for CD19+ or CD22+ haematological malignancies which are not associated with the foregoing disadvantages.

The disclosure provides methods for treating high risk/relapsed CD19+ or CD22+ haematological malignancy in a patient comprising administering to the patient autologous CD19/22 CAR T-cells (for example, the autologous CD19/22 CAR T-cell product comprising CAT19CAR and 9A8CAR CARs described in Example 1 herein). The disclosure also provides autologous CD19/22 CAR T-cells (for example, the autologous CD19/22 CAR T-cell product comprising CAT19CAR and 9A8CAR CARs described in Example 1 herein), or a pharmaceutical composition comprising these cells, for use in the treatment of high risk/relapsed CD19+ or CD22+ haematological malignancy. The disclosure also provides the use of autologous CD19/22 CAR T-cells (for example, the autologous CD19/22 CAR T-cell product comprising CAT19CAR and 9A8CAR CARs described in Example 1 herein), or a pharmaceutical composition comprising these cells, in the manufacture of a medicament for the treatment of high risk/relapsed CD19+ or CD22+ haematological malignancy.

Methods, autologous CD19/22 CAR T-cells, or uses are provided wherein the age of the patient is twenty-four years or younger.

Methods, autologous CD19/22 CAR T-cells, or uses are provided wherein the haematological malignancy is acute lymphoblastic leukemia (ALL), or a CD19+ or CD22+ lymphoma. Methods are provided wherein the lymphoma is Burkitt lymphoma.

Methods, autologous CD19/22 CAR T-cells, or uses are provided in particular wherein the patient has: a) resistant disease (>5% blasts) at end of UKALL 2019 guidelines or equivalent induction, b) ALL with persisting high level MRD at 2^{nd} time point of frontline national protocol (currently MRD>10⁻⁴ at week 14 UKALL2019 guidelines or equivalent), c) high risk infant ALL (age <6 months at diagnosis with MLL gene rearrangement and either presenting white cell count >300×10⁹/L or poor steroid early response (i.e. circulating blast count >1×10⁹/L following 7 day steroid pre-phase of induction as per national guidelines or equivalent), d) intermediate risk infant ALL with MRD>10⁻³ at end of induction following national guidelines or equivalent), e) high risk first relapse (as defined by updated IntreALL 2019 classification: bone marrow or combined relapse within thirty months of diagnosis), f) standard risk relapse in patients with high risk cytogenetics (defined as BCR-ABL, KMT2A rearrangement, near-haploidy (<30 chromosomes) and low hypodiploidy (30-39 chromosomes), iAMP21 and TCF3-HLF translocations), g) standard risk relapse with bone marrow minimal residual disease (MRD)>10⁻³ at end of re-induction, h) any refractory relapse of ALL (defined as >1% blasts by flow cytometry after at least one cycle of standard chemotherapy), or i) any relapse of CD22+ lymphoma. The patient may have an isolated CNS relapse meeting one or more of a)-o).

In the methods, autologous CD19/22 CAR T-cells, or uses provided, the patient is administered a single dose of 0.75×10⁶ CAR T-cells/kg, 1×10⁶ CAR T-cells/kg or 1.2×10⁶ CAR T-cells/kg body weight. The administration may be an intravenous injection, preferably through a Hickman line or peripherally inserted central catheter.

Methods, autologous CD19/22 CAR T-cells, or uses are provided wherein the CD19/22 CAR T-cells express a chimeric antigen receptor (CAR) comprising a CD19-binding domain which comprises

a) a heavy chain variable region (VH) having
complementarity determining regions (CDRs)
with the following sequences:
CDR1-
(SEQ ID NO: 1)
GYAFSSS;
CDR2-
(SEQ ID NO: 2)
YPGDED
CDR3-
(SEQ ID NO: 3)
SLLYGDYLDY;
and
b) a light chain variable region (VL) having
CDRs with the following sequences:
CDR1-
(SEQ ID NO: 4)
SASSSVSYMH;
CDR2-
(SEQ ID NO: 5)
DTSKLAS
CDR3-
(SEQ ID NO: 6)
QQWNINPLT.

The CDRs may be grafted on to a human antibody framework.

In provided methods, autologous CD19/22 CAR T-cells, or uses, the CD19-binding domain may comprise a VH domain having the sequence shown as SEQ ID NO: 7 and/or or a VL domain having the sequence shown as SEQ ID NO: 8, or a variant of either thereof having at least 95% sequence identity.

The CD19-binding domain may comprise an scFv in the orientation VH-VL. The CD19-binding domain may comprise the sequence shown as SEQ ID NO: 9 or a variant thereof having at least 90% sequence identity. The CD19-binding domain and a transmembrane domain may be connected in the CAR by a spacer such as a CD8 stalk. The CAR may comprise intracellular T cell signaling domain such as an intracellular T-cell signaling domain comprising the 41BB endodomain and the CD3-Zeta endodomain.

Methods, autologous CD19/22 CAR T-cells, or uses are provided wherein the CD19/22 CAR T-cells express a CAR comprising a CD22-binding domain which comprises:

a) a heavy chain variable region (VH) having
CDRs with the following sequences:
CDR1-
(SEQ ID NO: 58)
NFAMA;
CDR2-
(SEQ ID NO: 59)
SISTGGGNTYYRDSVKG
CDR3-
(SEQ ID NO: 60)
QRNYYDGSYDYEGYTMDA;
and
b) a light chain variable region (VL) having
CDRs with the following sequences:
CDR1-
(SEQ ID NO: 61)
RSSQDIGNYLT;
CDR2-
(SEQ ID NO: 62)
GAIKLED
CDR3-
(SEQ ID NO: 63)
LQSIQYP.

The CDRs may be grafted on to a human antibody framework.

In provided methods, autologous CD19/22 CAR T-cells, or uses, the CD22-binding domain may comprise a VH domain having the sequence shown as SEQ ID NO: 64 and/or or a VL domain having the sequence shown as SEQ ID NO: 65 or a variant thereof having at least 95% sequence identity.

The CD22-binding domain may comprise an scFv in the orientation VH-VL. The CD22-binding domain may comprise the sequence shown as SEQ ID NO: 966 or a variant thereof having at least 90% sequence identity. The CD22-binding domain and a transmembrane domain may be connected in the CAR by a spacer such as a CD8 stalk. The CAR may comprise intracellular T cell signaling domain such as an intracellular T-cell signaling domain comprising the 41BB endodomain and the CD3-Zeta endodomain.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1. A) Cartoons of CD19 CATCAR (which is AUTO1)(left) and CD22 9A8CAR (right). These CARs are type I transmembrane proteins. Both CARs are identical except for the scFv. The scFv are at the amino-terminus are linked to a CD8 stalk and transmembrane domain which is linked to an endodomain comprised of a fusion between 4-1BB and CD34. B) Dual lentiviral vectors encoding CATCAR (top) and 9A8CAR (bottom).

FIG. 2. CD19/22 CAR T cells retain their ability to kill single positive and low density CD22 targets. Non-transduced (NT), AUTO1 transduced, 9A8 (CD22 9A8CAR) transduced and AUTO1/22 (CAT/9A8 CAR) co-transduced T-cells were co-cultured 1:8 with A) Ligand negative target (SupT1 NT); B) CD19 CD22 positive targets (SupT1 CD19/CD22); C) CD19 positive targets (SupT1 CD19); D) High density CD22 targets (SupT1 CD22 high); and E) Low density CD22 targets (SupT1 CD22 low). Killing was determined by flow-cytometry after 72 hours.

FIG. 3. AUTO1/22 CAR T cells maintain cytolytic activity with CD19 antigen escape targets. Target cells: A) Raji; B) Raji with CD19 knock-out; C) CD19+/CD22+ primary B-ALL target cell; D) CD19neg/CD22+ primary B-ALL target cell.

FIG. 4. AUTO1/22 CAR T cells enhanced activity in vivo. A) Schedule of the in vivo model. B) and C) NSG mice were intravenously injected with 1×10⁶ Nalm-6 WT (B) or Nalm-6 CD19KO (neg) cells (C), and treated with non-transduced (NT), AUTO1 or AUTO1/22 normal donor T cells. Serial bioluminescence imaging was taken. All cells expressed HA-tagged firefly Luciferase. D) and E) Spider plot showing bioluminescence flux for each mouse over time; Nalm-6 WT (D) or Nalm-6 CD19KO (neg) cells (E). F) In vivo expansion of the different CAR T cell populations. Differential engraftment of different CAR T cell populations. The proportion of the different subpopulation of CAR T cells was determined by staining for AUTO1 with anti-idiotype and 9A8CAR with soluble recombinant CD22 ectodomain. The proportion of CAR double-negative, single-positive and double-positive T cells are shown for the product (before injection) and from bone marrow aspirated from mice at day 14 after injection into mice burdened with either NALM6 cells or NALM6 CD19 ko cells.

FIG. 5. Expression of the CD19 CAR and/or 22 CAR transgene in scale-up T cell clinical product. CAR T-cell populations were identified by pre-gating on live, CD45+/CD3+ cells. CAR-expressing cells were then identified by binding of anti-idiotype antibodies specific for the CD19 and CD22 CARs. A) Flow cytometry of two exemplary cell products. B) Histograms showing the proportions of CD19 CAR and CD22 single CAR populations, and dual CD19/CD22 CAR transduced population in the Advanced Therapy Investigational Medicinal Product (ATIMP)(n=11). C) Representative product memory immunophenotype. D) Summary of median memory subsets of all products. AUTO1/22 memory phenotype composition, Tcm=T central memory (CD45RA-CD62L+), Tn/TSCM=T naïve/stem cell like memory T cells (CD45RA+CD62L+), TEMRA=terminally differentiated memory (CD45RA+CD62L-), TEM=effector memory (CD45RA-CD62L-).

FIG. 6. Early CD19/22 CAR T cell persistence in patients.

FIG. 7. Annotated DNA sequence (SEQ ID NO: 88) encoding the CD19 CATCAR (AUTO 1).

FIG. 8. Annotated amino acid sequence (SEQ ID NO: 89) of the CD19 CATCAR (AUTO 1).

FIG. 9. Annotated DNA sequence (SEQ ID NO: 90) encoding the CD22 9A8CAR.

FIG. 10. Annotated amino acid sequence (SEQ ID NO: 91) of the CD22 9A8CAR.

FIG. 11. CAR-T cell expansion and persistence in the patients. Transgene-specific sequences for the CD19 and CD22 CAR were detected by qPCR in peripheral blood samples taken on days 0, 2, 7, 14 and 28, monthly up to 6 months, 6 weekly to 1 year then 3 monthly up to 18 months post infusion. The validated threshold for detection was 100 copies/ug DNA.Copies of A) CD19 CAR and B) CD22 CAR per microgram DNA were determined by qPCR in blood samples at different time points after CAR-T cell infusion. C) Expression of CD19 CAR and CD22 CAR in CAR-T cells were detected by flow cytometry in blood samples at different time points post infusion in an exemplary patient.

FIG. 12. Swimmers plot of all patients (n=12).

FIG. 13. Consort diagram all patients (n=12).

FIG. 14. Survival curves of all patients (n=12).

### DETAILED DESCRIPTION

In the CARPALL clinical study [Ghorashian et al., J. Haematol., 169. 463-478 (2015); Ghorashian et al., Nature Med., 25:1408-14 (2019)] which tested the efficacy of CD19 CATCAR (sometimes also referred to as CATi9CAR or AUTO1 herein), an autologous fast off-rate CAR, in children and young adults with relapsed/refractory (r/r) B-ALL, AUTO1 was efficacious with molecular remission in twelve of fourteen patients. However, of the seven patients that relapsed, five relapses were due to CD19 antigen negative escape. To address antigen escape relapse in AUTO1, an additional ligand could be targeted.

CD22 is contemplated herein as another target for B-ALL malignancies, although generating an effective CAR to CD22 is challenging due to the size, density and rigidity of this ligand. Furthermore, in the B-ALL setting, CD22 expression levels are known to down regulate in response to selective CAR pressure. In a clinical trial of the CD22 CAR2, this resulted in patients relapsing with lower CD22 density post-treatment (2,839 epitopes/cell) presumably due to the target density falling below the sensitivity threshold for the CD22 CAR2 (Fry et al., supra)].

The methods provided herein improve the treatment of r/r B-ALL by combining a highly sensitive CD22 CAR capable of targeting cells that express less than one thousand CD22 molecules per cell with AUTO1 to generate a dual targeting CD19 and CD22 product (AUTO1/22) via co-transduction for the treatment of pediatric r/r B-ALL. Results described in the Examples below were obtained as part of an extension cohort of the CARPALL clinical trial (NCT02443831).

### Chimeric Antigen Receptors (CARs)

A classical chimeric antigen receptor (CAR) is a chimeric type I trans-membrane protein which connects an extracellular antigen-binding domain to an intracellular signalling domain (endodomain). The antigen-binding domain is typically a single-chain variable fragment (scFv) derived from a monoclonal antibody (mAb), but it can be based on other formats which comprise an antibody fragment or an antibody-like antigen-binding site. Other examples include, but are not limited to: a natural ligand of the target antigen, a peptide with sufficient affinity for the target, a F(ab) fragment, a F(ab′)2 fragment, a F(ab′) fragment, a single domain antibody (sdAb), a domain antibody (dAb), a VHH antigen-binding domain or nanobody, an artificial single binder such as a DARPin (designed ankyrin repeat protein), an affibody, a fibronectin artificial antibody scaffold, an anticalin, an affilin, a VNAR, an iBody, an affimer, a fynomer, an abdurin/nanoantibody, a centyrin, an alphabody, a nanofitin, or a single-chain derived from a T-cell receptor which is capable of binding the target antigen. A spacer is usually necessary to isolate the antigen-binding domain from the membrane and to allow it a suitable orientation. A common spacer used is the Fc of IgG1. More compact spacers can suffice, e.g., the stalk from CD8a and even just the IgG1 hinge alone, depending on the antigen. A transmembrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. One common co-stimulatory domain is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

When the CAR binds the target antigen, an activating signal is transmitted to the T-cell on which the CAR is expressed thereby directing the specificity and cytotoxicity of the T cell towards cells expressing the target antigen.

### Target Antigens

A ‘target antigen’ is an entity which is specifically recognized and bound by the antigen-binding domains of a chimeric receptor provided herein.

The target antigen may be an antigen present on a cancer cell, for example, a tumor-associated antigen. CD19 and CD22 are target antigens contemplated herein.

### Binding Domains Specific for CD19 Target Antigen

The human CD19 antigen is a 95 kd transmembrane glycoprotein belonging to the immunoglobulin superfamily. CD19 is classified as a type I transmembrane protein, with a single transmembrane domain, a cytoplasmic C-terminus, and extracellular N-terminus. CD19 is expressed very early in B-cell differentiation and is only lost at terminal B-cell differentiation into plasma cells. CD19 is a biomarker for normal B cells as well as follicular dendritic cells. CD19 primarily acts as a B cell co-receptor in conjunction with CD21 and CD81. Upon activation, the cytoplasmic tail of CD19 becomes phosphorylated, which leads to binding by Src-family kinases and recruitment of PI-3 kinase.

CD19 is also expressed on all B-cell malignancies but not multiple myeloma cells. It is not expressed on other haematopoietic populations or non-haematopoietic cells and therefore targeting this antigen should not lead to toxicity to the bone marrow or non-haematopoietic organs. Loss of the normal B-cell compartment is considered an acceptable toxicity when treating lymphoid malignancies, because although effective CD19 CAR T cell therapy will result in B cell aplasia, the consequent hypogammaglobulinemia can be treated with pooled immunoglobulin.

Different designs of CARs have been tested against CD19 in various clinical trials, as outlined in the following Table 1.

TABLE 1

| Center | Binder | Endodomain | Comment |
|---|---|---|---|
| University College | Fmc63 | CD3-Zeta | Low-level brief |
| London | | | persistence |
| Memorial Sloane | SJ25C1 | CD28-Zeta | Short-term persistence |
| Kettering | | | |
| NCI/KITE | Fmc63 | CD28-Zeta | Long-term low-level |
| | | | persistence |
| Baylor, Centre for Cell | Fmc63 | CD3-Zeta/ | Short-term low-level |
| and Gene Therapy | | CD28-Zeta | persistence |
| UPENN/Novartis | Fmc63 | 41BB-Zeta | Long-term high-level |
| | | | persistence |

As shown above, most of the studies conducted to date have used an scFv derived from the hybridoma fmc63 as part of the binding domain to recognize CD19.

The antigen-binding domain of a CAR which binds to CD19 (referred to as a CD19 CAR herein) may be any domain which is capable of binding CD19.

For example, the antigen-binding domain may comprise a CD19 antigen-binding domain as described in Table 2.

TABLE 2

| Antigen-binding | |
|---|---|
| domain | Documents |
| HD63 | Pezzutto et al., J. Immunol. Baltim. Md 1950, 138: |
| | 2793-2799 (1987) |
| 4g7 | Meeker et al., Hybridoma, 3: 305-320 (1984) |
| Fmc63 | Nicholson et al., Mol. Immunol., 34: 1157-1165 (1997) |
| B43 | Bejcek et al., Cancer Res., 55: 2346-2351 (1995) |
| SJ25C1 | Bejcek et al., supra |
| BLY3 | Bejcek et al., supra |
| B4, or | Roguska et al., Protein Eng., 9: 895-904 (1996) |
| re-surfaced, or | |
| humanized B4 | |
| HB12b, | Kansas and Tedder, Immunol. Baltim. Md 1950, 147: |
| optimized and | 4094-4102 (1991); Yazawa et al., Proc. Natl. Acad. |
| humanized | Sci. U. S. A., 102: 15178-15183 (2005); Herbst et al., |
| | J. Pharmacol. Exp. Ther., 335: 213-222 (2010) |

The gene encoding CD19 comprises ten exons: exons 1 to 4 encode the extracellular domain; exon 5 encodes the transmembrane domain; and exons 6 to 10 encode the cytoplasmic domain. The antigen-binding domain of a CD19 CAR herein may bind an epitope of CD19 encoded by exon 1 of the CD19 gene. The antigen-binding domain of a CD19 CAR herein may bind an epitope of CD19 encoded by exon 2 of the CD19 gene. The antigen-binding domain of a CD19 CAR herein may bind an epitope of CD19 encoded by exon 3 of the CD19 gene. The antigen-binding domain of a CD19 CAR herein may bind an epitope of CD19 encoded by exon 4 of the CD19 gene.

A CD19-binding domain exemplified herein comprises variable regions with complementarity determining regions (CDRs) from an antibody referred to as CAT19,

a) a heavy chain variable region (VH) having
CAT19 CDRs with the following sequences:
CDR1-
(SEQ ID NO: 1)
GYAFSSS;
CDR2-
(SEQ ID NO: 2)
YPGDED
CDR3-
(SEQ ID NO: 3)
SLLYGDYLDY;
and
b) a light chain variable region (VL) having
CAT19 CDRs with the following sequences:
CDR1-
(SEQ ID NO: 4)
SASSSVSYMH;
CDR2-
(SEQ ID NO: 5)
DTSKLAS
CDR3-
(SEQ ID NO: 6)
QQWNINPLT.

The CAT19 antibody is described in WO2016/139487.

It is contemplated that one or more mutations (substitutions, additions or deletions) can be introduced into one or more CDRs without negatively affecting CD19-binding activity. Each CDR may, for example, have one, two or three amino acid mutations.

The CDRs may be in the format of a single-chain variable fragment (scFv), which is a fusion protein of the heavy variable region (VH) and light chain variable region (VL) of an antibody, connected with a short linker peptide of ten to about 25 amino acids. The scFv may be in the orientation VH-VL, i.e., the VH is at the amino-terminus of the CAR molecule and the VL domain is linked to the spacer and, in turn the transmembrane domain and endodomain.

The CDRs may be grafted on to the framework of a human antibody or scFv. For example, the CAR may comprise a CD19-binding domain consisting or comprising one of the following sequences.

The CD19 CAR may comprise the following VH sequence.
SEQ ID NO: 7
VH sequence from CAT19 murine monoclonal antibody
QVQLQQSGPELVKPGASVKISCKASGYAFSSSWMNWVKQRPGKGLEWIGRIYPGDEDTN
YSGKFKDKATLTADKSSTTAYMQLSSLTSEDSAVYFCARSLLYGDYLDYWGQGTTLTVSS
The CD19 CAR may comprise the following VL sequence.
SEQ ID NO: 8
VL sequence from CAT19 murine monoclonal antibody
QIVLTQSPAIMSASPGEKVTMTCSASSSVSYMHWYQQKSGTSPKRWIYDTSKLASGVPDR
FSGSGSGTSYFLTINNMEAEDAATYYCQQWNINPLTFGAGTKLELKR
The CD19 CAR may comprise the following scFv sequence.
SEQ ID NO: 9
VH-VL scFv sequence from murine monoclonal antibody
QVQLQQSGPELVKPGASVKISCKASGYAFSSSWMNWVKQRPGKGLEWIGRIYPGDEDTN
YSGKFKDKATLTADKSSTTAYMQLSSLTSEDSAVYFCARSLLYGDYLDYWGQGTTLTVSSG
GGGSGGGGSGGGGSQIVLTQSPAIMSASPGEKVTMTCSASSSVSYMHWYQQKSGTSPK
RWIYDTSKLASGVPDRFSGSGSGTSYFLTINNMEAEDAATYYCQQWNINPLTFGAGTKLEL
KR
The CAR may consist of or comprise one of the following sequences.
SEQ ID NO: 10
CAT19 CAR using “Campana” architecture
MGTSLLCWMALCLLGADHADAQVQLQQSGPELVKPGASVKISCKASGYAFSSSWMNWVK
QRPGKGLEWIGRIYPGDEDTNYSGKFKDKATLTADKSSTTAYMQLSSLTSEDSAVYFCARS
LLYGDYLDYWGQGTTLTVSSGGGGSGGGGSGGGGSQIVLTQSPAIMSASPGEKVTMTCS
ASSSVSYMHWYQQKSGTSPKRWIYDTSKLASGVPDRFSGSGSGTSYFLTINNMEAEDAAT
YYCQQWNINPLTFGAGTKLELKRSDPTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEEDGCS
CRFPEEEEGGCELRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDTYDALH
MQALPPR
“Campana” architecture refers to a CAR with a CD8a spacer and transmembrane domain, 4-
1BB endodomain and TCR CD3z endodomain.
SEQ ID NO: 11
CAT19 CAR with an OX40-Zeta endodomain
MGTSLLCWMALCLLGADHADAQVQLQQSGPELVKPGASVKISCKASGYAFSSSWMNWVK
QRPGKGLEWIGRIYPGDEDTNYSGKFKDKATLTADKSSTTAYMQLSSLTSEDSAVYFCARS
LLYGDYLDYWGQGTTLTVSSGGGGSGGGGSGGGGSQIVLTQSPAIMSASPGEKVTMTCS
ASSSVSYMHWYQQKSGTSPKRWIYDTSKLASGVPDRFSGSGSGTSYFLTINNMEAEDAAT
YYCQQWNINPLTFGAGTKLELKRSDPTTTPAPRPPTPAPTIASQPLSLRPEACRPAAGGAV
HTRGLDFACDIYIWAPLAGTCGVLLLSLVITLYCRRDQRLPPDAHKPPGGGSFRTPIQEEQA
DAHSTLAKIRVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDTYDALHMQAL
PPR
SEQ ID NO: 12
CAT19 CAR with a CD28-Zeta endodomain
MGTSLLCWMALCLLGADHADAQVQLQQSGPELVKPGASVKISCKASGYAFSSSWMNWVK
QRPGKGLEWIGRIYPGDEDTNYSGKFKDKATLTADKSSTTAYMQLSSLTSEDSAVYFCARS
LLYGDYLDYWGQGTTLTVSSGGGGSGGGGSGGGGSQIVLTQSPAIMSASPGEKVTMTCS
ASSSVSYMHWYQQKSGTSPKRWIYDTSKLASGVPDRFSGSGSGTSYFLTINNMEAEDAAT
YYCQQWNINPLTFGAGTKLELKRSDPTTTPAPRPPTPAPTIASQPLSLRPEACRPAAGGAV
HTRGLDFACDIYIWAPLAGTCGVLLLSLVITLYCRSKRSRLLHSDYMNMTPRRPGPTRKHYQ
PYAPPRDFAAYRSRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTATKDTYDALH
MQALPPR
SEQ ID NO: 13
Third generation CD19 CAR
MGTSLLCWMALCLLGADHADAQVQLQQSGPELVKPGASVKISCKASGYAFSSSWMNWVK
QRPGKGLEWIGRIYPGDEDTNYSGKFKDKATLTADKSSTTAYMQLSSLTSEDSAVYFCARS
LLYGDYLDYWGQGTTLTVSSGGGGSGGGGSGGGGSQIVLTQSPAIMSASPGEKVTMTCS
ASSSVSYMHWYQQKSGTSPKRWIYDTSKLASGVPDRFSGSGSGTSYFLTINNMEAEDAAT
YYCQQWNINPLTFGAGTKLELKRSDPTTTPAPRPPTPAPTIASQPLSLRPEACRPAAGGAV
HTRGLDFACDIFWVLVVVGGVLACYSLLVTVAFIIFWVRSKRSRLLHSDYMNMTPRRPGPT
RKHYQPYAPPRDFAAYRSRDQRLPPDAHKPPGGGSFRTPIQEEQADAHSTLAKIRVKFSRS
ADAPAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
SEQ ID NO: 14
CD19 CAR with IgG1 hinge spacer
MGTSLLCWMALCLLGADHADAQVQLQQSGPELVKPGASVKISCKASGYAFSSSWMNWVK
QRPGKGLEWIGRIYPGDEDTNYSGKFKDKATLTADKSSTTAYMQLSSLTSEDSAVYFCARS
LLYGDYLDYWGQGTTLTVSSGGGGSGGGGSGGGGSQIVLTQSPAIMSASPGEKVTMTCS
ASSSVSYMHWYQQKSGTSPKRWIYDTSKLASGVPDRFSGSGSGTSYFLTINNMEAEDAAT
YYCQQWNINPLTFGAGTKLELKRSDPAEPKSPDKTHTCPPCPKDPKFWVLVVVGGVLACY
SLLVTVAFIIFWVRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR
SEQ ID NO: 15
CD19 CAR with hinge-CH2-CH3 of human IgG1 with FcR binding sites
mutated out
MGTSLLCWMALCLLGADHADAQVQLQQSGPELVKPGASVKISCKASGYAFSSSWMNWVK
QRPGKGLEWIGRIYPGDEDTNYSGKFKDKATLTADKSSTTAYMQLSSLTSEDSAVYFCARS
LLYGDYLDYWGQGTTLTVSSGGGGSGGGGSGGGGSQIVLTQSPAIMSASPGEKVTMTCS
ASSSVSYMHWYQQKSGTSPKRWIYDTSKLASGVPDRFSGSGSGTSYFLTINNMEAEDAAT
YYCQQWNINPLTFGAGTKLELKRSDPAEPKSPDKTHTCPPCPAPPVAGPSVFLFPPKPKDT
LMIARTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEA
LHNHYTQKSLSLSPGKKDPKFWVLVVVGGVLACYSLLVTVAFIIFWVRSKRSRLLHSDYMN
MTPRRPGPTRKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRRGKGHDGLY
QGLSTATKDTYDALHMQALPPR

The CAR provided herein may comprise a variant of the polypeptide of SEQ ID NO: 1-15 having at least 80, 85, 90, 95, 98 or 99% sequence identity, provided that the variant sequence retain the capacity to bind CD19 (when in conjunction with a complementary VL or VH domain, if appropriate).

The percentage identity between two polypeptide sequences may be readily determined by programs such as BLAST which is freely available at http://blast.ncbi.nlm.nih.gov.

The CD19 CAR exemplified herein (i.e., the CAT19CAR using “Campana” architecture, SEQ ID NO: 10) has properties contemplated by the disclosure to result in lower toxicity and better efficacy in treated patients. When compared with an fmc63-Campana CAR, the CATi9CAR exemplified herein effected killing of target cells expressing CD19 and proliferated in response to CD19 expressing targets, but Interferon-gamma release was less. Further, a small animal model of an aggressive B-cell lymphoma showed equal efficacy and equal engraftment between the fmc63- and CAT19-based CAR-T cells, but surprisingly, less of the CAT19 CAR T-cells were exhausted than fmc63 CAR T-cells. See, Examples 2 and 3 of US Publication No.: 2018-0044417.

The CAT19CAR provided herein may cause 25, 50, 70 or 90% lower IFNγ release in a comparative assay involving bringing CAR T cells into contact with target cells.

The CATi9CAR provided herein may result in a smaller proportion of CAR T cells becoming exhausted than fmc63 CAR T cells. T cell exhaustion may be assessed using methods known in the art, such as analysis of PD-1 expression. The CAR may cause 20, 30, 40, 50, 60 of 70% fewer CAR T cells to express PD-1 that fmc63 CAR T cells in a comparative assay involving bringing CAR T cells into contact with target cells.

Another exemplary CD19 antigen-binding domain contemplated by the disclosure is based on the CD19 antigen-binding domain CD19ALAb (described in WO2016/102965) and comprises:

a) a heavy chain variable region (VH) having CDRs
with the following sequences:
(SEQ ID NO: 16)
CDR1 - SYWMN;
(SEQ ID NO: 17)
CDR2 - QIWPGDGDTNYNGKFK
(SEQ ID NO: 18)
CDR3 - RETTTVGRYYYAMDY;
and
b) a light chain variable region (VL) having CDRs
with the following sequences:
(SEQ ID NO: 19)
CDR1 - KASQSVDYDGDSYLN;
(SEQ ID NO: 20)
CDR2 - DASNLVS
(SEQ ID NO: 21)
CDR3 - QQSTEDPWT.

It is contemplated that it is possible to introduce one or more mutations (substitutions, additions or deletions) into one or more CDRs without negatively affecting CD19-binding activity. Each CDR may, for example, have one, two or three amino acid mutations.

The CAR may comprise one of the following amino
acid sequences.
SEQ ID NO: 22
Murine CD19ALAb scFv sequence
QVQLQQSGAELVRPGSSVKISCKASGYAFSSYWMNWVKQRPGQGLEWIGQ
IWPGDGDTNYNGKFKGKATLTADESSSTAYMQLSSLASEDSAVYFCARRE
TTTVGRYYYAMDYWGQGTTVTVSSDIQLTQSPASLAVSLGQRATISCKAS
QSVDYDGDSYLNWYQQIPGQPPKLLIYDASNLVSGIPPRFSGSGSGTDFT
LNIHPVEKVDAATYHCQQSTEDPWTFGGGTKLEIK
SEQ ID NO: 23
Humanized CD19ALAb scFv sequence - Heavy 19, Kappa
16
QVQLVQSGAEVKKPGASVKLSCKASGYAFSSYWMNWVRQAPGQSLEWIGQ
IWPGDGDTNYNGKFKGRATLTADESARTAYMELSSLRSGDTAVYFCARRE
TTTVGRYYYAMDYWGKGTLVTVSSDIQLTQSPDSLAVSLGERATINCKAS
QSVDYDGDSYLNWYQQKPGQPPKLLIYDASNLVSGVPDRFSGSGSGTDFT
LTISSLQAADVAVYHCQQSTEDPWTFGQGTKVEIKR
SEQ ID NO: 24
(Humanized CD19ALAb scFv sequence - Heavy 19,
Kappa 7)
QVQLVQSGAEVKKPGASVKLSCKASGYAFSSYWMNWVRQAPGQSLEWIGQ
IWPGDGDTNYNGKFKGRATLTADESARTAYMELSSLRSGDTAVYFCARRE
TTTVGRYYYAMDYWGKGTLVTVSSDIQLTQSPDSLAVSLGERATINCKAS
QSVDYDGDSYLNWYQQKPGQPPKVLIYDASNLVSGVPDRFSGSGSGTDFT
LTISSLQAADVAVYYCQQSTEDPWTFGQGTKVEIKR
The scFv may be in a VH-VL orientation (as shown
in SEQ ID NO:s 9, 22, 23 and 24) or a VL-VH
orientation.
The CAR may comprise one of the following VH
sequences:
SEQ ID NO: 25
Murine CD19ALAb VH sequence
QVQLQQSGAELVRPGSSVKISCKASGYAFSSYWMNWVKQRPGQGLEWIGQ
IWPGDGDTNYNGKFKGKATLTADESSSTAYMQLSSLASEDSAVYFCARRE
TTTVGRYYYAMDYWGQGTTVTVSS
SEQ ID NO: 26
Humanized CD19ALAb VH sequence
QVQLVQSGAEVKKPGASVKLSCKASGYAFSSYWMNWVRQAPGQSLEWIGQ
IWPGDGDTNYNGKFKGRATLTADESARTAYMELSSLRSGDTAVYFCARRE
TTTVGRYYYAMDYWGKGTLVTVSS
The CAR may comprise one of the following VL
sequences:
SEQ ID NO: 27
Murine CD19ALAb VL sequence
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIK
SEQ ID NO: 28
(Humanized CD19ALAb VL sequence, Kappa 16)
DIQLTQSPDSLAVSLGERATINCKASQSVDYDGDSYLNWYQQKPGQPPKL
LIYDASNLVSGVPDRFSGSGSGTDFTLTISSLQAADVAVYHCQQSTEDPW
TFGQGTKVEIKR
SEQ ID NO: 29
Humanized CD19ALAb VL sequence, Kappa 7
DIQLTQSPDSLAVSLGERATINCKASQSVDYDGDSYLNWYQQKPGQPPKV
LIYDASNLVSGVPDRFSGSGSGTDFTLTISSLQAADVAVYYCQQSTEDPW
TFGQGTKVEIKR

The CAR provided herein may comprise a variant of the sequence shown as any of SEQ ID NO: 16-29 having at least 80, 85, 90, 95, 98 or 99% sequence identity, provided that the variant sequence retain the capacity to bind CD19 (when in conjunction with a complementary VL or VH domain, if appropriate).

The percentage identity between two polypeptide sequences may be readily determined by programs such as BLAST which is freely available at blast.ncbi.nlm.nih.gov.

Binding domains specific for CD22 target antigen

The human CD22 antigen is a molecule belonging to the SIGLEC family of lectins. It is found on the surface of mature B cells and on some immature B cells. Generally speaking, CD22 is a regulatory molecule that prevents the overactivation of the immune system and the development of autoimmune diseases.

CD22 is a sugar-binding transmembrane protein, which specifically binds sialic acid with an immunoglobulin (Ig) domain located at its N-terminus. The presence of Ig domains makes CD22 a member of the immunoglobulin superfamily. CD22 functions as an inhibitory receptor for B cell receptor (BCR) signaling.

CD22 is a molecule of the IgSF which may exist in two isoforms, one with seven domains and an intra-cytoplasmic tail comprising of three ITIMs (immune receptor tyrosine-based inhibitory motifs) and an ITAM; and a splicing variant which instead comprises of five extracellular domains and an intra-cytoplasmic tail carrying one ITIM. CD22 is thought to be an inhibitory receptor involved in the control of B-cell responses to antigen. Like CD19, CD22 is widely considered to be a pan-B antigen, although expression on some non-lymphoid tissue has been described. CD22-targeted therapeutic monoclonal antibodies and immunoconjugates have entered clinical testing.

The antigen-binding domain of the CAR which binds to CD22 may be any domain which is capable of binding CD22. For example, the antigen-binding domain may comprise a CD22 binder as described in Table 3.

TABLE 3

| Antigen-binding | |
|---|---|
| domain | References |
| M5/44 or | John et al., J. Immunol. Baltim. Md 1950, 170: 3534- |
| humanized | 3543 (2003); and DiJoseph et al., Cancer Immunol. |
| M5/44 | Immunother. CII, 54: 11-24 (2005) |
| M6/13 | DiJoseph et al., supra |
| HD39 | Dorken et al., J. Immunol. Baltim. Md 1950, 136: |
| | 4470-4479 (1986) |
| HD239 | Dorken et al., supra |
| HD6 | Pezzutto et al., J. Immunol. Baltim. Md 1950, 138: |
| | 98-103 (1987) |
| RFB-4, or | Campana et al., J. Immunol. Baltim. Md 1950, 134: |
| humanized | 1524-1530 (1985); Krauss et al., Protein Eng., 16: |
| RFB-4, or | 753-759 (2003), Kreitman et al., J. Clin. Oncol. Off. J. |
| affinity | Am. Soc. Clin. Oncol., 30: 1822-1828 (2012) |
| matured | |
| To15 | Mason et al., Blood, 69: 836-840 (1987) |
| 4KB128 | Mason et al., supra |
| S-HCL1 | Schwarting et al., Blood, 65: 974-983 (1985) |
| mLL2 (EPB-2), | Shih et al., Int. J. Cancer J. Int. Cancer, 56: 538-545 |
| or humanized | (1994), Leonard et al., J. Clin. Oncol. Off. J. Am. Soc. |
| mLL2 - hLL2 | Clin. Oncol., 21: 3051-3059 (2003) |
| M971 | Xiao et al., mAbs, 1: 297-303 (2009) |
| BC-8 | Engel et al., J. Exp. Med., 181: 1581-1586 (1995) |
| HB22-12 | Engel et al., supra |

Other anti-CD22 antibody-binding domains are known, such as the mouse anti-human CD22 antibodies 1QD9-3, 3K4-13, 7G6-6, 64-6, 4D9-12, 54-9, 1001-D9, 15G7-2, 21B12-8, 2C4-4 and 3E10-7; and the humanized anti-human CD22 antibodies LT22 and Inotuzumab (G5_44). Table 4 presents VH, VL and CDR sequences (in bold and underlined) and the position of the target epitope on CD22 for each antibody.

TABLE 4

| | | | Position of |
|---|---|---|---|
| | | | epitope on |
| Antibody | VH | VL | CD22 |
| 1D9-3 | EVQLVESGGGLVQPKGSLK | DIVMTQSQKFMSTSVGDR | Domain 1 |
| | LSCAASGFTFNTYAMHWVR | VSITCKASQNVRTAVAWY | and 2 |
| | QAPGKGLEWVARIRSKSSN | QQKPGQSPKALIYLASNR | |
| | YATYYADSVKDRFTISRDD | HTGVPDRFTGSGSGTDFT | |
| | SQSMLYLQMNNLKTEDTAM | LTISNVQSEDLADYFCLQH | |
| | YYCVVDYLYAMDYWGQGT | WNYPFTFGSGTKLEIK | |
| | SVTVSS | (SEQ ID NO: 31) | |
| | (SEQ ID NO: 30) | | |
| 3B4-13 | QVQLQQSGAELVRPGASVT | QAVVTQESALTTSPGETV | Domain 1 |
| | LSCKASGYTFTDYEMHWVK | TLTCRSSAGAVTTSNYAN | and 2 |
| | QTPVHGLEWIGAIDPETGA | WVQEKPDHLFTGLIGGTN | |
| | TAYNQKFKGKAILTADKSSS | NRAPGVPARFSGSLIGDK | |
| | TAYMDLRSLTSEDSAVYYC | AALTITGAQTEDEAIYFCAL | |
| | TRYDYGSSPWFAYWGQGT | WNSNHWVFGGGTKLTVL | |
| | LVTVSA | (SEQ ID NO: 33) | |
| | (SEQ ID NO: 32) | | |
| 7G6-6 | QVQLQQPGAELVMPGASV | DIVMSQSPSSLAVSVGEK | Domain 1 |
| | KLSCKASGYTFTSYWMHW | VTMSCKSSQSLLYSSNQK | and 2 |
| | VKQRPGQGLEWIGEIDPSD | NYLAWYQQKPGQSPKLLI | |
| | SYTNYNQKFKGKATLTVDK | YWASTRESGVPDRFTGS | |
| | SSSTAYMQLSSLTSEDSAV | GSGTDFTLTISSVKAEDLA | |
| | YYCARGYYGSSSFDYWGQ | VYYCQQYYSYTFGGGTKL | |
| | GTTLTVSS | EIK | |
| | (SEQ ID NO: 34) | (SEQ ID NO: 35) | |
| 6C4-6 | QVQLKESGPGLVAPSQSLSI | DIQMTQSPASLSASVGET | Domain 3 |
| | TCTVSGFSLTSYGVHWVRQ | VTITCRASENIYSYLAWYQ | |
| | PPGKGLEWLVVIWSDGSTT | QKQGKSPQLLVYNAKTLA | |
| | YNSALKSRLSISKDNSKSQ | EGVPSRFSGSGSGTQFSL | |
| | VFLKMNSLQTDDTAMYYCA | KINSLQPEDFGSYYCQHH | |
| | RHADDYGFAWFAYWGQG | YGTPPTFGGGTKLEIK | |
| | TLVTVSA | (SEQ ID NO: 37) | |
| | (SEQ ID NO: 36) | | |
| 4D9-12 | EFQLQQSGPELVKPGASVK | DIQMTQSPSSLSASLGER | Domain 4 |
| | ISCKASGYSFTDYNMNWVK | VSLTCRASQEISGYLSWL | |
| | QSNGKSLEWIGVINPNYGT | QQKPDGTIKRLIYAASTLD | |
| | TSYNQKFKGKATLTVDQSS | SGVPKRFSGSRSGSDYSL | |
| | STAYMQLNSLTSEDSAVYY | TISSLESEDFADYYCLQYA | |
| | CARSSTTVVDWYFDVWGT | SYPFTFGSGTKLEIK | |
| | GTTVTVSS | (SEQ ID NO: 39) | |
| | (SEQ ID NO: 38) | | |
| 5H4-9 | QVQVQQPGAELVRPGTSV | DVVMTQTPLSLPVSLGDQ | Domain 4 |
| | KLSCKASGYTFTRYWMYW | ASISCRSSQSLVHSNGNT | |
| | VKQRPGQGLEWIGVIDPSD | YLHWYLQKPGQSPKLLIY | |
| | NFTYYNQKFKGKATLTVDT | KVSNRFSGVPDRFSGSGS | |
| | SSSTAYMQLSSLTSEDSAV | GTDFTLKISRVEAEDLGVY | |
| | YYCARGYGSSYVGYWGQG | FCSQSTHVPPWTFGGGTK | |
| | TTLTVSS | LEIK | |
| | (SEQ ID NO: 40) | (SEQ ID NO: 41) | |
| 10C1-D9 | QVTLKESGPGILQSSQTLSL | DIQMTQTTSSLSASLGDRV | Domain 4 |
| | TCSFSGFSLSTSDMGVSWI | TISCRASQDISNYLNWYQ | |
| | RQPSGKGLEWLAHIYWDD | QKPDGTVKLLIYYTSRLHS | |
| | DKRYNPSLKSRLTISKDASR | GVPSRFSGSGSGTDYSLTI | |
| | NQVFLKIATVDTADTATYYC | SNLEQEDIATYFCQQGNT | |
| | ARSPWIYYGHYWCFDVWG | LPFTFGSGTKLEIK | |
| | TGTTVTVSS | (SEQ ID NO: 43) | |
| | (SEQ ID NO: 42) | | |
| 15G7-2 | QVQLQQSGAELVKPGASVK | QIVLTQSPAIMSASPGEKV | Domain 4 |
| | LSCKASGYTFTEYTIHWVK | TMTCSASSSVSYMYWYQ | |
| | QRSGQGLEWIGWFYPGSG | QKPGSSPRLLIYDTSNLAS | |
| | SIKYNEKFKDKATLTADKSS | GVPVRFSGSGSGTSYSLTI | |
| | STVYMELSRLTSEDSAVYF | SRMEAEDAATYYCQQWS | |
| | CARHGDGYYLPPYYFDYW | SYPLTFGAGTKLELK | |
| | GQGTTLTVSS | (SEQ ID NO: 45) | |
| | (SEQ ID NO: 44) | | |
| 2B12-8 | QVQLQQSGAELARPGASVK | DIVLTQSPATLSVTPGDSV | Domain 4 |
| | LSCKASGYIFTSYGISWVKQ | SLSCRASQSISTNLHWYQ | |
| | RTGQGLEWIGEIYPRSGNT | QKSHASPRLLIKYASQSVS | |
| | YYNEKFKGKATLTADKSSS | GIPSRFSGSGSGTDFTLSI | |
| | TAYMELRSLTSEDSAVYFC | NSVETEDFGIFFCQQSYS | |
| | ARPIYYGSREGFDYWGQGT | WPYTFGGGTKLEIK | |
| | TLTVSS | (SEQ ID NO: 47) | |
| | (SEQ ID NO: 46) | | |
| 2C4-4 | QVQLQQPGAELVMPGASV | DVLMTQTPLSLPVSLGDQ | Domain 5-7 |
| | KLSCKASGYTFTSYWMHW | ASISCRSSQSIVHSNGNTY | |
| | VKQRPGQGLEWIGEIDPSD | LEWYLQKPGQSPKLLIYKV | |
| | SYTNYNQKFKGKSTLTVDK | SNRFSGVPDRFSGSESGT | |
| | SSSTAYIQLSSLTSEDSAVY | DFTLKISRVEAEDLGVYYC | |
| | YCARWASYRGYAMDYWG | FQGSHVPWTFGGGTKLEI | |
| | QGTSVTVSS | K | |
| | (SEQ ID NO: 48) | (SEQ ID NO: 49) | |
| 3E10-7 | EFQLQQSGPELVKPGASVK | DIQMTQSPSSLSASLGER | Domain 5-7 |
| | ISCKASGYSFTDYNMNWVK | VSLTCRASQEISGYLSWL | |
| | QSNGKSLEWIGVINPNYGT | QQKPDGTIKRLIYAASTLD | |
| | TSYNQRFKGKATLTVDQSS | SGVPKRFSGSRSGSDYSL | |
| | STAYMQLNSLTSEDSAVYY | TISSLESEDFADYYCLQYA | |
| | CARSGLRYWYFDVWGTGT | SYPFTFGSGTKLEIK | |
| | TVTVSS | (SEQ ID NO: 51) | |
| | (SEQ ID NO: 50) | | |
| LT22 | EVQLVESGAEVKKPGSSVK | DIVMTQSPATLSVSPGERA | Domain 5 |
| | VSCKASGYTFTNYWINWVR | TLSCRSSQSLVHSNGNTY | |
| | QAPGQGLEWMGNIYPSDS | LHWYQQKPGQAPRLLIYK | |
| | FTNYNQKFKDRVTITADKST | VSNRFSGVPARFSGSGSG | |
| | STVYLELRNLRSDDTAVYY | AEFTLTISSLQSEDFAVYY | |
| | CTRDTQERSWYFDVWGQG | CSQSTHVPWTFGQGTRLE | |
| | TLVTVSS | IKR | |
| | (SEQ ID NO: 52) | (SEQ ID NO: 53) | |
| Inotuzumab | EVQLVQSGAEVKKPGASVK | DVQVTQSPSSLSASVGDR | Domain 7 |
| G5_44 | VSCKASGYRFTNYWIHWVR | VTITCRSSQSLANSYGNTF | |
| | QAPGQGLEWIGGINPGNNY | LSWYLHKPGKAPQLLIYGI | |
| | ATYRRKFQGRVTMTADTST | SNRFSGVPDRFSGSGSGT | |
| | STVYMELSSLRSEDTAVYY | DFTLTISSLQPEDFATYYC | |
| | CTREGYGNYGAWFAYWG | LQGTHQPYTFGQGTKVEI | |
| | QGTLVTVSS | KR | |
| | (SEQ ID NO: 54) | (SEQ ID NO: 55) | |
| 1G3-4 | QVTLKESGPGILQPSQTLSL | DIQMTQSPASLSASLGETV | Domain 4 |
| | TCTFSGFSLSTSGMGVGWI | SIECLASGGISNDLAWYQ | |
| | RQPSGKGLEWLTNIWWDD | QKSGKSPQLLIYAASRLQ | |
| | DKNYNPSLKNRLTISKDTSI | DGVPSRFSGSGSGTRYSL | |
| | NQAFLKITNVDTADTATYYC | KISGMQSEDEADYFCQQS | |
| | ARIAHYFDGYYYVMDVWG | YKYPYTFGGGTKLELK | |
| | QGTSVTVSS (SEQ ID NO: | (SEQ ID NO: 57) | |
| | 56) | | |

Examples of CD22 CARs are described by Haso et al., Blood, 121(7): 1165-1174 (2013). Specifically, CD22 CARs with antigen-binding domains derived from m971, HA22 and BL22 scFvs are described.

CD22 has seven extracellular IgG-like domains, which are commonly identified as Ig domain 7 to Ig domain 1, with Ig domain 1 being most proximal to the B cell membrane and Ig domain 7 being the most distal from the Ig cell membrane.

The positions of the Ig domains in terms of the amino acid sequence of CD22 (UniProt Accession No. P20273, entry version 224, http://www.uniprot.org/uniprot/P20273) are summarized in the following Table 5:

TABLE 5

| Ig | | | | | | | |
|---|---|---|---|---|---|---|---|
| domain | 7 | 6 | 5 | 4 | 3 | 2 | 1 |
| CD22 | V-type 1 | C2-type 2 | C2-type 3 | C2-type 4 | C2-type 5 | C2-type 6 | C2-type 7 |
| domain | | | | | | | |
| Amino | 20-138 | 143-235 | 242-326 | 331-416 | 419-500 | 505-582 | 593-676 |
| acids | | | | | | | |

The antigen-binding domain of the second CAR may bind to a membrane-distal epitope on CD22, for example, Ig domain 7. The antigen-binding domain of the second CAR may bind to an epitope on Ig domain 7, 6, 5 or 4 of CD22, for example, on Ig domain 5 of CD22. The antigen-binding domain of the second CAR may bind to an epitope located between amino acids 20-416 of CD22, for example, between amino acids 242-326 of CD22.

The antigen-binding domain of the second CAR may bind to a membrane-proximal epitope on CD22. The antigen-binding domain of the second CAR may bind to an epitope on Ig domain 3, 2 or 1 of CD22. The antigen-binding domain of the second CAR may bind to an epitope located between amino acids 419-676 of CD22, such as between 505-676 of CD22.

A CD22-binding domain exemplified herein comprises variable regions with CDRs from an antibody referred to as 9A8-1 (described in WO2019/220109):

a) a heavy chain variable region (VH) having 9A8-1
CDRs with the following sequences:
(SEQ ID NO: 58)
CDR1 - NFAMA;
(SEQ ID NO: 59)
CDR2 - SISTGGGNTYYRDSVKG
(SEQ ID NO: 60)
CDR3 - QRNYYDGSYDYEGYTMDA;
and
b) a light chain variable region (VL) having 9A8-1
CDRs with the following sequences:
(SEQ ID NO: 61)
CDR1 - RSSQDIGNYLT;
(SEQ ID NO: 62)
CDR2 - GAIKLED
(SEQ ID NO: 63)
CDR3 - LQSIQYP.
The CD22 CAR may comprise the following VH
sequence.
SEQ ID NO: 64
VH sequence from 9A8-1 antibody
EVQLVESGGGLVQPGRSLKLSCAASGFTFSNFAMAWVRQPPTKGLEWVAS
ISTGGGNTYYRDSVKGRFTISRDDAKNTQYLQMDSLRSEDTATYYCARQR
NYYDGSYDYEGYTMDAWGQGTSVTVSS
The CD22 CAR may comprise the following VL
sequence.
SEQ ID NO: 65
VL sequence from 9A8-1 antibody
DIQMTQSPSSLSASLGDRVTITCRSSQDIGNYLTWFQQKVGRSPRRMIYG
AIKLEDGVPSRFSGSRSGSDYSLTISSLESEDVADYQCLQSIQYPFTFGS
GTKLEIK
The CD22 CAR may comprise the following scFv
sequence.
SEQ ID NO: 66
VL-VH scFv sequence based on 9A8-1 VH and VL
sequences
DIQMTQSPSSLSASLGDRVTITCRSSQDIGNYLTWFQQKVGRSPRRMIYG
AIKLEDGVPSRFSGSRSGSDYSLTISSLESEDVADYQCLQSIQYPFTFGS
GTKLEIKRSGGGGSGGGGSGGGGSEVQLVESGGGLVQPGRSLKLSCAASG
FTFSNFAMAWVRQPPTKGLEWVASISTGGGNTYYRDSVKGRFTISRDDAK
NTQYLQMDSLRSEDTATYYCARQRNYYDGSYDYEGYTMDAWGQGTSVTVS

The antigen-binding domain of the 9A8-1 antibody exhibits particularly good efficacy in a CAR. For example, 9A8-1 in a FabCAR format showed improved target cell killing and cytokine release that an equivalent CAR with an alternative CD22 binder, 3B4 (WO2019/220109).

An antigen-binding domain of a CAR which binds to CD22 may comprise the VH and/or VL sequence from any of the CD22 antibodies listed above, or a variant thereof which has at least 70, 80, 90 or 90% sequence identity, which variant retains the capacity to bind CD22.

The antigen-binding domain of a CD22 CAR may bind CD22 with a KD in the range 30-50 nM, for example 30-40 nM. The KD may be about 32 nM.

### OR Gates

The CAR may be used in a combination with one or more other activating or inhibitory chimeric antigen receptors. For example, they may be used in combination with one or more other CARs in a “logic-gate”, a CAR combination which, when expressed by a cell, such as a T cell, are capable of detecting a particular pattern of expression of at least two target antigens. If the at least two target antigens are arbitrarily denoted as antigen A and antigen B, the three possible options are as follows:
“OR GATE”—T cell triggers when either antigen A or antigen B is present on the target cell
“AND GATE”—T cell triggers only when both antigens A and B are present on the target cell
“AND NOT GATE”—T cell triggers if antigen A is present alone on the target cell, but not if both antigens A and B are present on the target cell

Engineered T cells expressing these CAR combinations can be tailored to be exquisitely specific for cancer cells, based on their particular expression (or lack of expression) of two or more markers.

Such “Logic Gates” are described, for example, in WO2015/075469, WO2015/075470 and WO2015/075470.

An “OR Gate” comprises two or more activatory CARs each directed to a distinct target antigen expressed by a target cell. The advantage of an OR gate is that the effective targetable antigen is increased on the target cell, as it is effectively antigen A + antigen B. This is especially important for antigens expressed at variable or low density on the target cell, as the level of a single antigen may be below the threshold needed for effective targeting by a CAR-T cell. Also, it avoids the phenomenon of antigen escape. For example, some lymphomas and leukemias become CD19 negative after CD19 targeting: using an OR gate which targets CD19 in combination with another antigen provides a “back-up” antigen, should this occur.

The OR gate may comprise a CAR against a second antigen expressed in B cells, such as CD22.

Thus, the antigen-binding domains of the first and second CARs bind to different antigens and both CARs may comprise an activating endodomain. The two CARs may comprise spacer domains which may be the same, or sufficiently different to prevent cross-pairing of the two different receptors. As contemplated herein a cell can hence be engineered to activate upon recognition of either or both CD19 and CD22. This is useful in the field of oncology as indicated by the Goldie-Coldman hypothesis: sole targeting of a single antigen may result in tumor escape by modulation of said antigen due to the high mutation rate inherent in most cancers. By simultaneously targeting two antigens, the probably of such escape is exponentially reduced.

It is important that the two CARs do not heterodimerize.

The first and second CAR of the T cell may be produced as a polypeptide comprising both CARs, together with a cleavage site.

### Signal Peptides

The CARs of the cell may comprise a signal peptide so that when the CAR is expressed inside a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The signal peptide may comprise the amino acid sequence of any of SEQ ID NO: 68-70 or a variant thereof having 5, 4, 3, 2 or 1 amino acid mutations (insertions, substitutions or additions) provided that the signal peptide still functions to cause cell surface expression of the CAR.

The signal peptide of SEQ ID NO: 67 is compact and highly efficient. It is predicted to give about 95% cleavage after the terminal glycine, giving efficient removal by signal peptidase.

SEQ ID NO: 67
MGTSLLCWMALCLLGADHADA

The signal peptide of SEQ ID NO: 68 follows.

METDTLLLWVLLLLVPGSTG

The signal peptide of SEQ ID NO: 2 is derived from IgG1.

SEQ ID NO: 2: MSLPVTALLLPLALLLHAARP

The signal peptide of SEQ ID NO: 3 is derived from CD8.

SEQ ID NO: 3: MAVPTQVLGLLLLWLTDARC

The signal peptide for the first CAR may have a different sequence from the signal peptide of the second CAR.

### Spacers

CARs comprise a spacer to connect the antigen-binding domain with the transmembrane domain and spatially separate the antigen-binding domain from the endodomain. A flexible spacer allows the antigen-binding domain to orient in different directions to facilitate binding.

The spacer may, for example, comprise an IgG1 Fc region, an IgG1 hinge or a CD8 stalk, or a combination thereof. The spacer may alternatively comprise an alternative sequence which has similar length and/or domain spacing properties as an IgG1 Fc region, an IgG1 hinge or a CD8 stalk.

In the cells provided herein, the first and second CARs may comprise different spacer molecules. For example, the spacer may, for example, comprise an IgG1 Fc region, an IgG1 hinge or a human CD8 stalk or the mouse CD8 stalk. The spacer may alternatively comprise an alternative linker which has similar length and/or domain spacing properties as an IgG1 Fc region, an IgG1 hinge or a CD8 stalk. A human IgG1 spacer may be altered to remove Fc binding motifs.

The spacer for the CD19 CAR may comprise a CD8 stalk spacer, or a spacer having a length equivalent to a CD8 stalk spacer. The spacer for the CD19 CAR may have at least 30 amino acids or at least 40 amino acids. It may have between 35-55 amino acids, for example between 40-50 amino acids. It may have about 46 amino acids.

The spacer for the CD22 CAR may comprise an IgG1 hinge spacer, or a spacer having a length equivalent to an IgG1 hinge spacer. The spacer for the CD22 CAR may have fewer than 30 amino acids or fewer than 25 amino acids. It may have between 15-25 amino acids, for example between 18-22 amino acids. It may have about 20 amino acids.

Examples of amino acid sequences for these spacers are given below:

SEQ ID NO: 71
(hinge-CH2CH3 of human IgG1)
AEPKSPDKTHTCPPCPAPPVAGPSVFLFPPKPKDTLMIARTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGKKD
SEQ ID NO: 72
(human CD8 stalk):
TTTPAPRPPTPAPTIASQPLSLRPEACRPAAGGAVHTRGLDFACD
SEQ ID NO: 73
(human IgG1 hinge):
AEPKSPDKTHTCPPCPKDPK
SEQ ID NO: 74
(IgG1 Hinge-Fc)
AEPKSPDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPGKKDPK
SEQ ID NO: 75
(IgG1 Hinge - Fc modified to remove Fc receptor
recognition motifs)
AEPKSPDKTHTCPPCPAPPVA*GPSVFLFPPKPKDTLMIARTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPGKKDPK
Modified residues are underlined; * denotes a
deletion.
SEQ ID NO: 76
(CD2 ectodomain)
KEITNALETWGALGQDINLDIPSFQMSDDIDDIKWEKTSDKKKIAQFRKE
KETFKEKDTYKLFKNGTLKIKHLKTDDQDIYKVSIYDTKGKNVLEKIFDL
KIQERVSKPKISWTCINTTLTCEVMNGTDPELNLYQDGKHLKLSQRVITH
KWTTSLSAKFKCTAGNKVSKESSVEPVSCPEKGLD
SEQ ID NO: 77
(CD34 ectodomain)
SLDNNGTATPELPTQGTFSNVSTNVSYQETTTPSTLGSTSLHPVSQHGNE
ATTNITETTVKFTSTSVITSVYGNTNSSVQSQTSVISTVFTTPANVSTPE
TTLKPSLSPGNVSDLSTTSTSLATSPTKPYTSSSPILSDIKAEIKCSGIR
EVKLTQGICLEQNKTSSCAEFKKDRGEGLARVLCGEEQADADAGAQVCSL
LLAQSEVRPQCLLLVLANRTEISSKLQLMKKHQSDLKKLGILDFTEQDVA
SHQSYSQKT

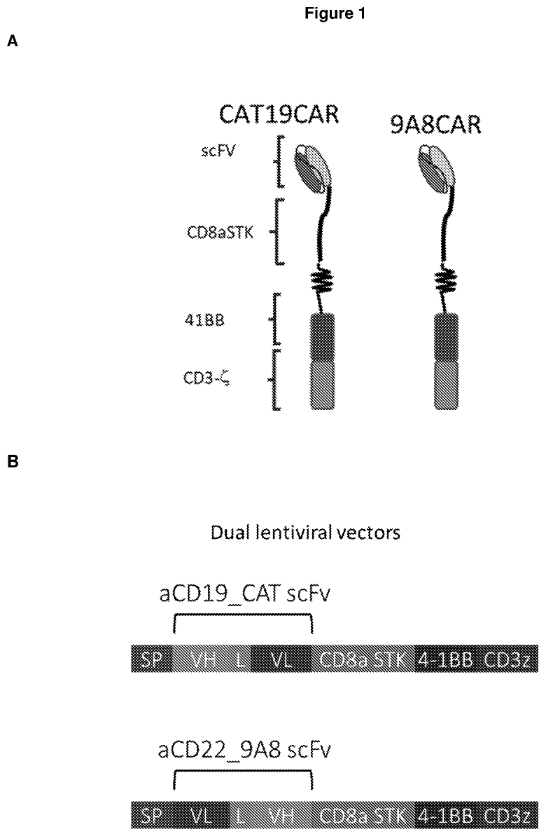

Since CARs are typically homodimers (see FIG. 1A), cross-pairing may result in a heterodimeric chimeric antigen receptor. This is undesirable for various reasons, for example: (1) the epitope may not be at the same “level” on the target cell so that a cross-paired CAR may only be able to bind to one antigen; (2) the VH and VL from the two different scFv could swap over and either fail to recognize target or worse recognize an unexpected and unpredicted antigen. The spacer of the first CAR may be sufficiently different from the spacer of the second CAR in order to avoid cross-pairing. The amino acid sequence of the first spacer may share less that 50%, 40%, 30% or 20% identity at the amino acid level with the second spacer.

### Transmembrane Domains

The transmembrane domain is the domain of the CAR that spans the membrane.

A transmembrane domain may be any protein structure which is thermodynamically stable in a membrane. This is typically an alpha helix comprising of several hydrophobic residues. The transmembrane domain of any transmembrane protein can be used to supply the transmembrane portion provided herein. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e, a polypeptide predicted to form a hydrophobic alpha helix of sufficient length to span the membrane, an artificially designed transmembrane domain may also be used (U.S. Pat. No. 7,052,906 B1 describes synthetic transmembrane components).

The transmembrane domain may be derived from CD28, which gives good receptor stability.

The transmembrane domain may be derived from human Tyrp-1. The tyrp-1 transmembrane domain sequence is shown as SEQ ID NO: 78.

SEQ ID NO: 78
IIAIAVVGALLLVALIFGTASYLI

The transmembrane domain may be derived from CD8A. The CD8A transmembrane domain sequence is shown as SEQ ID NO: 79.

SEQ ID NO: 79
IYIWAPLAGTCGVLLLSLVITLYC

### Endodomains

As noted above, the endodomain is the signal-transmission portion of the CAR. After antigen recognition, receptors cluster, native CD45 and CD148 are excluded from the synapse and a signal is transmitted to the cell. The most commonly used endodomain component is that of CD3-zeta which contains three ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The cells provided herein comprise two CARs, each with an endodomain.

The endodomain of the first CAR and the endodomain of the second CAR may comprise: (i) an ITAM-containing endodomain, such as the endodomain from CD3 zeta; and/or (ii) a co-stimulatory domain, such as the endodomain from CD28; and/or (iii) a domain which transmits a survival signal, for example a TNF receptor family endodomain such as OX-40 or 4-1BB.

Thus, the endodomain of the CAR of the present invention may comprise combinations of one or more of the CD3-Zeta endodomain, the 41BB endodomain, the OX40 endodomain or the CD28 endodomain.

The intracellular T-cell signalling domain (endodomain) of the CAR of the present invention may comprise the sequence shown as any of SEQ ID NO: 80-87 or a variant thereof having at least 80% sequence identity.

SEQ ID NO: 80
(CD3 zeta endodomain)
RSRVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTATK
DTYDALHMQALPPR
SEQ ID NO: 81
(41BB endodomain)
KRGRKKLLYIFKQPFMRPVQTTQEEDGCSCRFPEEEEGGCEL
SEQ ID NO: 82
(OX40 endodomain)
RRDQRLPPDAHKPPGGGSFRTPIQEEQADAHSTLAKI
SEQ ID NO: 83
(CD28 endodomain)
KRSRLLHSDYMNMTPRRPGPTRKHYQPYAPPRDFAAY
Examples of combinations of such endodomains
include 41BB-Zeta, OX40-Zeta, CD28-Zeta and CD28-
OX40-Zeta.
SEQ ID NO: 84
(41BB-Zeta endodomain fusion)
KRGRKKLLYIFKQPFMRPVQTTQEEDGCSCRFPEEEEGGCELRVKFSRSA
DAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQA
LPPR
SEQ ID NO: 85
(OX40-Zeta endodomain fusion)
RRDQRLPPDAHKPPGGGSFRTPIQEEQADAHSTLAKIRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
SEQ ID NO: 86
(CD28Zeta endodomain fusion)
KRSRLLHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R
SEQ ID NO: 87
(CD28OXZeta)
KRSRLLHSDYMNMTPRRPGPTRKHYQPYAPPRDFAAYRSRDQRLPPDAHK
PPGGGSFRTPIQEEQADAHSTLAKIRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR

A variant sequence may have at least 80%, 85%, 90%, 95%, 98% or 99% sequence identity to any of SEQ ID NO: 80-87 provided that the sequence provides an effective transmembrane domain/intracellular T cell signaling domain.

### Nucleic Acids

One or more nucleic acid(s) provided herein encode a CD19 CAR and a CD22 CAR of the disclosure. As used herein, the terms “polynucleotide”, “nucleotide”, and “nucleic acid” are intended to be synonymous with each other.

The nucleic acid may be, for example, an RNA, a DNA or a cDNA. Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

Alternative codons may be used in regions of sequence encoding the same or similar amino acid sequences, in order to avoid homologous recombination when the both CARs are encoded by the same vector.

Due to the degeneracy of the genetic code, it is possible to use alternative codons which encode the same amino acid sequence. For example, the codons “ccg” and “cca” both encode the amino acid proline, so using “ccg” may be exchanged for “cca” without affecting the amino acid in this position in the sequence of the translated protein.

The alternative RNA codons which may be used to encode each amino acid are summarized in Table 6.

TABLE 6

| | U | | | C | | | A | | | G | | |
|---|---|---|---|---|---|---|---|---|---|---|---|---|
| U | UUU | | Phe (F) | UCU | | | UAU | | Tyr (Y) | UGU | | Cys (C) |
| | | {close oversize brace} | | | | | | {close oversize brace} | | | {close oversize brace} | |
| | UUC | | | UCC | | | UAC | | | UGC | | |
| | UUA | | Leu (L) | UCA | {close oversize brace} | Ser (S) | UAA | | Ocher | UGA | | Opal |
| | | {close oversize brace} | | | | | | | | | {close oversize brace} | |
| | UUG | | | UCG | | | UAG | | Amber | UGG | | Trp(W) |
| C | CUU | | | CCU | | | CAU | | His (H) | CGU | | |
| | | | | | | | | {close oversize brace} | | | | |
| | CUC | | | CCC | | | CAC | | | CGC | | |
| | CUA | {close oversize brace} | Leu (L) | CCA | {close oversize brace} | Pro (P) | CAA | | Gln (Q) | CGA | {close oversize brace} | Arg (R) |
| | | | | | | | | {close oversize brace} | | | | |
| | CUG | | | CCG | | | CAG | | | CCG | | |
| A | AUU | | | ACU | | | AAU | | Asn (N) | AGU | | Ser (S) |
| | | | | | | | | {close oversize brace} | | | {close oversize brace} | |
| | AUC | {close oversize brace} | Ile (I) | ACC | | | AAC | | | AGC | | |
| | AUA | | | ACG | {close oversize brace} | Thr (T) | AAA | | Lys (K) | AGA | | Arg (R) |
| | | | | | | | | {close oversize brace} | | | {close oversize brace} | |
| | AUG | | Met(M) | ACG | | | AAG | | | AGG | | |
| G | GUU | | | GCU | | | GAU | | Asp (D) | GGU | | |
| | | | | | | | | {close oversize brace} | | | | |
| | GUC | | | GCC | | | GAU | | | GGC | | |
| | GUA | {close oversize brace} | Val (V) | GCA | {close oversize brace} | Ala (A) | GAA | | Glu (E) | GGA | {close oversize brace} | Gly (G) |
| | | | | | | | | {close oversize brace} | | | | |
| | GUG | | | GCG | | | GAG | | | GGG | | |

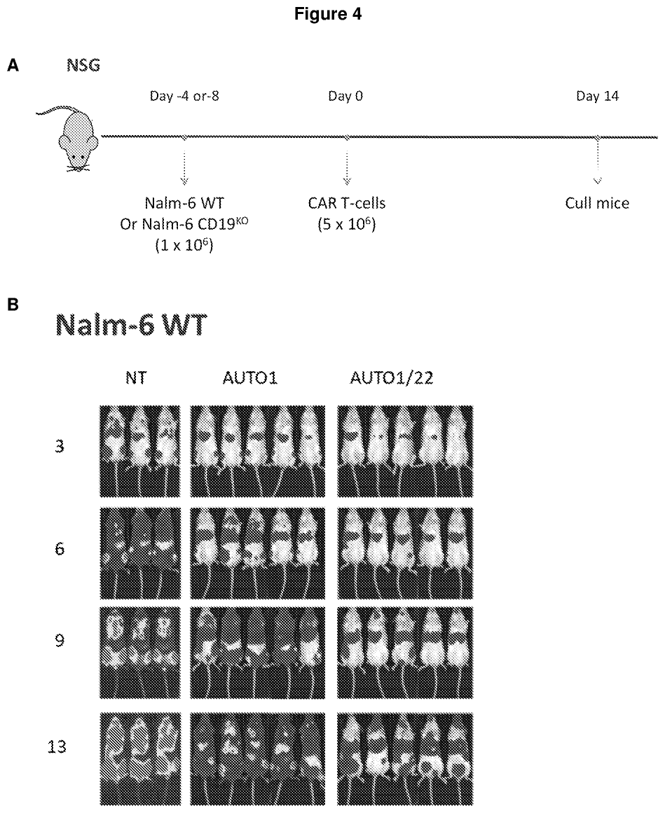

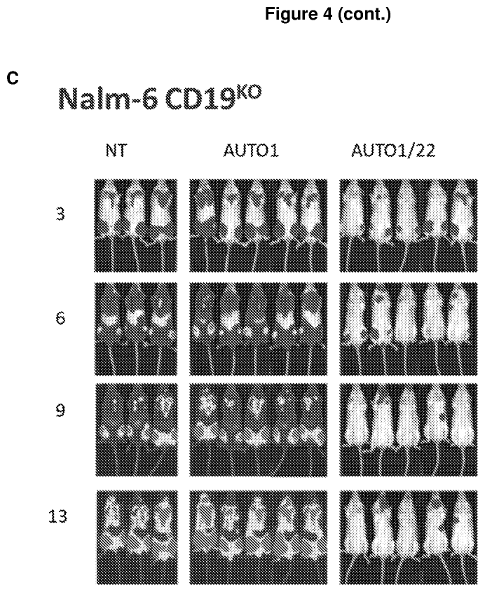

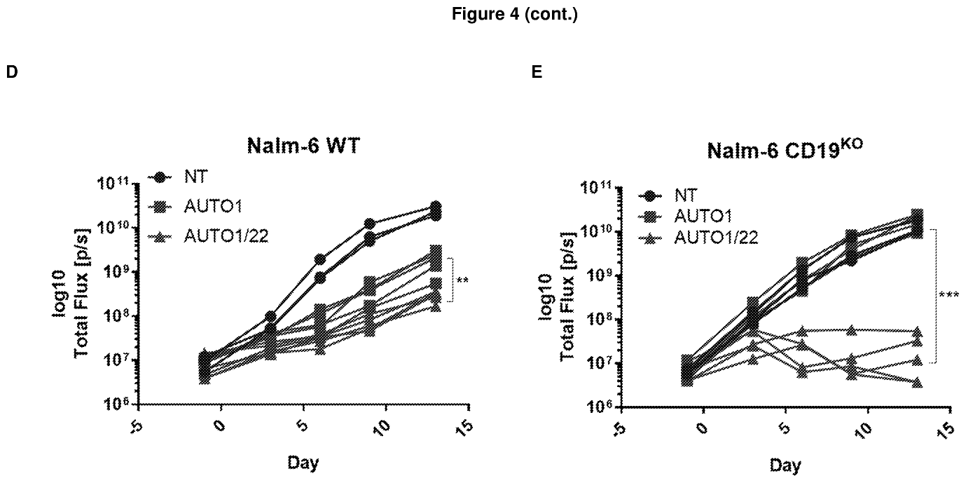

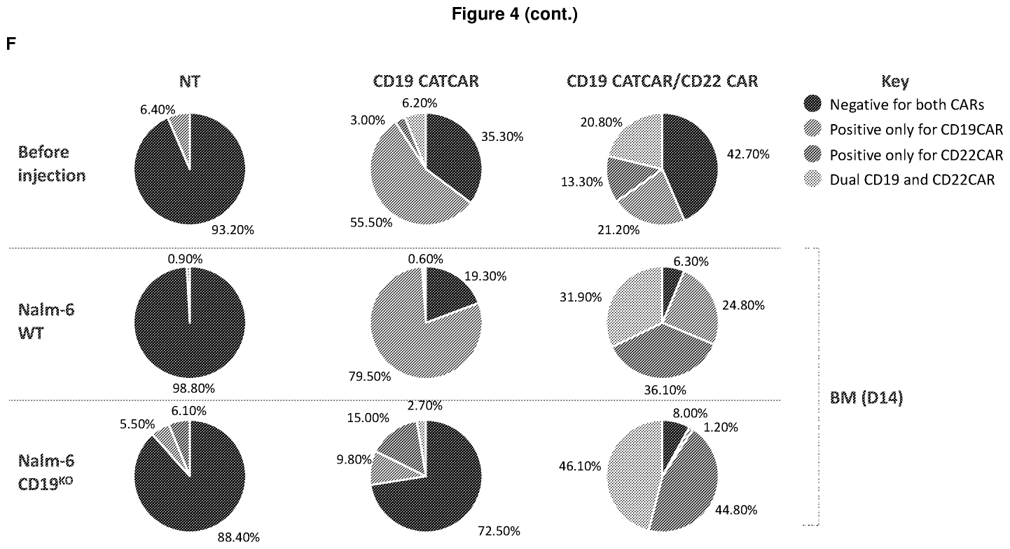

Alternative codons may be used in the portions of nucleic acid which encode the spacer of the first CAR and the spacer of the second CAR, especially if the same or similar spacers are used in the first and second CARs. FIG. 4 shows two sequences encoding the spacer HCH2CH3—hinge, in one of which alternative codons have been used.

Alternative codons may be used in the portions of nucleic acid which encode the transmembrane domain of the first CAR and the transmembrane of the second CAR, especially if the same or similar transmembrane domains are used in the first and second CARs.

Alternative codons may be used in one or more nucleic acids which encode co-stimulatory domains, such as the CD28 endodomain.

Alternative codons may be used in one or more domains which transmit survival signals, such as OX40 and 41BB endodomains.

Alternative codons may be used in the portions of nucleic acid encoding a CD3zeta endodomain and/or the portions of nucleic acid encoding one or more costimulatory domain(s) and/or the portions of nucleic acid encoding one or more domain(s) which transmit survival signals.

### Vectors

The present disclosure also provides a vector, or kit of vectors which comprises one or more CAR-encoding nucleic acid(s). Such a vector may be used to introduce the nucleic acid(s) into a host cell so that it expresses the first and second CARs.

The vector may, for example, be a plasmid or a viral vector, such as a retroviral vector or a lentiviral vector, or a transposon-based vector or synthetic mRNA.

The vector may be capable of transfecting or transducing a T cell.

### Cells

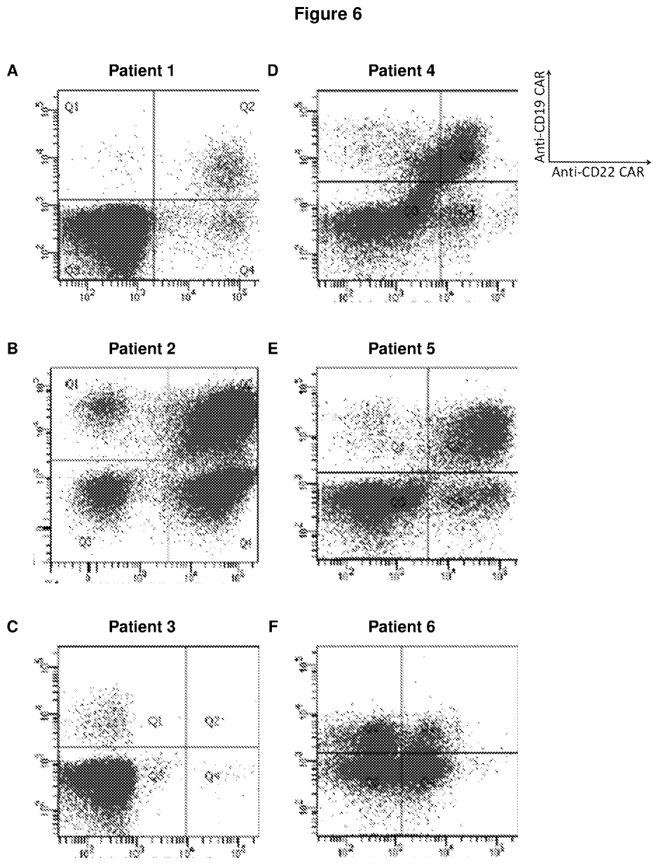

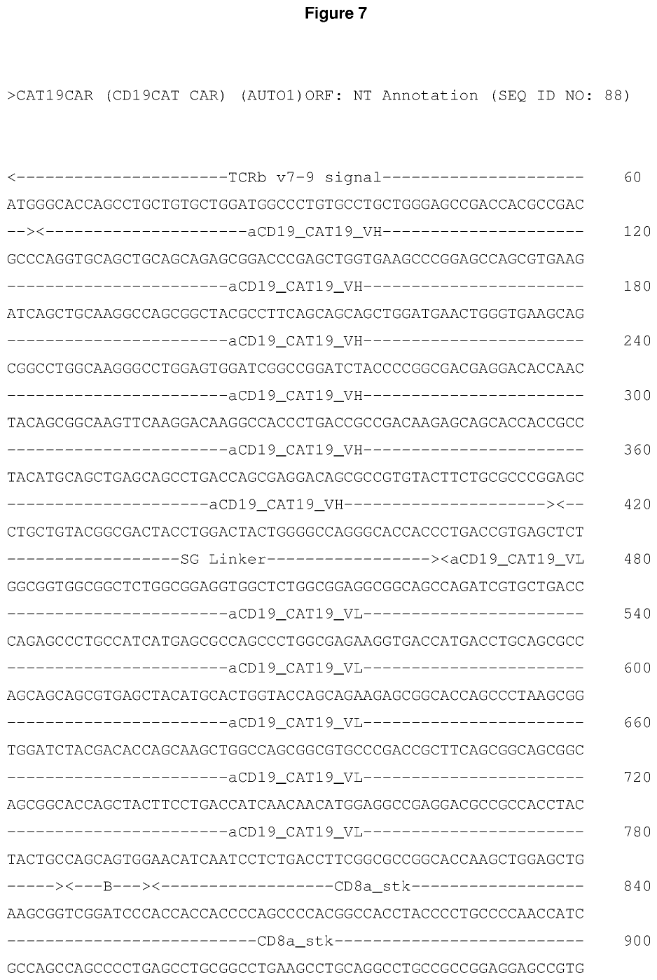

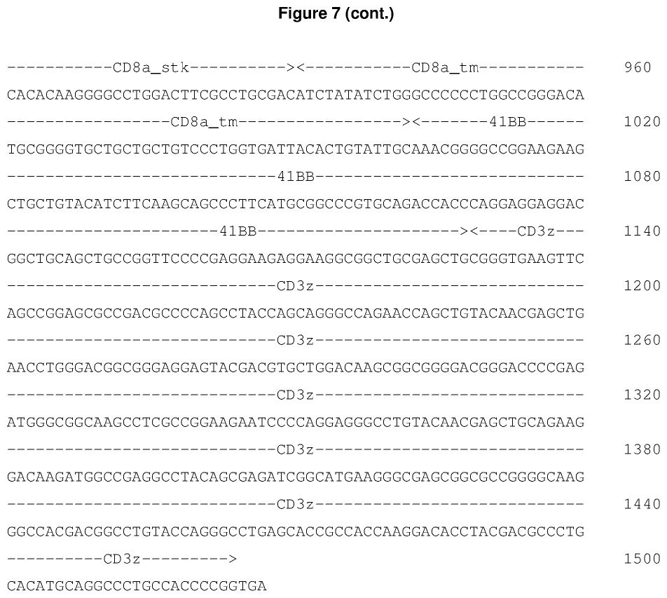

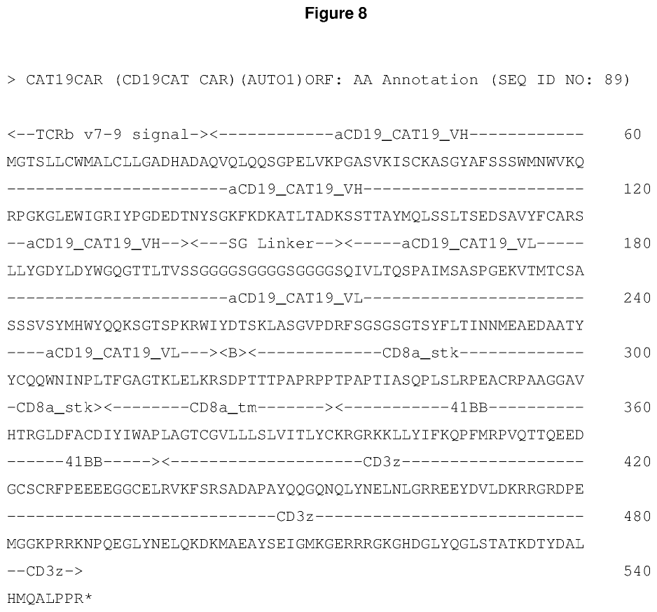

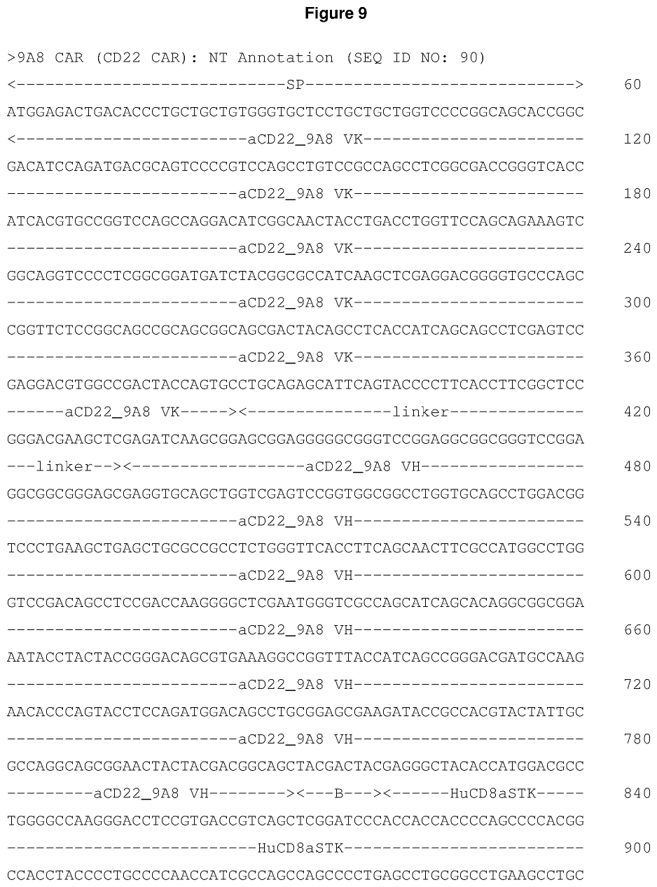

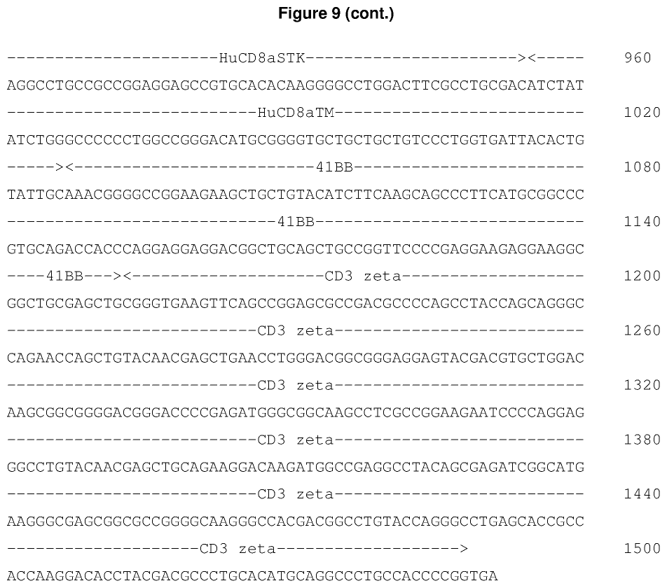

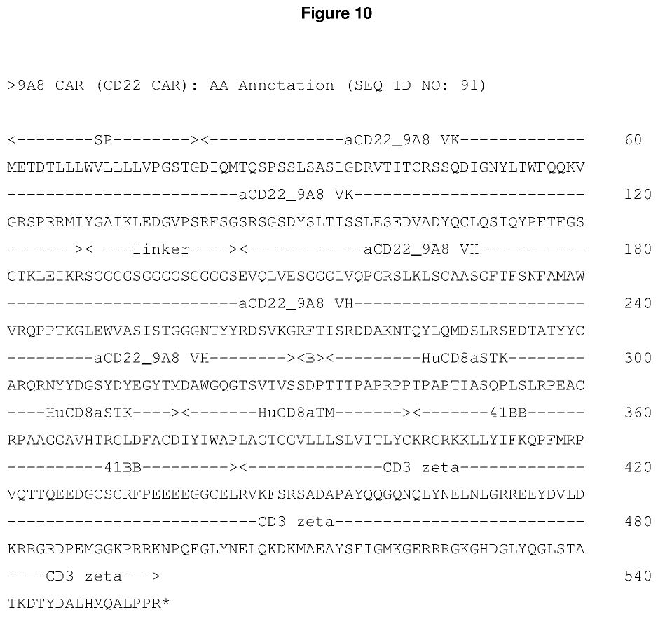

Cells are provided herein which co-express a first CAR and a second CAR, wherein one CAR binds CD19 and the other CAR binds CD22, such that the cell recognizes a target cell expressing either of these markers. Populations of cells which comprise cells which co-express a CD19 CAR and a CD22 CAR, as well as cells that express the CD19 CAR and cells that express the CD22 CAR are also provided. Double transduction has several advantages.
1. Relative effects on persistence can be studied. CAT19 CAR T-cell persistence is well demonstrated. Reported CD22 CAR T-cell persistence is typically short-lived. This may be due to intrinsic properties of CD22 CARs currently under clinical evaluation due to e.g. the short linker used in M971 CARs. Alternatively, this may also be due to reduced signaling due to lower CD22 target density or other factors. Studying the long-term engraftment of single/double-positive populations will help elucidate this. For instance, long-term engraftment of only single-positive CD19 CAR T-cells suggests an intrinsic effect of a CAR; long-term engraftment of only CD19 CAR T-cells (both single and double) would suggest that higher antigen targeting is needed for persistence.
2. Effects of different expression or stoichiometry can be studied. If different relative expression of CD19 vs CD22 CAR are required for optimal persistence, an optimal ratio of expression or co-expression can be elucidated by measuring the CAR expression on long-term engrafted cells.
3. Immune response against the transgene products may be reduced. If two potentially immunogenic binders are encoded in the same expression cassette, the probability of triggering and immune response doubles. With double transduction the probability that at least one population will persist increases. This is observed in patient 3, where the CD22 CAR expressing cells are lost (FIG. 6C).

In some embodiments, populations of cells which comprise cells that express the CD19 CAR and cells that express the CD22 CAR are also provided.

The cell may be any eukaryotic cell capable of expressing a CAR at the cell surface, such as an immunological cell.

In particular, the cell may be an immune effector cell such as a T cell or a natural killer (NK) cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarized below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytotoxic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+ FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr cells or Th3 cells) may originate during a normal immune response.

The T cell provided herein may be any of the T cell types mentioned above, in particular a CTL.

Natural killer (NK) cells are a type of cytolytic cell which forms part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR-expressing cells provided herein may be any of the cell types mentioned above.

CAR-expressing cells, such as CAR-expressing T or NK cells may either be created ex vivo either from a patient's own peripheral blood (1st party), or in the setting of a haematopoietic stem cell transplant from donor peripheral blood (2nd party), or peripheral blood from an unconnected donor (3rd party).

The present disclosure also provides a cell composition comprising CAR-expressing T cells and/or CAR-expressing NK cells, which cells co-express a CAR that binds CD19 and another CAR that binds CD22, such that the cells can recognize a target cell expressing either of these markers. In some embodiments, the cell composition comprises cells that express only a CAR that binds CD19 and cells that express only another CAR that binds CD22. The cell composition may be made by transducing a blood-sample ex vivo with a nucleic acid according to the present disclosure.

The term “CD19/22 CAR T-cells” refers herein to a cell composition comprising a mixture of untransduced cells, cells expressing a CD19 CAR alone, cells expressing a CD22 CAR alone, and cells expressing both the CD19 and CD22 CARs. In some embodiments, the cell composition comprises a mixture of untransduced cells, cells expressing a CD19 CAR alone, and cells expressing a CD22 CAR alone.

Alternatively, T or NK cells provided herein may be derived from ex vivo differentiation of inducible progenitor cells or embryonic progenitor cells to T or NK cells. Alternatively, an immortalized T-cell line which retains its lytic function and could act as a therapeutic may be used.

The CAR cells are generated by introducing DNA or RNA coding for the CARs by one of many means including, but not limited to, transduction with a viral vector, transfection with DNA or RNA. Cells may be activated and/or expanded prior to being transduced with CAR-encoding nucleic acid, for example by treatment with an anti-CD3 monoclonal antibody.

The T or NK cells provided herein may be made by: (i) isolation of a T or NK cell-containing sample from a subject or other sources listed above, and (ii) transduction or transfection of the T or NK cells with one or more a nucleic acid(s) encoding the CD19 and CD22 CARs.

The T or NK cells may then by purified, for example, selected on the basis of expression of the antigen-binding domain of the antigen-binding polypeptide.

### Pharmaceutical Compositions

The present disclosure also relates to a pharmaceutical composition containing a plurality of CAR-expressing cells, such as T cells or NK cells provided herein. Pharmaceutical compositions comprising the CD19/22 CAR T-cell product described in Example 1 are provided. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Methods of Treatment

The present disclosure also relates to methods for treating high risk/relapsed CD19+ or CD22+ haematological malignancy in a patient comprising administering to the patient autologous CD19/22 CAR T-cells (for example, the autologous CD19/22 CAR T-cell product comprising CATi9CAR and 9A8CAR CARs described in Example 1 herein). The present disclosure also relates to autologous CD19/22 CAR T-cells (for example, the autologous CD19/22 CAR T-cell product comprising CATi9CAR and 9A8CAR CARs described in Example 1 herein), or a pharmaceutical composition comprising these cells, for use in the treatment of high risk/relapsed CD19+ or CD22+ haematological malignancy. The present disclosure also relates to the use of autologous CD19/22 CAR T-cells (for example, the autologous CD19/22 CAR T-cell product comprising CATi9CAR and 9A8CAR CARs described in Example 1 herein), or a pharmaceutical composition comprising these cells, in the manufacture of a medicament for the treatment of high risk/relapsed CD19+ or CD22+ haematological malignancy.

The cell compositions of the present disclosure, for example the CD19/22 CAR T-cell product composition described in Example 1, are capable of killing cancer cells recognizable by expression of CD19 or CD22, such as B-cell lymphoma cells. CAR-expressing cells, such as T cells, may either be created ex vivo either from a patient's own peripheral blood (1st party), or in the setting of a haematopoietic stem cell transplant from donor peripheral blood (2nd party), or peripheral blood from an unconnected donor (3rd party). Alternatively, CAR T-cells may be derived from ex-vivo differentiation of inducible progenitor cells or embryonic progenitor cells to T-cells. In these instances, CAR T-cells are generated by introducing DNA or RNA coding for the CAR by one of many means including transduction with a viral vector, transfection with DNA or RNA.

Examples of cancers which express CD19 or CD22 are B-cell lymphomas, including Hodgkin's lymphoma and non-Hodgkins lymphoma; and B-cell leukaemias.

For example the B-cell lymphoma may be Diffuse large B cell lymphoma (DLBCL), Follicular lymphoma, Marginal zone lymphoma (MZL) or Mucosa-Associated Lymphatic Tissue lymphoma (MALT), Small cell lymphocytic lymphoma (overlaps with Chronic lymphocytic leukemia), Mantle cell lymphoma (MCL), Burkitt lymphoma, Primary mediastinal (thymic) large B-cell lymphoma, Lymphoplasmacytic lymphoma (may manifest as Waldenstrom macroglobulinemia), Nodal marginal zone B cell lymphoma (NMZL), Splenic marginal zone lymphoma (SMZL), Intravascular large B-cell lymphoma, Primary effusion lymphoma, Lymphomatoid granulomatosis, T cell/histiocyte-rich large B-cell lymphoma or Primary central nervous system lymphoma.

The B-cell leukaemia may be acute lymphoblastic leukaemia, B-cell chronic lymphocytic leukaemia, B-cell prolymphocytic leukaemia, precursor B lymphoblastic leukaemia or hairy cell leukaemia.

The B-cell leukaemia may be acute lymphoblastic leukaemia (B-ALL or ALL).

The B-ALL may be pediatric ALL (pALL).

The pALL may express CD19 or CD22. The pALL may express CD19 and CD22.

Standard treatment for relapsed pALL includes the following treatment phases: Induction, Consolidation, Interim Maintenance, Delayed Intensification, and Maintenance.

Patients are stratified according to risk levels. Several criteria are available to stratify patients, for example the National Cancer Institute (NCI) criteria, which differentiates patients between NCI Standard Risk and NCI High Risk: NCI Standard Risk patients are patients aged ≥1 year and <10 years old at diagnosis and with a highest white cell count (WCC) before starting treatment of <50×10⁹/L; NCI High Risk patients are patients aged ≥10 years old at diagnosis, and/or with a diagnostic WCC of 250×10⁹/L.

There are national guidelines describing the standard treatment regimen. An example of these guidelines is the UKALL 2019 Interim Guidelines, which is the guideline for the management of ALL in children and young adults used in the UK (also referred to as UKALL 2019 Guidelines). Induction therapy according to the UKALL 2019 Guidelines is as follows
NCI Standard Risk: Patients in this group receive a 3-drug (dexamethasone, vincristine and asparaginase) induction (Regimen A Induction, Table 7).
NCI High Risk: Patients in this group receive a 4-drug (dexamethasone, vincristine, asparaginase and daunorubicin) induction (Regimen B Induction, Table 8).
Regimen C Induction (Table 9): Patients with NCI Standard Risk BCP ALL who are subsequently found to have high risk cytogenetics, or Down syndrome patients with a slow early response.

TABLE 7
Regimen A Induction

| Fluids | All patients should be adequately hydrated (at least 2-2.5 | | | | | | | |
|---|---|---|---|---|---|---|---|---|
| | L/m2/24 h). Given parenterally for the first 48 h. | | | | | | | |
| Allopurinol | 100 mg/m2 oral, three times daily. Should be started 24 h before | | | | | | | |
| | chemotherapy and continue for 5 days. NB. Maximum | | | | | | | |
| | recommended dose in children <15 years old is 400 mg/day. | | | | | | | |
| | Alternative therapy can be used according to local practice. | | | | | | | |
| Dexamethasone | All patients should receive dexamethasone starting on day 1. | | | | | | | |
| | Oral dexamethasone 6 mg/m2/day (maximum dose 10 mg/day in | | | | | | | |
| | induction only) for 28 days starting on day 1 and then tapered | | | | | | | |
| | over the next 7 days. | | | | | | | |
| | The steroid should be divided into two doses per day. | | | | | | | |
| | NB. For severely ill patients, it is permissible to use intravenous | | | | | | | |
| | dexamethasone. | | | | | | | |
| Vincristine | 1.5 mg/m2 (maximum single dose 2 mg) intravenous weekly for five | | | | | | | |
| | weeks starting on day 2 and continuing on days 9, 16, 23 and 30. | | | | | | | |
| Pegaspargase | 1000 iu/m2 intramuscular on day 4 and 18. | | | | | | | |
| (Oncaspar) | | | | | | | | |
| Intrathecal | On days 1, 8 and 29. | | | | | | | |
| methotrexate | Dose by age - <2 yrs: 8 mg; 2 yrs: 10 mg; ≥3 yrs: 12 mg. | | | | | | | |
| | NB. Patients who have CNS disease at presentation should | | | | | | | |
| | receive weekly doses until two clear CSF samples are obtained | | | | | | | |
| | (see section 7.4.1, UKALL 2019 Guidelines). | | | | | | | |
| | Vincristine not to be scheduled on the same day as the | | | | | | | |
| | intrathecal methotrexate. + Regimen A patients can receive | | | | | | | |
| | day 8 intrathecal methotrexate with a day 15 bone marrow, | | | | | | | |
| | if appropriate. | | | | | | | |
| Mercaptopurine | 75 mg/m2/day orally once a day starting on day 29 (beginning | | | | | | | |
| | week 5) (if neutrophils >0.75 × 10\hat{ }9/L and platelets >75 × 10\hat{ }9/L) | | | | | | | |
| | and continuing to day 21 of consolidation (4 weeks from the start | | | | | | | |
| | in week 5 of induction). If necessary, give extra doses between | | | | | | | |
| | induction and consolidation to ensure continuity of therapy. | | | | | | | |
| | Dose adjustments are described in section 8.8.1 (UKALL 2019 | | | | | | | |
| | Guidelines). | | | | | | | |
| Co-trimoxazole | This drug is given as PCP prophylaxis orally twice a day (bd) on 2 | | | | | | | |
| (trimethoprim and | consecutive days each week starting from day 1. Dose for | | | | | | | |
| sulphamethoxazole) | children is based on surface area as detailed below: | | | | | | | |
| | Surface area | | Co-trimoxazole | | Trimethoprim | | Sulphamethoxazole | |
| | 0.5-0.75 | m2 | 240 | mg bd | 40 | mg bd | 200 | mg bd |
| | 0.76-1.0 | m2 | 360 | mg bd | 60 | mg bd | 300 | mg bd |
| | 1.1 m2-1.5 | m2″ | 480 | mg bd | 80 | mg bd | 400 | mg bd |

| | | | | | | |
|---|---|---|---|---|---|---|
| *Over 1.5 m2 | 480-960 | mg | 80-120 | mg bd | 400-800 | mg bd |

*according to local policy over 1.5 m2
See also section 8.8.2 for details of alternative PCP prophylaxis
regimens and permitted dose modifications for toxicity.

TABLE 8
Regimen B Induction

| Fluids | All patients should be adequately hydrated (at least 2-2.5 | | | | | | | |
|---|---|---|---|---|---|---|---|---|
| | L/m2/24 h). Given parenterally for the first 48 h. | | | | | | | |
| Allopurinol | 100 mg/m2 oral, three times daily. Should be started 24 h before | | | | | | | |
| | chemotherapy and continue for 5 days. NB. Maximum | | | | | | | |
| | recommended dose in children <15 years old is 400 mg/day. | | | | | | | |
| | Patients with very high white blood cell count (>100 × 109/L), LBL | | | | | | | |
| | or bulky T-cell disease are at risk of tumour lysis syndrome and | | | | | | | |
| | rasburicase should be considered as an alternative in these | | | | | | | |
| | patients. | | | | | | | |
| Dexamethasone | All patients should receive dexamethasone starting on day 1. | | | | | | | |
| | Oral dexamethasone 6 mg/m2/day (maximum dose 10 mg/day in | | | | | | | |
| | induction only) for 28 days starting on day 1 and then tapered | | | | | | | |
| | over the next 7 days. | | | | | | | |
| | The steroid should be divided into two doses per day. | | | | | | | |
| Daunorubicin | 25 mg/m2 intravenous over 1 hour (or as a bolus alongside a fast | | | | | | | |
| | running infusion in adults) on days 2, 9, 16 and 23. | | | | | | | |
| Vincristine | 1.5 mg/m2 (maximum single dose 2 mg) intravenous weekly for five | | | | | | | |
| | weeks starting on day 2 and continuing on days 9, 16, 23 and 30. | | | | | | | |
| Pegaspargase (Oncaspar) | 1000 iu/m2 intramuscular on day 4 and 18. | | | | | | | |
| Intrathecal methotrexate | On days 1, 8 and 29. | | | | | | | |
| | Dose by age - <2 yrs: 8 mg; 2 yrs: 10 mg; ≥3 yrs: 12 mg. | | | | | | | |
| | NB. Patients who have CNS disease at presentation should | | | | | | | |
| | receive weekly doses until two clear CSF samples are obtained | | | | | | | |
| | (see section 7.4.1, UKALL 2019 Guidelines). | | | | | | | |
| | Vincristine not to be scheduled on the same day as the | | | | | | | |
| | intrathecal methotrexate. | | | | | | | |
| Mercaptopurine | 60 mg/m2/day orally once a day starting on day 29 (beginning | | | | | | | |
| | week 5) (if neutrophils >0.75 × 10\hat{ }9/L and platelets >75 × 10\hat{ }9/L) | | | | | | | |
| | and continuing to day 28 of consolidation (5 weeks from the start | | | | | | | |
| | in week 5 of induction). If necessary, give extra doses between | | | | | | | |
| | induction and consolidation to ensure continuity of therapy. | | | | | | | |
| | Dose not be adjusted according to blood count. | | | | | | | |
| | NB: the mercaptopurine dose in Regimen B induction is different | | | | | | | |
| | to that given in Regimen A. | | | | | | | |
| Co-trimoxazole | This drug is given as PCP prophylaxis orally twice a day (bd) on 2 | | | | | | | |
| (trimethoprim and | consecutive days each week starting from day 1. Dose for | | | | | | | |
| sulphamethoxazole) | children is based on surface area as detailed below: | | | | | | | |
| | Surface area | | Co-trimoxazole | | Trimethoprim | | Sulphamethoxazole | |
| | 0.5-0.75 | m2 | 240 | mg bd | 40 | mg bd | 200 | mg bd |
| | 0.76-1.0 | m2 | 360 | mg bd | 60 | mg bd | 300 | mg bd |
| | 1.1 m2-1.5 | m2″ | 480 | mg bd | 80 | mg bd | 400 | mg bd |

| | | | | | | |
|---|---|---|---|---|---|---|
| *Over 1.5 m2 | 480-960 | mg | 80-120 | mg bd | 400-800 | mg bd |

*according to local policy over 1.5 m2
See also section 8.8.2 for details of alternative PCP prophylaxis
regimens and permitted dose modifications for toxicity.

TABLE 9
Regimen C Induction

| Dexamethasone | All patients should receive dexamethasone starting on day 1. | | | | | | | |
|---|---|---|---|---|---|---|---|---|
| | Oral dexamethasone 6 mg/m2/day (maximum dose 10 mg/day in | | | | | | | |
| | induction only) for a total of 28 days from commencing on | | | | | | | |
| | regimen A and then tapered over the next 7 days i.e. to stop on | | | | | | | |
| | day 35. The steroid should be divided into two doses per day. | | | | | | | |
| | Down Syndrome patients aged >10 years: receive oral | | | | | | | |
| | dexamethasone 10 mg/m2/day for 7 days starting on day 15. The | | | | | | | |
| | steroid should be divided into two doses per day. Do not cap | | | | | | | |
| | dexamethasone dose. | | | | | | | |
| Vincristine | 1.5 mg/m2 (maximum single dose 2 mg) intravenous weekly on | | | | | | | |
| | days 16, 23 and 30 | | | | | | | |
| Daunorubicin | 45 mg/m2 intravenous over 1 hour (or as a bolus alongside a fast | | | | | | | |
| | running infusion in adults) on days 16 and 23. Note that this dose | | | | | | | |
| | is higher than that in regimen B and is for two doses only. | | | | | | | |
| Pegaspargase (Oncaspar) | 1000 iu/m2 intramuscular on day 18. | | | | | | | |
| Intrathecal methotrexate | On day 29. Dose by age - <2 yrs: 8 mg; 2 yrs: 10 mg; ≥3 yrs: 12 mg. | | | | | | | |
| | NB. Patients who have CNS disease at presentation should | | | | | | | |
| | receive weekly doses until two clear CSF samples are obtained | | | | | | | |
| | (see section 7.4.1, UKALL 2019 Guidelines). | | | | | | | |
| | Vincristine not to be scheduled on the same day as the | | | | | | | |
| | intrathecal methotrexate. | | | | | | | |
| Mercaptopurine | 60 mg/m2/day orally once a day starting on day 29 (beginning | | | | | | | |
| | week 5) (if neutrophils >0.75 × 10\hat{ }9/L and platelets >75 × 10\hat{ }9/L) | | | | | | | |
| | and continuing to day 14 of consolidation (3 weeks from the start | | | | | | | |
| | in week 5 of induction). If necessary, give extra doses between | | | | | | | |
| | induction and consolidation to ensure continuity of therapy. | | | | | | | |
| | Dose not be adjusted according to blood count. | | | | | | | |
| | NB: the mercaptopurine dose in Regimen C induction is different | | | | | | | |
| | to that given in Regimen A. | | | | | | | |
| Co-trimoxazole | This drug is given as PCP prophylaxis orally twice a day (bd) on 2 | | | | | | | |
| (trimethoprim and | consecutive days each week starting from day 1. Dose for | | | | | | | |
| sulphamethoxazole) | children is based on surface area as detailed below: | | | | | | | |
| | Surface area | | Co-trimoxazole | | Trimethoprim | | Sulphamethoxazole | |
| | 0.5-0.75 | m2 | 240 | mg bd | 40 | mg bd | 200 | mg bd |
| | 0.76-1.0 | m2 | 360 | mg bd | 60 | mg bd | 300 | mg bd |
| | 1.1 m2-1.5 | m2″ | 480 | mg bd | 80 | mg bd | 400 | mg bd |

| | | | | | | |
|---|---|---|---|---|---|---|
| *Over 1.5 m2 | 480-960 | mg | 80-120 | mg bd | 400-800 | mg bd |

*according to local policy over 1.5 m2
See also section 8.8.2 for details of alternative PCP prophylaxis
regimens and permitted dose modifications for toxicity.

Details of the treatment regiments of the Consolidation, Interim Maintenance, Delayed Intensification, and Maintenance phases can be found on the UKALL 2019 Guidelines (docplayer.net/170103468-CIcn-ukall-2019-interim-guidelines.html, which are included herein by reference).

Treatment with the T cells provided herein is contemplated to help prevent the escape or release of tumor cells which often occurs with standard care approaches.

The methods, autologous CD19/22 CAR T-cells, or uses provided herein slow or prevent progression of the cancer, diminish the extent of the cancer, result in remission (partial or total) of the cancer, and/or prolong survival of the patient.

In the provided methods, autologous CD19/22 CAR T-cells, or uses, the patient treated has a high risk/relapsed or resistant CD19+ or CD22+ haematological malignancy.

Where the CD19+ or CD22+ haematological malignancy is pALL, there are several parameters that may be used to define high risk/relapsed or resistant pALL:
a) resistant disease,
b) ALL with persisting high level minimal residual disease (MRD) at 2nd time point of frontline national protocol,
c) high risk infant ALL
d) intermediate risk infant ALL,
e) high risk first relapse,
f) standard risk relapse in patients with high-risk cytogenetics,
g) standard risk relapse with bone marrow minimal residual disease (MRD)>10⁻³ at end of re-induction,
h) any refractory relapse of ALL, or
i) any relapse of CD22+ lymphoma.

Resistant disease is defined as the presence of >5% blasts at the end of the induction phase according to UKALL 2019 guidelines or equivalent induction. This defines the primary refractory population.

ALL with persisting high level minimal residual disease (MRD) at 2nd time point of frontline national protocol is defined as MRD>10⁻⁴ at week 14 according to UKALL 2019 guidelines or equivalent.

High risk infant ALL is defined as an infant of age <6 months at diagnosis with MLL gene rearrangement and either presenting white cell count >300×10⁹/L or poor steroid early response. Poor steroid early response is defined as the presence of circulating blast count >1×10⁹/L following 7 day steroid pre-phase of induction as per national guidelines (e.g., UKALL 2019 Interim Guidelines or equivalent).

Intermediate risk infant having ALL is defined as an infant presenting with MRD >10-3 at end of induction as per national guidelines (e.g. UKALL 2019 Interim Guidelines or equivalent).

High risk first relapse is defined as a bone marrow relapse or isolated/combined extramedullary relapse within thirty months of diagnosis, as per the updated INTREALL 2010 classification [International Study for Treatment of High Risk Childhood Relapsed ALL (IntReALL) HR 2010 study; NCT03590171]

High risk cytogenetics is defined as cytogenetic abnormalities that have correlated with a poor outcome, and include:
Philadelphia chromosome or Philadelphia translocation (Ph), also termed BCR-ABL1 or t(9; 22)(q34; q11),
BCR-ABL1-like or Philadelphia-like (Ph-like), such as, ABL-class fusions, which involve the fusion of a kinase gene (e.g. ABL1, ABL2, CSF1R, PDGFRA or PDGFRB) to wide variety of “activating genes” (e.g. ETV6, PAX5, EBF1, NUP214, ZMIZ1, FLIPLi, et cetera),
MLL (KMT2A) rearrangement,
near-haploidy (<30 chromosomes) and low hypodiploidy (30-39 chromosomes),
Intrachromosomal amplification of chromosome 21 (iAMP21), and
TCF3-HLF translocations, i.e. t(17; 19)(q22; p13)/TCF3(E2A)-HLF), which is a variant of the t(1; 19)(E2A-PBX), t(17; 19)(E2A-HLF).

Standard risk relapse is defined as presenting bone marrow minimal residual disease (MRD)>10⁻³ at end of re-induction,

Any refractory relapse of ALL is defined as >1% blasts by flow cytometry after at least one cycle of standard chemotherapy.

The patient may be ineligible for treatment with other CD19 CAR T-cell products, such as Kymriah™ (Tisagenlecleucel).

The patient may have received one or more lines of prior therapy. The patient may have received two or more, three or more, four or more, five or more, or six or more lines of prior therapy.

The patient may have received a prior CD19 immunotherapeutic treatment. Examples of anti-CD19 immunotherapeutic therapies include, without limitation, inotuzumab ozogamicin (Besponsa®), blinatumomab (Blincyto®) and Tisagenlecleucel (Kymriah™). The patient may have previously been administered one or more of inotuzumab ozogamicin (Besponsa®), blinatumomab (Blincyto®) and Tisagenlecleucel (Kymriah™). The patient may have previously been administered one or more of inotuzumab ozogamicin (Besponsa®), blinatumomab (Blincyto®) and tisagenlecleucel (Kymriah™).

The patient may present extramedullary disease.

The patient may have received allogeneic stem cell transplant.

The patient may be administered a single dose of 1×10⁶ CAR T-cells/kg, such as CD19/22 CAR T-cell product described in Example 1. The patient may be administered a single dose of 0.5×10⁶ CAR T-cells/kg, such as the CD19/22 CAR T-cell product described in Example 1. The patient may be administered a single dose of 0.75×10⁶ CAR T-cells/kg, such as the CD19/22 CAR T-cell product described in Example 1. The patient may be administered a single dose of 1.2×10⁶ CAR T-cells/kg, such as CD19/22 CAR T-cell product described in Example 1. The administration may be an intravenous injection, for example through a Hickman line or peripherally inserted central catheter (PICC line).

The patient may show progression-free survival of at least six months after said administration, or at least twelve months after said administration.

### Other Terminology and Disclosure

As used herein and in the appended claims, the singular forms “a,” “and,” and “the” include plural referents unless the context clearly dictates otherwise. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

When a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of the present disclosure.

All publications mentioned herein are incorporated herein by reference to disclose and describe the methods and/or materials for the purpose for which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This disclosure is intended to provide support for all such combinations.

As used herein, “may,” “may comprise,” “may be,” “can,” “can comprise” and “can be” all indicate something envisaged by the inventors that is functional and available as part of the subject matter provided.

Examples While the following examples describe specific embodiments, variations and modifications will occur to those skilled in the art. Accordingly, only such limitations as appear in the claims should be placed on the invention.

### EXAMPLE 1

Generation of a CAR-T Cell Composition Transduced with Multiple Vectors

Lentiviral vectors were generated expressing either a) a second-generation CD19 CAR (SEQ ID NO: 89)(CD19CAT CAR described in WO2016/139487, otherwise referred to herein as CAT CAR or AUTO1) which comprises an anti-CD19 antigen-binding domain, a CD8 stalk spacer and transmembrane domain, and a compound 4-1BB-CD3 endodomain, under the control of a PGK promoter (pCCL.PGK.aCD19cat-CD8STK-41BBZ); or b) a CD22 CAR (SEQ ID NO: 91)(9A8-1 based CAR described in WO2019/220109)(9A8 CAR) which comprises an anti-CD22 antigen-binding domain, a CD8 stalk spacer and a second generation endodomain comprising CD3 and a 4-1BB costimulatory domain, under the control of an EF1a promoter (pCCL.EF1a.aCD22_9A8-1-64_LH_scFv-CDBSTK-41BBz). See FIG. 1A-B.

Two separate lentiviral supernatants were produced and mixed 1:1 at an MOI of 2.5+2.5.

Normal donor T cells were transduced with either CAT CAR lentiviral vector, 9A8 CAR lentiviral vector or double-transduced with both. T cells were stained with anti-CAT idiotype (to detect CAT CAR) and recombinant soluble CD22 (to detect 9A8 CAR). At an MOI of 2.5/2.5 of each vector, single-positive and double-positive populations were observed.

Post-transduction with the lentiviral composition, the cells are a mixture of untransduced cells (46.5%); cells expressing the CD19 CAR alone (23.1%); cells expressing the CD22 CAR alone (11.1%) and cells expressing both the CD19 and CD22 CARs (19.3%).

The resulting mixed population is referred to as “CD19/22 CAR T-cell product,” “CD19CAT-CD22 9A8-41BBZ CAR T-cell product” or “AUTO1/22 product” herein.

These experiments demonstrate that it was possible to generate a mixed population of T-cells which include T cells which are single and double-positive for each CAR and that the expression of the CAR was not affected by double transduction.

### EXAMPLE 2

### Pre-Clinical Assessment

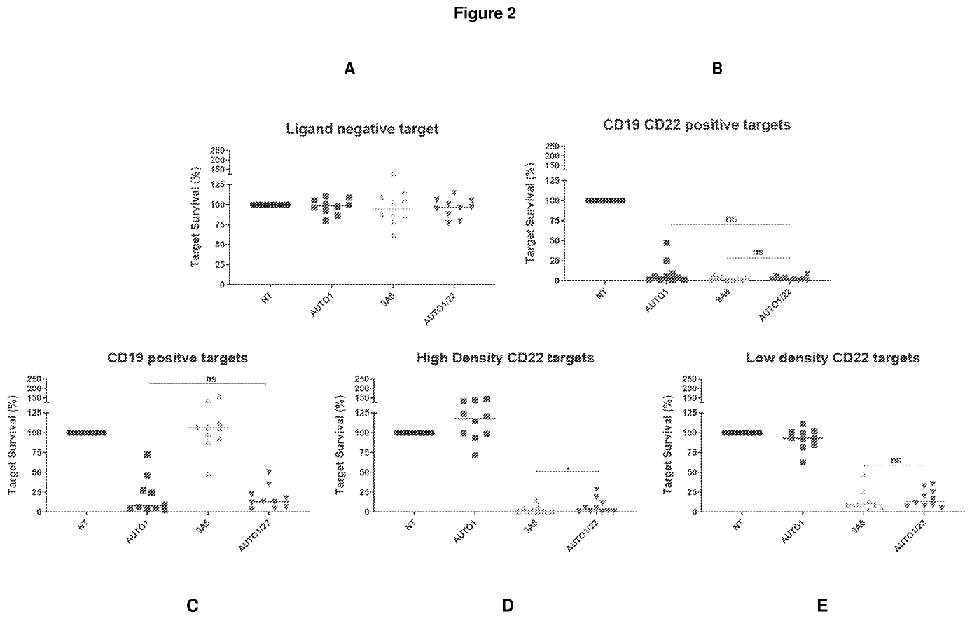

The in vitro functional performance of CD19/22 CAR T-cell product of Example 1 was determined. T cells were challenged with SupT1 cells (which are CD19 and CD22 negative), and SupT1 cells engineered to express either or both CD19 and CD22 [at high or low (<1000 copies of CD22) surface antigen density]. Target cell killing and IFN-gamma and IL-2 cytokine release was measured. The CD19/22 CAR T-cell product maintained cytolytic capacity on all tested targets compared to single CAR expressing T-cells (FIG. 2A-E). Both CD19 CAR T cells and CD19/22 CAR T cells were able to efficiently kill and secrete cytokines in response to targets expressing low levels of CD22.

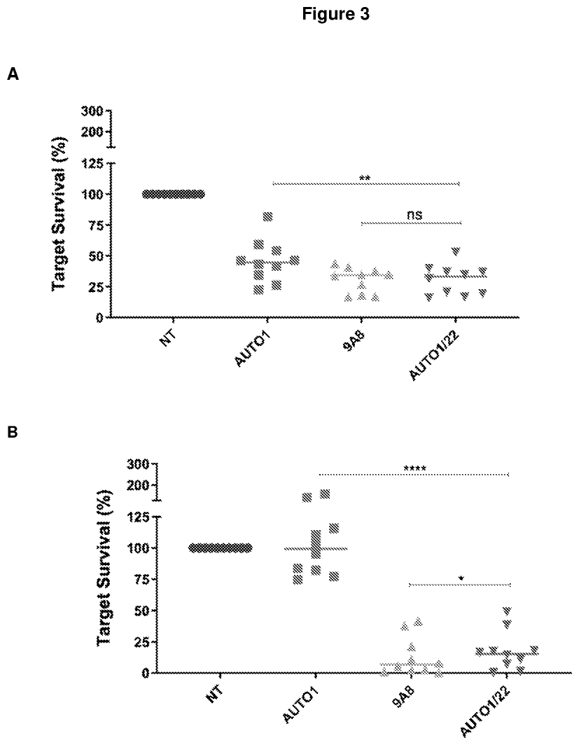

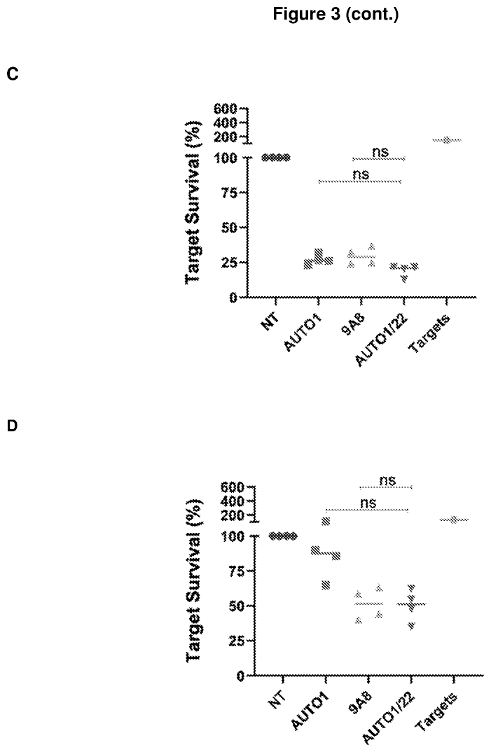

Raji cells are a B cell line derived from Burkitt's lymphoma and natively express both CD19 and CD22. To determine the performance of the CD19/22 CAR T-cell against a target cell with native expression of CD19/CD22 functional tests with normal donor T cells were performed. To simulate the scenario of CD19 negative escape, functional tests were additionally performed with Raji cells with CD19 gene disruption. The CD19/22 CAR T-cell product maintained cytolytic function on a CD19 knock-out Raji cell line and on a CD19 negative primary human B-ALL target cell (FIG. 3A-D).

Next, the function of CD19 CAR T cells (AUTO1) and CD19/22 co-transduced CAR T cells (AUTO1/22) in vivo was determined. NALM6 cells (cell line derived from B-ALL, which expresses both CD19 and CD22) were engineered to express HA-tagged firefly Luciferase. To simulate the scenario of CD19 negative escape, the HA-FLuc NALM6 cell line was further engineered by genome editing to disrupt CD19 expression (NALM6 CD19ko). NALM6 cells were first engrafted in NSG mice by tail-vein injection, and their engraftment determined by bioluminescence imaging (BLI). Following this, equal numbers of either non-transduced, AUTO1 cells or AUTO1/22 CAR T cells were administered by tail vein injection. NALM6 burden was measured sequentially using BLI. At day 14 post CAR T-cell administration, mice were sacrificed, and necropsy performed. NALM6 burden and CAR T-cell engraftment was determined by flow cytometry.

At a suboptimal dose for in vivo tumor clearance, the CD19/22 CAR T-cell product was significantly better at controlling double positive (Nalm-6 WT) tumor growth than CATi9CAR. Only the CD19/22 CAR T-cell product was able to control CD19 negative (Nalm-6 CD19KO) tumor growth. See FIG. 4A-E.

Finally, the proportion of AUTO1, CD22CAR and AUTO1/22 CAR T cells engrafted in mice at sacrifice was determined by flow-cytometry of marrow aspirate. As shown in FIG. 4F, AUTO1/22 CAR T cells maintained their relative proportion after challenge with NALM6 cells. When CAR T cells were challenged with NALM6 CD19 ko cells, CD22CAR+ and AUTO1/22 (CD19CAR/CD22CAR) T cells expanded, while AUTO1 (CD19CAR) single-positive T cells did not expand (FIG. 4F). When challenged with the Nalm-6 CD19KO model, there was selective expansion and enrichment of CAR T-cells expressing the anti-CD22 9A8 CAR within the recipient bone marrow with a loss of single anti-CD19 CAR T-cells.

### EXAMPLE 3

### Clinical Product Phenotype

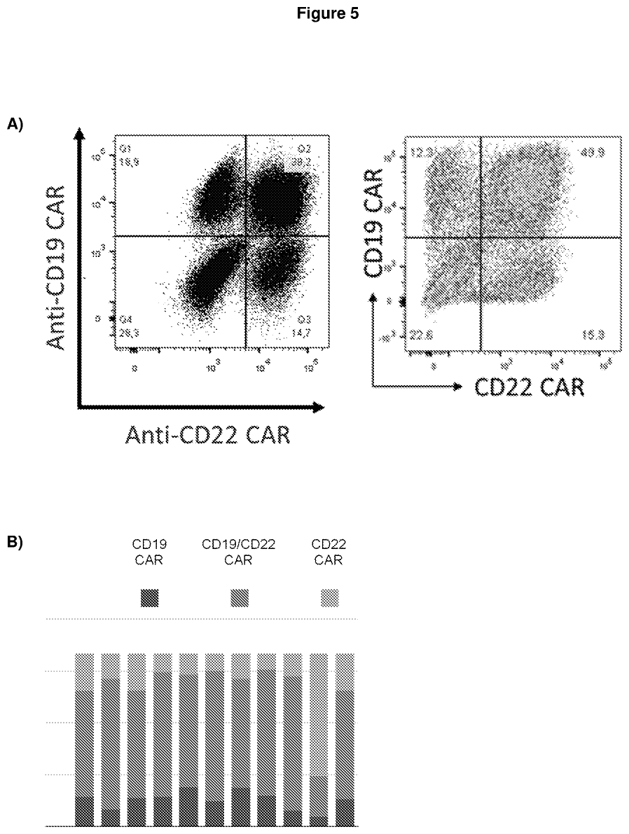

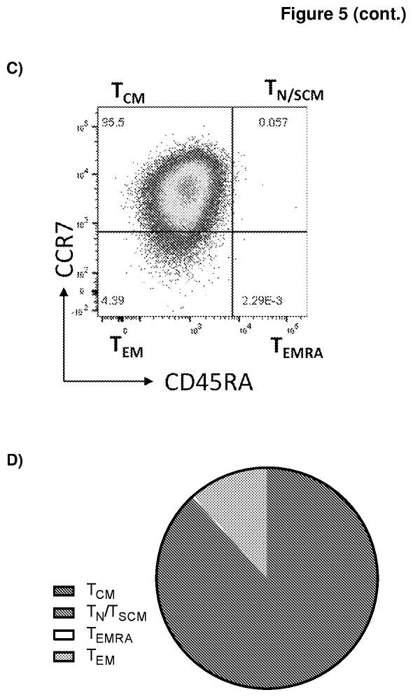

Full scale manufacturing also produced a therapeutic CD19/22 CAR T-cell product with a mixture of single and double CAR positive T cells (FIG. 5A, 5B). Briefly, PBMCs were obtained from fresh or thawed unstimulated leukapheresis (double volume leukapheresis will be performed according to local institutional practice if absolute lymphocyte count >0.5×10⁹/L; for patients with absolute lymphocyte count <0.5×10⁹/L a 2.5 volume leukapheresis will be carried out). PBMCs were CD3/CD28 activated in X-VIVO 15 medium. For patients with absolute lymphocyte count <0.3×10⁹/L, culture medium from this point was supplemented with interleukin-2 (IL-2). Transduction was then carried out in retronectin-coated differentiation bags, by exposure of the activated PBMCs to lentiviral vector at a defined multiplicity of infection (MOI). MOI of 3.5 for the CD19CAR vector and 1.5-2.5 for the CD22CAR vector. These MOIs were selected to give both high level transduction efficiency and balanced populations of CD19 and CD22CAR single positive populations. On day 4, the lentiviral vector was removed by centrifugation and the cells were transferred into a WAVE bioreactor cell culture bags in fresh X-VIVO 15 medium +/−IL-2 (depending on starting absolute lymphocyte count). The cells were then expanded in the WAVE bioreactor for up to a further 3 days. Following this, the CAR transduced T cells were cryopreserved in infusible cryomedia (CryoStor® CS10, cryopreservation medium containing 10% USP grade DMSO. Aliquots taken at this time were then subjected to quality control assays to ensure the transduced T cell product met the release criteria listed further below.

The release criteria for the CAR T cells are:
a) Bacterial/Fungal sterility
b) Absence of Mycoplasma (PCR)
c) Absence of endotoxin (<2EU/ml in Limulus Amoebocyte Lysate assay)
d) Viability of >70% (flow cytometry)
e) Transduction efficiency: 210% CD19CAT-CD22 9A8 CAR+ cells, 22.5% CD19CAT CAR+ cells, 22.5% CD22 9A8 CAR+ cells
f) Cell dose 1.2×10⁶ CAR+ T cells/kg (manual count) at cryopreservation (=10⁶ CAR+ T cells/kg accounting for 20% cell loss during thawing)*
Where the Advanced Therapy Investigational Medicinal Product (ATIMP) meets release criteria but it is not possible to generate this target dose and it is not feasible to generate another dose, a reduced dose (minimum 0.6×10⁶ CAR+ T cells/kg at cryo-preservation=5×10⁵ CAR+ T cells/kg accounting for 20% cell loss during thawing) may be administered.

In addition, the following assays may have been performed, though they do not constitute release criteria: flow cytometry to determine the immunophenotype and the percentage of non-T cell immune sub-sets in the ATIMP, viral copy number assessment by qPCR.

Results shown in FIG. 5A, 5B revealed that the dual transduced population dominated and, in general, the CD19 and CD22 CAR single CAR populations were balanced in the ATIMP. Table 20 shows CAR-T cell dose, transduction (Td) efficiency (expressed as percentage of T cells expressing CD19 single, CD22 single or CD19/22 dual CAR), composition between single CD19, single CD22 and double CD19/22 transduced product in the whole cohort of pts as well as vector copy number. Median, ranges and interquartile ranges (IQR) were reported.

TABLE 20

| | Median | Range |
|---|---|---|
| CAR T cell dose | 1 × 10\hat{ }6/kg | 0.9-1.2 |
| Total CAR Td efficiency | 83.26% | 60.8-92.6% |
| CD19 single CAR Td | 13.10% | 8.2-21.8% |
| CD22 single CAR Td | 11.62% | 8.1-43.1% |
| CD19/22 dual Td | 54.40% | 14.1-70.0% |
| Vector copy number | 5.5 | 3.39-8.00 |

There was no difference in the proportions of naïve, central or effector memory and terminally differentiated T-cells in the different expressing CAR populations of the product. All three CAR T cell populations displayed preservation in early memory T cell phenotypes. See, the following Tables 10 and 11.

TABLE 10

| | | | CAT | 9A8 | CAT/9A8 |
|---|---|---|---|---|---|
| CD8+ Subset | Ctrl. - | NT | CAR | CAR | CAR |
| T_{N} + T_{SCM} | 22.5% | 15.5% | 24.7% | 27.8% | 21.9% |
| T_{CM} | 26.6% | 28.0% | 57.2% | 56.0% | 63.8% |
| T_{PM} | 35.2% | 43.4% | 14.5% | 12.5% | 12.1% |
| T_{EMRA+} | 15.7% | 13.0% | 3.67% | 3.70% | 2.37% |

TABLE 11

| | | | CAT | 9A8 | CAT/9A8 |
|---|---|---|---|---|---|
| CD4+ Subset | Ctrl. - | NT | CAR | CAR | CAR |
| T_{N} + T_{SCM} | 5.89% | 2.34% | 4.01% | 5.34% | 3.91% |
| T_{CM} | 52.3% | 31.9% | 65.4% | 65.0% | 68.6% |
| T_{PM} | 40.2% | 63.6% | 30.2% | 28.6% | 26.5% |
| T_{EMRA+} | 1.16% | 2.22% | 0.44% | 0.99% | 0.99% |

The characteristics of the ATIMP from 11 patients (n=11) are as follows:
Median transduced cell dose achieved: 664×10⁶ (range 66-1597)
Median transduction efficiency was 83% (range 60.8-92.6): CD19/22 DP>CD19SP=CD22SP in product
Median vector copy number (VCN) was 5.5 (range 3.39-8.00)
The proportions of naïve, central or effector memory and terminally differentiated T-cells are: T_{CM} 87.7%, T_{N/SCM} 0.56%, T_{EM} 11.61%, T_{EMRA} 0.17%.

### EXAMPLE 4

### Methods

### Clinical Laboratory Evaluations

Serum cytokine measurements were assessed on days 0, 2, 5, 7, 9, 12, 14 post-CAR T cell infusion by an ISO-accredited method using cytometric bead array analysis of IL-2, IL-4, IL-6, IL-10 TNF, IFNγ (BD Biosciences). The validated lower limit of this assay is 50pg/ml and the upper limit was 5000pg/ml.

CAR T cell expansion/persistence were assessed in the peripheral blood (PB) on days 0, 2, 7, 14, 28, monthly up to 6 months, 6 weekly to 1 year then 3 monthly up to 2 years post infusion. Bone marrow (BM) was assessed monthly for the first 6 months and then at the same intervals as for blood. CAR T cells were detected using a validated qPCR assay detecting a transgene-specific sequence. Mononuclear cells were isolated from peripheral blood and bone marrow and DNA extracted. Transgene-specific primers and probes for the CD19CAR and CD22CAR coding sequences (Integrated DNA Technologies) were utilised in a qPCR reaction plate incorporating a parallel control gene (albumin). Known standards of the target were run for both target and control gene. This provided quantitative data for target and control gene which could be used to quantitate CAR copies per pg/gDNA with a detection limit of 100 copies/pg DNA. Circulating CAR T cells in blood and bone marrow were also analyzed by flow cytometry using an anti-CAT and anti-9A8 CAR anti-idiotype antibodies. Absolute T cell numbers were obtained using a Trucount method (BD Biosciences) and staining for viable, CD45+CD3+ cells. Reagents used were 7-AAD, Fc gamma block, CD45 FITC, CD3 APC-Cy). The percentage of CAR+ T cells was assessed using an anti-CAT CAR anti-idiotype and secondary anti-rat IgG PE antibody and an anti-9A8 CAR anti-idiotype and secondary anti-Rabbit IgG BV421 with co-staining to allow detection of viable CAR+CD45+CD3+ cells. From this, the absolute CAR T cell count was established. Normal donor PBMC were used as negative controls. The threshold for detection was 0.1% CAR T cells.

### Analysis of Cellular Kinetics

Analysis of CAR T cell kinetics was performed on from the CAR transgene. Area under the curve analysis of CAR T cell levels up to 28 days (AUC 0-28) was estimated by a trapezoidal algorithm and represented early CAR T cell expansion. Cmax was the peak concentration of CAR T cells documented, Tmax was the time in days from infusion to maximal CAR T cell concentration, Tlast was the time from infusion to the last documented detection of CAR T cells. T1/2 was the half-life of CAR T cell persistence over the contraction phase, as measured in patients with a minimum of 3 data points documented after Tmax.

### Clinical Study

A study of the safety, efficacy and duration of response to the CD19/22 CAR T-cell product was initiated in children and young adults with high risk, relapsed CD19+ and/or CD22+ haematological malignancies (Acute Lymphoblastic Leukemia and Burkitt's lymphoma). This is Cohort 3 of a multi-centre, non-randomised, open label Phase I clinical trial.

The study design for Cohort 3 is summarized in the following Table 12.

TABLE 12

| Arms | Assigned Interventions |
|---|---|
| Experimental: CD19/22 CAR T-cells | Procedure/Surgery: Leukapheresis |
| Patients meeting the eligibility criteria will have | Patients will undergo an unstimulated |
| leukapheresis to isolate the blood immune cells | leukapheresis to isolate the required immune |
| used to manufacture the CD19/22CAR T-cells. | cells to produce the CD19/22 CAR T-cells |
| Patients will receive lymphodepletion with | Drug: Lymphodepletion with fludarabine |
| fludarabine and cyclophosphamide prior to | Patients will receive lymphodepleting chemotherapy |
| infusion of CD19/22 CAR T-cells | with iv fludarabine 30 mg/m2 on days −7 |
| | to −3 prior to CD19CAR T-cell infusion. |
| | Drug: Lymphodepletion with cyclophosphamide |
| | Patients will receive lymphodepleting chemotherapy |
| | with iv cyclophosphamide 0.5 g/m2 on |
| | days −4 to −2 prior to CD19CAR T-cell infusion |
| | Biological/Vaccine: CD19/22 CAR T-cells |
| | A single dose of 1 × 10\hat{ }6/kg CD19/22CAR |
| | transduced T-cells will be given as an |
| | intravenous injection |
| | through a Hickman line or PICC |
| | line(peripherally inserted central catheter) on |
| | day 0. |

The primary outcome measures for the study were as follows.
a) Toxicity evaluation following CD19/22CAR T-cell infusion
The incidence of grade 3-5 toxicity occurring within 60 days of CD19/22CAR T-cell infusion.

In particular, the incidence of Severe Cytokine Release Syndrome and Grade 3-5 neurotoxicity occurring within 30 days of CD19/22CAR T-cell infusion. [Time Frame: 1 month]
b) Molecular remission
Efficacy was assessed by determining Minimal Residual Disease in the bone marrow aspirate using immunoglobulin heavy chain (IgH) quantitative polymerase chain reaction (qPCR) and/or Next Generation Sequencing in all patients. The proportion of patients achieving molecular remission at 1 month post CD19/22CAR T-cell infusion. [Time Frame: 1 month]

The secondary outcome measures for the study were as follows.
a) Long term molecular remission
Number of patients in molecular remission without further therapy at 2 years. [Time Frame: 2 years]
b) Frequency of circulating CD19/22 CAR T-cells
Persistence and frequency of circulating CD19/22CAR T-cells in the peripheral blood by flow cytometry and qPCR analyses. [Time Frame: 2 years]
c) Incidence of hypogammaglobulinemia
Incidence and duration of hypogammaglobulinemia [Time Frame: 2 years]
d) Relapse rate
Relapse rate monitored during interventional phase and long term follow up for a total of 10 years post cell infusion. Number of patients who relapsed can be summarized as a percentage or rate (for all patients registered to the trial, and also only for those who received the cell infusion). [Time Frame: 10 years]
e) Duration of response.
Duration of response is measured from the time of documented response to the time of molecular or morphological relapse or death, whichever occurs first, with patients who did not experience a disease failure event being censored at the date of their last follow-up.
f) One- and two-year post-infusion event-free survival (EFS).
Event free survival was defined as reported in the ELIANA study where events of interest included no response, morphological relapse before response was maintained for at least 28 days, morphological relapse after having complete remission with or without incomplete hematologic recovery or death, whichever occurred first. Patients were censored if they received further therapy or at the date last seen alive. Event free survival was further defined by a more stringent criteria that included as events the failure to achieve remission, morphological or molecular relapse after remission, or death, whichever occurred first.
g) Overall survival
OS was measured as the time from infusion of CAR T cells to time of death, with patients who did not experience the event of interest being censored at the day they were last seen alive. Overall survival is monitored during interventional phase and long term follow up for 10 years post-CD19/22 CAR T-cell infusion. Number of patients alive can be summarized as a percentage (for all patients registered to the trial, and also only for those who received the cell infusion). [Time Frame: 10 years]

Eligible patients were children and young adults (age ≤24 years) with high risk, relapsed CD19+ and/or CD22+ B lineage ALL and ineligible for Kymriah on the UK national access program.

The inclusion criteria for the study were as follows.
Children and young adults (age 24 years or younger) with high risk/relapsed CD19+ and/or CD22+ haematological malignancy who have:
a) Resistant disease (>5% blasts) at end of UKALL 2019 guidelines or equivalent induction
b) ALL with persisting high level MRD at 2nd time point of frontline national protocol (currently MRD>10⁻⁴ at week 14 UKALL 2019 guidelines or equivalent).
c) High risk infant ALL (age <6 months at diagnosis with MLL gene rearrangement and either presenting white cell count >300×10⁹/L or poor steroid early response (i.e. circulating blast count >1×10⁹/L following 7 day steroid pre-phase of induction as per national guidelines or equivalent)
d) Intermediate risk infant ALL with MRD>10⁻³ at end of induction following national guidelines or equivalent)
e) High risk 1st relapse (as defined by updated IntreALL 2019 classification: bone marrow or combined relapse within 30 months of diagnosis OR any relapse within 18 months of diagnosis)
f) Standard risk relapse in patients with high-risk cytogenetics (defined as BCR-ABL, KMT2A rearrangement, near-haploidy (<30 chromosomes) and low hypodiploidy (30-39 chromosomes), iAMP21 and TCF3-HLF translocations).
g) Standard risk relapse with bone marrow minimal residual disease (MRD)>10⁻³ at end of re-induction
h) Any on therapy relapse in patients age 16-24
i) Any relapse of infant ALL
j) ALL post a 2nd relapse
k) Any refractory relapse of ALL (defined as >1% blasts by flow cytometry after a at least 1 cycle of standard chemotherapy)
l) ALL with MRD>10⁻⁴ prior to planned stem cell transplant
m) Any relapse of ALL eligible for stem cell transplant but no available HLA matched donor or other contraindication to transplant
n) Any relapse of ALL after stem cell transplant
o) Any relapse of Burkitt's or other CD19+ and/or CD22+ lymphoma
Note patients with isolated CNS relapse meeting one or more of the criteria above were eligible for the study

Exclusion Criteria for registration were as follows.
a) Active Hepatitis B, C or HIV infection
b) Oxygen saturation s 90% on air
c) Bilirubin >3× upper limit of normal
d) Creatinine >3× upper limit of normal
e) Women who are pregnant or breastfeeding
f) Stem Cell Transplant patients only: active significant (overall Grade a 11, Seattle criteria) acute GVHD or moderate/severe chronic GVHD (NIH consensus criteria) requiring systemic steroids.
g) Inability to tolerate leukapheresis
h) Kamofsky (age a 10 years) or Lansky (age <10) score s 50%
i) Pre-existing significant neurological disorder (other than CNS involvement of underlying haematological malignancy)

The exclusion criteria for CD19/22CAR T-cell infusion were as follows.
a) Severe intercurrent infection at the time of scheduled CD19/22 CAR T-cell infusion
b) Requirement for supplementary oxygen or active pulmonary infiltrates at the time of scheduled CD19/22 CAR T-cell infusion
c) Allogeneic transplant recipients with active significant acute GVHD overall grade ≥11 or moderate/severe chronic GVHD requiring systemic steroids at the time of scheduled CD19/22 CAR T-cell infusion. Note: Such patients will be excluded until the patient is GVHD free and off steroids

The study design was a multi-center, non-randomized, open label phase I clinical trial of an Advanced Therapy Investigational Medicinal Product (ATIMP) in children and young adults with high risk, relapsed CD19+ and/or CD22+ hematological malignancies (chiefly ALL and Burkitt's lymphoma). The ATIMP tested in cohort 1 and 2 of this study was CD19CAT-41BBζ CAR T-cells (referred to as CD19CAR T-cells). The ATIMP tested in cohort 3 of this study was CD19CAT-CD22 9A8-41BBZ CAR T-cells (as described in the Examples above). A total of thirty-three patients will be treated at three participating sites. Anticipated recruitment will be over 5.5 years.

Thirteen patients were screened and enrolled, one was withdrawn due to progressive viral infection precluding lymphodepletion. Twelve patients were treated. The patients underwent an unstimulated leukapheresis which was sent to the Gene and Cell Therapy facility, Great Ormond Street Hospital (GCT-GOSH, London, UK) for manufacture of CD19/22CAR T-cells. ATIMP manufacture took about fifteen days. During this period, patients received “holding” chemotherapy to maintain disease control.

Prior to CD19/22CAR T-cell infusion patients received lymphodepleting chemotherapy: fludarabine 30 mg/m2 i.v. day −7 to −3 and cyclophosphamide 0.5 g/m2 i.v. day −4 to −2. A single dose of 10⁶/kg cryopreserved CD19/22CAR T-cells was administered intravenously. Patients were/will be followed up regularly (with daily, weekly and monthly visits) until two years post-CD19/22CAR T-cell infusion. After two years, patients will continue to be followed up annually until 10 years post ATIMP infusion.

### Results

The patient characteristics for Cohort 3 are summarized in the following Table 13, Table 14, and Table 21. The median age was 12 years (range 3.7-20.5 years). This was a heavily pre-treated cohort with a median of 3 prior lines of therapy (range 2-6). Half (6/12) had relapsed post allogeneic stem cell transplantation (SCT). 6 patients had received prior Blinatumomab, of whom 2 had also received Inotuzumab. Four patients had relapsed post Tisagenlecleucel therapy. Three had detectable CD19-negative disease at enrolment. The leukaemia was completely CD19-negative and in addition had a 5% CD22 negative population in one case. In the others, there was a significant proportion (>5%) of the disease which did not express CD19. Six patients had isolated extramedullary (EM) relapse (of which 2 were with non-CNS EM disease). By virtue of the above characteristics, all patients were ineligible for Tisagenlecleucel therapy at the time of enrolment. Bone marrow disease burden prior to lymphodepletion was >5% blasts in 4/12 patients, <5% (or positive for measurable residual disease—MRD) in 5/12 patients and MRD negative in 3/12 patients.

FIG. 13 shows the Consort diagram of Cohort 3.

TABLE 13

| Total | n = 12 | |
|---|---|---|
| Median age at registration | 12 yrs | |
| | (range = 3.7-20.5) | |
| Indication | | |
| Post SCT relapse | 6 (55%, 3 isolated | |
| | extramedullary) | |
| 1st relapse | 2 | (18%) |
| 2^{nd} relapse | 8 | (73%) |
| >2^{nd} relapse | 1 | (9%) |
| Median number of lines of | 3 | (2-6) |
| prior Rx | | |
| Prior | 6 | (55%) |
| Inotuzumab/Blinatumomab | | |
| Prior CD19 CAR T cell | 4 | (36%) |
| therapy | | |
| CD19-ve disease | 3 | (27%) |
| BM status | | |
| pre-lymphodepletion | | |
| Morphological relapse | 4 (36% + 1 NE with | |
| (>5% blasts) | 6% mol MRD) | |
| MRD 2-5% | 1 | (9%) |
| MRD 10⁻²-10⁻⁵ | 3 | (27%) |
| MRD negative | 3 | (18%) |

None eligible for Kymriah (4 previous Kymriah, 5 EM disease, 3 CD19-ve component at enrolment)

TABLE 14

| | | | | | Prior CD19 CAR |
|---|---|---|---|---|---|
| | | | | | T cell therapy |
| Patient | | CD19 | Prior | Prior | (Kymriah/ |
| No. | Cytogenetics | neg | Inotuzumab | Blinatumomab | Tisagenlecleucel) |
| CPL26 | p11.2 t(7, 9) | | Yes | Yes | Yes |
| CPL27 | cryptic t(12, 21) t (7, 12) + 10 | | | Yes | |
| CPL28 | t(9; 22) | | | | Yes |
| CPL29 | abnormal karyotype of 46 | | | | |
| | chromosomes with a | | | | |
| | translocation between 3q and | | | | |
| | one 5q and an interstitial deletion | | | | |
| | within one 12p. FISH: 67% of | | | | |
| | cells had loss of one copy of ETV | | | | |
| | 6, consistent with DCZ (12p) | | | | |
| CPL30 | high hyperdiploid karyotype, | | | | |
| | partial gain of 1q | | | | |
| CPL31 | | | | Yes | Yes |
| CPL32 | | Yes | Yes | Yes | |
| CPL33 | Low hypodiploidy | Yes | | | |
| | TP53 Pathogene variant | | | | |
| CPL35 | Hyperdiploidy | Yes | | | Yes |
| CPL36 | IAMP21 | | | Yes | |
| CPL37 | T(1, 7) TCF3-PBX1 Fusion with | | | Yes | |
| | loss of 1 allele of KMTZA | | | | |
| CPL38 | t (1, 19) TCF3 - PBXI | | | | |
| | translocation and deltq | | | | |

TABLE 21

| | | | | Lines of | Disease level | CD19/22 | |
|---|---|---|---|---|---|---|---|
| | Disease | EM disease | | treatment | by flow/mol | expression | |
| Patient | status at | at | Previous | prior to | MRD pre- | at | Comorbidities at |
| No. | enrollment | enrolment | SCT | CARPALL | lymphodepletion | enrolment | lymphodepletion |
| CPL26 | 2^{nd} relapse | no | no | 6 | 0.12% | +/+ | |
| | | | | | 1.3 × 10e−3 | | |
| CPL27 | 2^{nd} | CNS | yes | 5 | 0.39% | +/+ | |
| | relapse | | | | 1 × 10e−2 | | |
| CPL28 | ³rd | No | yes | 6 | NE/ | +/+ | leukoencephalopathy, |
| | relapse | | | | 6 × 10e−2 | | peripheral neuropathy, |
| | | | | | | | fungal infection |
| CPL29 | 2^{nd} | CNS | yes | 3 | 0.069% | +/+ | |
| | relapse | | | | 7 × 10e−4 | | |
| CPL30 | 2^{nd} | CNS | yes | 3 | Negative/ | +/+ | |
| | relapse | | | | Negative | | |
| CPL31 | 2^{nd} | no | yes | 4 | 85% | +/+ | |
| | relapse | | | | | | |
| CPL32 | 2^{nd} | adenopathy/ | no | 3 | 12% | 10% blasts | |
| | relapse | pelvic mass | | | 2.8 × 10e−1 | CD19 neg/+ | |
| CPL33 | 1^{st} | no | no | 2 | 2.3%/NE | −/+ | Li-Fraumeni |
| | relapse | | | | | | |
| CPL35 | 2^{nd} | no | no | 4 | 18.6% | 100% blasts | |
| | relapse | | | | | CD19 neg/ | |
| | | | | | | 5% blasts | |
| | | | | | | CD22 neg | |
| CPL36 | 2^{nd} | chest wall | yes | 3 | Negative/ | +/+ | |
| | relapse | | | | Negative | | |
| CPL37 | 1^{st} relapse | CNS and | no | 3 | 7.4% | +/+ | |
| | | spine | | | | | |
| CPL38 | 1^{st} relapse | Yes CNS | no | 2 | Negative/ | +/+ | |
| | | | | | Negative | | |

EM: extramedullary,
SCT: allogeneic stem cell transplant,
Mol: molecular,
MRD: minimal residual disease,
CNS: central nervous system,
NE: not evaluable.

All but one patient received the target dose of 1×10⁶/kg total CAR T cells with a median total CAR transduction efficiency of 83.2% (range 60.8-92.6%). One patient received a total of 0.9×10⁶/kg CART cells per kg ideal body weight.

The details of toxicity per patient is shown in the following Table 15.

TABLE 15

| | | | | G3-4 | | |
|---|---|---|---|---|---|---|
| Patient | | Tocilizu- | | Cytopaenia | Other G3-4 | Onset/ |
| No. | CRS | mab | ICANS | > D28 | toxicity | Resolution |
| CPL26 | G2 | | No | No | None | |
| CPL27 | G2 × | | G4 ICANS/ | G4 ↓pl | None | ICANS D39- |
| | 2 | | neurotoxicity | G4 ↓neuts | | resolved with |
| | | | (bilat | →CD34 top-up | | sequelae |
| | | | weakness + | | | CD34 top-up -> |
| | | | aphasia) | | | cytopenia |
| | | | Rx | | | resolution |
| | | | dex/anakinra | | | |
| CPL28 | No | | No | NE | None | Cytopenia related |
| | | | | | | to NR |
| CPL29 | G1 | Y | No | G4 ↓neuts | None | Cytopenia |
| | | | | | | resolved |
| CPL30 | G1 | | G2 | G3 ↓neuts | None | Cytopenia |
| | | | | | | resolved |
| CPL31 | G2 | Y | G1 | G4 ↓neuts | G4 | All resolved |
| | | | | G3 ↓pl | neutropaenic | |
| | | | | | sepsis + colitis | |
| | | | | | G4 Fibrinogen ↓ | |
| CPL32 | G2 | Y | G1 | G3 ↓neuts > D30 | G3 lung | Cytopenia |
| | | | | | infection | resolved |
| | | | | | (fungal) | |
| CPL33 | G2 | Y | No | G4 ↓pl > D30 | G3 Febrile | Cytopenia |
| | | | | G4 ↓neuts > D30 | neutropenia | resolved |
| CPL35 | G1 | | G1 | NE | G3 TLS + | |
| | | | | (died before D30) | abdominal pain | |
| CPL36 | G1 | | No | G3 ↓neuts > D30 | No | Cytopenia |
| | | | | | | resolved |
| CPL37 | G2 | Y | No (G1 tremor) | G4 ↓neuts > D30 | No | Neutropenia |
| | | | | G3↓pl > D28 | | not resolved |
| | | | | | | at last FU (2 mo) |
| CPL38 | G1 | | No | G4 Neutrophil | G3 anemia | Cytopenia |
| | | | | count decreased at | G3 hypokalemia | resolved |
| | | | | day 63 post | G3 diarrhea | |
| | | | | infusion | G3 device | |
| | | | | | related infection | |

The toxicity in n=12 patients is summarized as follows (Table 16):
11/12 patients presented CRS: Grade 1 (G1) n=5, G2 n=6, lasting a median of 5 days (range 1-19). In most cases CRS occurred early post infusion of CAR T cells (median 9, range 4-85 days), however in one case, delayed CRS developed after infusion of a CD34⁺ selected donor stem cell top-up infusion, presumed related to CAR T expansion after infusion of CD19′ B cells/precursors. No patients developed severe (≥grade 3) CRS, and no patients were admitted in paediatric intensive care unit (PICU) for CRS. Tocilizumab was given to 5 patients.
6/12 patients developed immune effector cell-associated neurotoxicity syndrome (ICANS)(G1 n=4, G2 n=1, G4 n=1). Grade 1-2 ICANS was noted at a median time from CAR-T cell infusion of 10 days (range 2 to 13) and self-resolved in all of them;
1 patient had G4 ICANS, which was associated with leukoencephalopathy which was radiologically, clinically and pathologically indistinguishable from fludarabine neurotoxicity;
Cytopenias, as for cohort 1 (treated with CD19CAT T-cells), were significant with 10/12 prolonged G3-4 cytopaenias >D28, 8/10 resolved by last follow-up (FU); 1 patient required post CD34+ selected donor stem cell boost at 2.5 months post infusion. Despite this there were only 4 episodes of grade 4 infection and no grade 5 toxicity was recorded;
The rates of grade 3-4 infection were as expected for a heavily pre-treated cohort,
Importantly, there were no grade 5 toxicities (deaths) and no evidence of hemophagocytic lymphohistiocytosis as has been noted in other CD22 CAR studies [Lichtenstein, et al. (2021) Blood 138:2469-84];
Other grade 3-4 toxicities are listed in Table 16.

TABLE 16

| | |
|---|---|
| | N = 12 |
| Criteria | N (%) |

CRS - Maximum Grade (ASTCT)

| | | |
|---|---|---|
| Grade 1 | 5 | (41%) |
| Grade 2 | 6 | (50%) |

| | |
|---|---|
| Grade 3-4 | 0 |

ICANS - Maximum Grade (ASTCT)

| Grade 1 | 4 | (36%) |
|---|---|---|
| Grade 2 | 1 | (8%) |
| Grade 3 | 0 | |
| Grade 4 (MRI leukoencephalopathy) | 1 | (8%) |

Cytopenia not resolving by/recurring after
day 28 Maximum Grade (CTCAE)

| Grade 1 | 0 | |
|---|---|---|
| Grade 2 | 1 | (8%) |
| Grade 3 | 2 | (17%) |
| Grade 4 | 8 | (67%) |

B cell aplasia

| | | |
|---|---|---|
| At day 30 | 11 | (92%) |
| At last follow up (median 8.7 months) | 7 | (58%) |

| | |
|---|---|
| Criteria: | N = 12 |
| Other, non-laboratory Grade 3-4 | N (%) |

Infection

| | | |
|---|---|---|
| Grade 3 (catheter-related, lung infection, device related) | 3 | (25%) |
| Grade 4 (sepsis, lung infection) | 2 | (17%) |

Febrile neutropenia

| | | |
|---|---|---|
| Grade 3 | 4 | (33%) |

Typhilitis

| | | |
|---|---|---|
| Grade 4 | 1 | (8%) |

Diarrhoea

| | | |
|---|---|---|
| Grade 3 | 1 | (8%) |

Respiratory

| | | |
|---|---|---|
| Grade 3 (pleural effusion) | 1 | (8%) |
| Grade 4 (hypoxia) | 1 | (8%) |

Hypotension/Hypertension

| | | |
|---|---|---|
| Grade 3 | 3 | (25%) |

Tumour Lysis syndrome

| | | |
|---|---|---|
| Grade 3 | 1 | (8%) |

Pain

| | | |
|---|---|---|
| Grade 3 (abdominal, bone) | 2 | (17%) |

Depression

| | | |
|---|---|---|
| Grade 3 | 1 | (8%) |

CAR T cell kinetics in the peripheral blood and bone marrow were measured by flow cytometry using anti-idiotype antibodies to detect CAR T cell populations bearing either or both CARs as well as qPCR for the CD19CAR and the CD22CARs. By flow cytometry, we noted rapid expansion of all CAR T cell populations peaking at 14 days post infusion. Median time to loss of both single transduced CD19 and double transduced CD19/22 CART population by flow cytometry in peripheral blood was 5 months, whilst median time to loss of CD22 single transduced CART cells was 7 months.

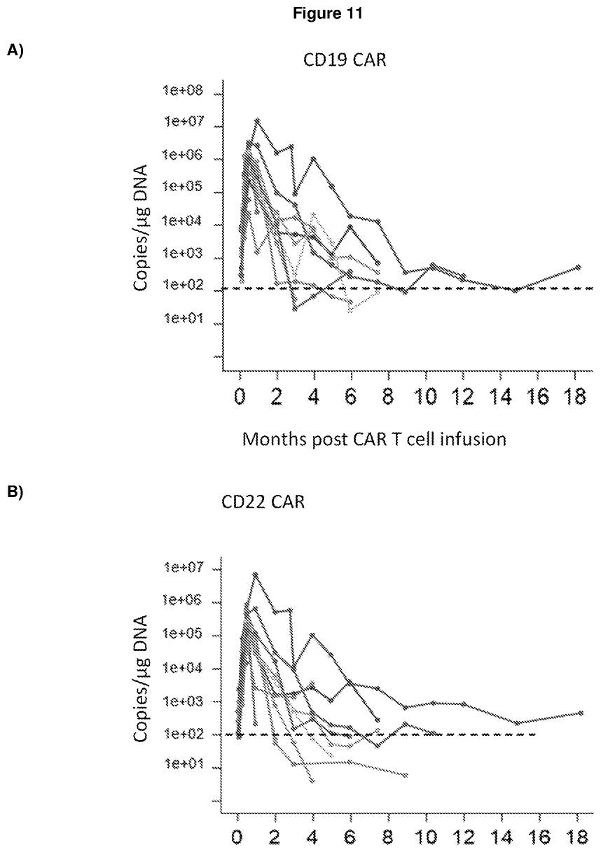

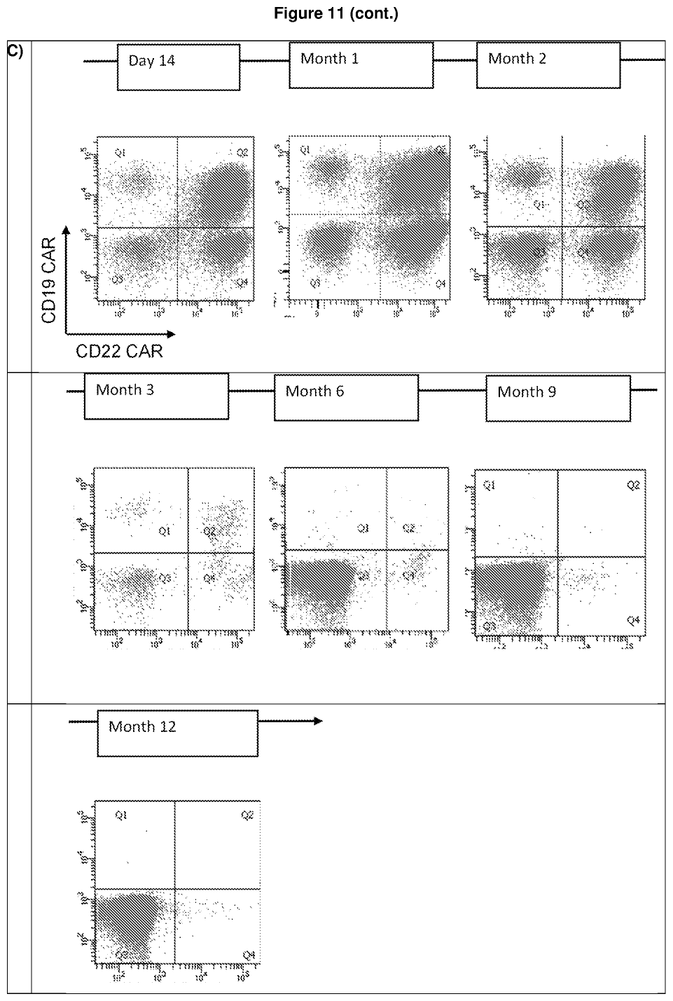

In six exemplary patients who were infused with the CD19/22CAR T-cell product, expression of both the CATi9CAR and 9A8 CAR by flow cytometry with specific anti-idiotype antibodies was detected in the blood of all patients 28 days after CD19/22CAR T-cell product administration (FIG. 6). Data from a representative patient by flow cytometry with the staining for the CD19 CAR on the y axis and for the CD22 CAR on the x axis revealed an extra-ordinary early expansion (FIG. 11C).

CAR-T cell expansion and persistence determined by qPCR for the CAR transgene is shown in FIG. 11A, 11B. The expansion and persistence kinetics were broadly matched with an early expansion to very high levels, peaking at 14 days post infusion, followed by a contraction. Persistence of CD19 and CD22 CAR-T cells generally correlated. The median duration of B cell aplasia had not been reached by the data cutoff date.

The pharmacokinetic analysis is summarized in the following Table 17. Pharmacokinetic analysis showed similar early expansion of both CARs (Cmax for CD19 CAR: 937,947.80 copies/ug DNA; Cmax for CD22 CAR: 270,171.20 copies/ug DNA; median time to Cmax being 14 days for both CARS (range 13 to 28 days) and very high cumulative CAR T cell exposure in the first 28 days (AUC0-28 CD19CAR: 9,492,498.00 copies/ug DNA; CD22CAR: 2,586,767.00 copies/ug DNA). Using qPCR, CD19 CAR T cells were detectable at last follow-up in 7/12 and CD22CAR T cells in 5/12 patients. The median half-life of CAR T cells in 10 evaluable patients was 15.4 days (range: 2.2 to 34.4) for CD19 and 17.7 days (range: 1.2 to 40.2) for CD22 (determined in 10 evaluable patients). Seven of 12 patients have ongoing B cell aplasia and the median duration of B cell aplasia for the whole cohort has not yet been reached. The pharmacokinetic data comparing the maximal concentration (Cmax) and area under the curve (AUC) in the first 28 days comparing the first cohort and the current cohort. The peak concentration was a log higher for the CD19CAR component than for cohort 1 when comparing geometric means, and higher than for the CD22 CAR by about 4 fold. The AUC exposure within the first 28 days was also approximately a log higher for CD19 compared to cohort 1 and about 5-fold that for the CD22 CAR.

TABLE 17

| | | CARPALL3 | |
|---|---|---|---|
| | CARPALL 1 | (n = 12) | |
| | (n = 14) | CD19 | CD22 |
| Time of last | | | |
| measurement (days) | | | |
| Median | 267 | 202.5 | 202.5 |
| Mean | | 212.5 | 212.5 |
| Range | 28 to 728 | 14 to 553 | 14 to 553 |
| Cmax concentration | | | |
| (copies/ug DNA) | | | |
| Geometric mean | 128,912 | 937,947.80 | 270,171.20 |
| Mean | | 2,596,620 | 902,562.30 |
| CV % | 330.2 | 528.08 | 356.59 |
| Time to Cmax | | | |
| Median | 14 | 14 | 14 |
| Mean | | 16.2 | 17.3 |
| Range | 7 to 14 | 13 to 28 | 13 to 28 |
| AUC (day 0 to day 28) | | | |
| (copies/ug DNA) | | | |
| Geometric mean | 1,721,355 | 9,492,498 | 2,586,767 |
| Mean | | 25,300,000 | 8,017,348.00 |
| CV % | 506.2 | 528.08 | 356.59 |
| N patients with | 11 | 7 | 5 |
| CART >=100 copies/ug | | | |
| at last follow-up | | | |
| Median follow-up | 14.4 | 8.7 | 8.7 |
| in months | | | |

Cohort 3 patient response is summarized in the following Table 18. At one month post CAR T cell infusion, 10/12 (83%) patients were in a complete remission with or without haematological recovery (CR/CRi). Three of these patients were in continuing CR/CRi having attained this status prior to lymphodepletion and CAR T infusion. 9 of these 10 patients with CR/CRi had no MRD detectable by flow cytometry or PCR. One patient with MRD below the quantitative range at month 1 had cleared this by month 2 post infusion, resulting in an MRD negative CR rate of 100% amongst responders at this time-point. Importantly, of 3 patients with CD19-negative disease at screening, 2 attained an MRD negative CR/CRi supporting the therapeutic efficacy of CAR T cells bearing the CD22CAR in our product. Two patients had no response, one with CD19+/CD22+ disease and one with progression of double CD19−/CD22− disease which had been present as a minor (5%) population prior to CAR T cell infusion. Both patients subsequently died of disease, at 3 months and 15 days post infusion respectively.

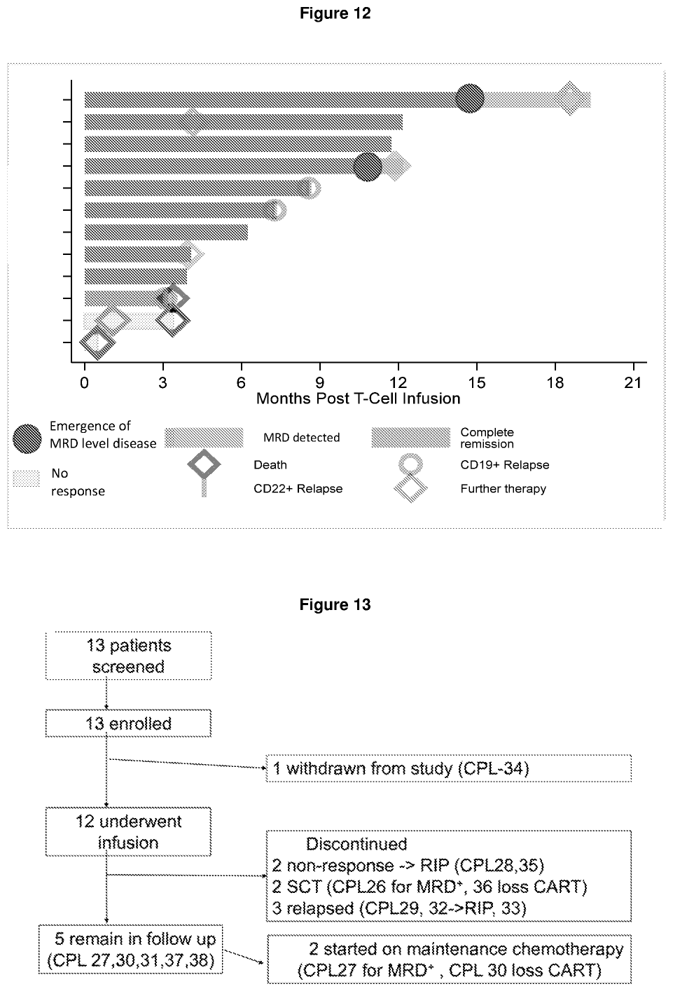

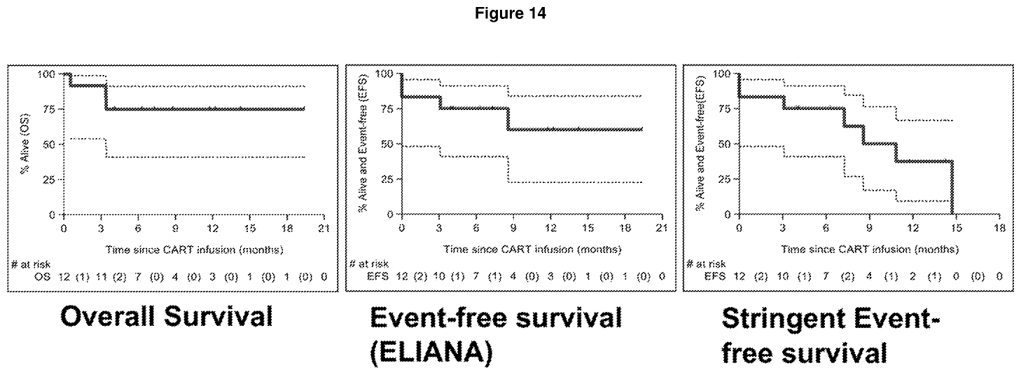

Among the 10 patients who attained an MRD negative CR/CRi, 3 have subsequently relapsed at 3, 7.5 and 9 months post infusion with CD19⁺/CD22⁺ disease, in 2/3 cases associated with early loss of CAR T cell persistence before 6 months post infusion. In a further 2 cases, there was emergence of MRD level disease (CD19*CD22* in both cases) for which the patients received further therapy including SCT in one case and maintenance chemotherapy in a second case. They both are in disease remission. Due to early loss of CAR T cell persistence at 3 and 4 months post infusion, 2 patients went on to have further therapy whilst in MRD negative remission. This included allo-SCT in one case and maintenance chemotherapy in the other (FIG. 12, Table 22). At a median follow-up of 8.7 months (95% CI: 3.9 to 12.2), 5 out of 10 responding patients are alive and have remained disease-free and there have been no relapses due to antigen loss to date. Overall survival was 75% (95% CI: 41-91%) at 6 and 12 months (FIG. 14). Using the same EFS definition as in the ELIANA study, 6 and 12 month EFS were 75% (95% CI: 41-91%) and 60% (95% CI: 17-84%) respectively (FIG. 14). We also applied a more stringent event definition which included events as above but importantly included need for further therapy either for MRD emergence or early loss of CAR T cells. The 6 and 12 month stringent EFS were 75% (95% CI: 41-91%) and 38% (95% CI: 9-67%) respectively (FIG. 14). The median duration of remission in responding patients was 9.9 months.

TABLE 18

| | | CAR T | B cell | | | |
|---|---|---|---|---|---|---|
| | Response | detectable | aplasia at | | | |
| | D28 | at last FU | last FU | Current status | FU | |
| CPL26 | MRD −ve CR | No | Yes | CD19/22 +ve relapse (10.5 m) | 12 | m |
| CPL27 | MRD −ve CR | No | Yes | Flow MRD −ve CR (12 m) | 19 | m |
| | | | | Neurol improving | | |
| CPL28 | NR. BM | Yes | Yes | Non-responder: died disease | 3 | m |
| | MRD +ve | | | progression | | |
| | CNS relapse | | | | | |
| CPL29 | MRD −ve CR | Yes (CD19 | Yes | CD19+CD22+ isol CNS rel (9 m) | 9 | m |
| | | and CD22 | | | | |
| | | present | | | | |
| | | in bone | | | | |
| | | marrow) | | | | |
| CPL30 | MRD −ve CR | No | No | MRD −ve CR on maintenance for | 14.2 | m |
| | | | | early loss CART @ 4 mo (9 m) | | |
| CPL31 | MRD −ve CR | No | No | MRD −ve CR (7.5 m) | 12 | m |
| CPL32 | CR | Yes (bone | No | CD19+CD22+ BM + EM relapse | 3 | m |
| | (MRD POQR) | marrow) | | (3 m). | | |
| | | | | Died disease progression | | |
| CPL33 | Flow MRD −ve | Yes | Yes | Flow MRD −ve CR (5 mo) | 7 | m |
| | CR (no mol | | | | | |
| | marker) | | | | | |
| CPL35 | NR relapsed | Yes | N/A | Non-responder: died | 0.5 | m |
| | D13 | | | CD19-CD22-disease | | |
| | | | | progression | | |
| CPL36 | MRD −ve CR | No | No | MRD −ve CR (4 mo) | 4 | m |
| | with clearance | | | | | |
| | EM disease | | | | | |
| CPL37 | MRD −ve | Yes | Yes | MRD −ve CR (2 mo) | 6 | m |
| CPL38 | Molecular | Yes | No | | 4 | m |
| | remission | | | | | |

A summary of the patient outcomes is shown in the Swimmer's plot of FIG. 12, survival curves of FIG. 14, Table 19, which amalgamates the patient characteristics and outcome data, and Table 22, which shows the summary of response and relapses. At a median follow-up of 8.7 months, there have been no cases of antigen negative relapse and 5/10 responding patients in MRD negative complete response (CR).

The 12 month event-free survival (EFS), as defined in the ELIANA study was 60% and aligned with that seen in the pivotal study for tisagenlecleucel (Kymriah), despite a third of the cohort here having already failed that therapy. A stringent EFS in which the event definition included MRD emergence or further therapy was also documented. Twelve-month stringent EFS was 38% and, again, this aligned to UK real world data on stringent EFS following tisagenlecleucel (Kymdiah™) therapy.

TABLE 19

| | | | “Stringent” |
|---|---|---|---|
| | Overall | Event-Free | Event-Free |
| | Survival | Survival | Survival |
| | N = 12 | N = 12 | N = 12 |
| N. of events | 3 (25%) | 4 (33%) | 7 (58%) |
| 12 month rate | 75% (41% | 60% (23% | 38% (9% |
| (95% CI) | to 91%) | to 84%) | to 67%) |

TABLE 22

| Total | N = 12 | |
|---|---|---|
| Molecular MRD neg CR/CRi by d60 | 10 | (83%) |
| Disease progression | 2 | (17%) |

Relapse

| Ag negative relapse | 0 | |
|---|---|---|
| CD19+/CD22+ relapse | 5 | (50%) |

The interim conclusions of this study are as follows:
AUTO1/22 ATIMP: it was possible to reproducibly generate a product that was balanced in CD19CAR vs CD22CAR, with predominantly central memory phenotype;
Safety profile was favourable: no severe CRS, 1 patient with G4 ICANS but was atypical and clinically resembled fludarabine neurotoxicity due to presence of leukoencephalopathy;
Excellent CAR-T cell expansion, including of CD22 CAR population.
Median CAR-T cell persistence in bone marrow by flow was 6 months for CD19 and CD19/22.
Poor risk patient cohort: no patient was eligible for Kymriah (4 patients had failed Kymriah); 3 patients had CD19 negative disease; 3 patients had non-CNS extramedullary disease.
10/12 demonstrated complete response (MRD negative CR/CRVCCR), despite poor risk nature of patient cohort, with 2 non-responders.
2/3 patients with prior CD19 negative disease achieved CR, demonstrating efficacy of CD22CAR.
despite poor risk nature of patient cohort (4 patients had failed Kymriah, 3 patients had CD19 negative disease), 1 year EFS was 60% and equivalent to that of the ELIANA study (Tisagenlecleucel/Kymriah), and “stringent EFS” of 38% similar to UK real world data with Kymriah.
3 patients had CD19+CD22+ relapses, and 2 patients had CD19+CD22+ MRD emergence. All were associated with non-persistence.
Antigen positive relapse was associated with loss of CAR-T cells, a major cause of treatment failure.
No antigen negative relapse seen in responding patients, suggesting effective dual targeting.
At median follow-up of 8.7 mo, 5/10 responding patients in MRD negative had CR (4-12 months), 2 after further therapy for early loss of CAR T-cell persistence.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of treating high risk/relapsed CD19+ or CD22+ haematological malignancy in a patient comprising administering autologous CD19/22 CAR T-cells to the patient.

2. Autologous CD19/22 CAR T-cells for use in the treatment of high risk/relapsed CD19+ or CD22+ haematological malignancy.

3. (canceled)

4. The method of claim 1, wherein the age of the patient is twenty-four years or younger.

5. The method of claim 1, wherein the haematological malignancy is acute lymphoblastic leukemia (ALL), or a CD19+ or CD22+ lymphoma.

6. The method of claim 5, wherein the lymphoma is Burkitt's lymphoma.

7. The method of claim 1, wherein the patient has:
a) resistant disease (>5% blasts) at end of UKALL 2019 guidelines or equivalent induction,
b) ALL with persisting high level MRD at 2nd time point of frontline national protocol (currently MRD>10⁻⁴ at week 14 UKALL2019 guidelines or equivalent),
c) high risk infant ALL (age <6 months at diagnosis with MLL gene rearrangement and either presenting white cell count >300×10⁹/L or poor steroid early response (circulating blast count >1×10⁹/L following 7 day steroid pre-phase of induction as per national guidelines or equivalent),
d) intermediate risk infant ALL with MRD>10⁻³ at end of induction following national guidelines or equivalent),
e) high risk first relapse (as defined by updated IntreALL 2019 classification: bone marrow or combined relapse within thirty months of diagnosis),
f) standard risk relapse in patients with high risk cytogenetics (defined as BCR-ABL, KMT2A rearrangement, near-haploidy (<30 chromosomes) and low hypodiploidy (30-39 chromosomes), iAMP21 and TCF3-HLF translocations),
g) standard risk relapse with bone marrow minimal residual disease (MRD) >10⁻³ at end of re-induction,
h) any refractory relapse of ALL (defined as >1% blasts by flow cytometry after at least one cycle of standard chemotherapy), or
i) any relapse of CD22+ lymphoma.

8. The method of claim 7, wherein the patient has an isolated CNS relapse meeting one or more of a)-i).

9. The method of claim 1, wherein the patient has received one or more lines of prior therapy.

10. The method of claim 9, wherein the patient has received one or more of inotuzumab ozogamicin, blinatumomab and tisagenlecleucel.

11. The method of claim 1, wherein the patient is administered a single dose of 0.5×10⁶ CAR T-cells/kg, 0.75×10⁶ CAR T-cells/kg, 1×10⁶ CAR T-cells/kg, or 1.2×10⁶ CAR T-cells/kg.

12. The method of claim 11, wherein the administration is an intravenous injection, preferably through a Hickman line or peripherally inserted central catheter.

13. The method of claim 1, wherein the CD19/22 CAR T-cell product expresses a chimeric antigen receptor (CAR) comprising a CD19-binding domain which comprises;
a) a heavy chain variable region (VH) having
complementarity determining regions (CDRs) with
the following sequences:
(SEQ ID NO: 1)
CDR1 - GYAFSSS;
(SEQ ID NO: 2)
CDR2 - YPGDED
(SEQ ID NO: 3)
CDR3 - SLLYGDYLDY;
and
b) a light chain variable region (VL) having CDRs
with the following sequences:
(SEQ ID NO: 4)
CDR1 - SASSSVSYMH;
(SEQ ID NO: 5)
CDR2 - DTSKLAS
(SEQ ID NO: 6)
CDR3 - QQWNINPLT.

14. The method of claim 13, wherein the CD19-binding domain comprises a VH domain having the sequence shown as SEQ ID NO: 7 and/or or a VL domain having the sequence shown as SEQ ID NO: 8 or a variant thereof having at least 95% sequence identity.

15. The method of claim 14, wherein the CD19-binding domain comprises an scFv in the orientation VH-VL.

16. The method of claim 15, wherein the CD19-binding domain comprises the sequence shown as SEQ ID NO: 9 or a variant thereof having at least 90% sequence identity.

17-21. (canceled)

22. The method of claim 1, wherein the CD19/22 CAR T-cell product expresses a chimeric antigen receptor (CAR) comprising a CD22-binding domain which comprises
a) a heavy chain variable region (VH) having CDRs
with the following sequences:
(SEQ ID NO: 58)
CDR1 - NFAMA;
(SEQ ID NO: 59)
CDR2 - SISTGGGNTYYRDSVKG
(SEQ ID NO: 60)
CDR3 - QRNYYDGSYDYEGYTMDA;
and
b) a light chain variable region (VL) having CDRs
with the following sequences:
(SEQ ID NO: 61)
CDR1 - RSSQDIGNYLT;
(SEQ ID NO: 62)
CDR2 - GAIKLED
(SEQ ID NO: 63)
CDR3 - LQSIQYP.

23. The method of claim 22, wherein the CD22-binding domain comprises a VH domain having the sequence shown as SEQ ID NO: 64 and/or or a VL domain having the sequence shown as SEQ ID NO: 65 or a variant thereof having at least 95% sequence identity.

24. The method of claim 23, wherein the CD22-binding domain comprises an scFv in the orientation VH-VL.

25. The method of claim 24, wherein the CD22-binding scFv comprises the sequence shown as SEQ ID NO: 66 or a variant thereof having at least 90% sequence identity.

26-30. (canceled)

31. The method of claim 1, wherein the autologous CD19/22 CAR T-cells comprise a CD19/22 CAR T-cell product comprising CAT19CAR and 9A8CAR CARs.


SEQUENCE LISTING (91 sequences)

SEQ ID NO: 1 - protein, 7 aa, synthetic construct
  REGION 1..7: note = Synthetic
GYAFSSS

SEQ ID NO: 2 - protein, 6 aa, synthetic construct
  REGION 1..6: note = Synthetic
YPGDED

SEQ ID NO: 3 - protein, 10 aa, synthetic construct
  REGION 1..10: note = Synthetic
SLLYGDYLDY

SEQ ID NO: 4 - protein, 10 aa, synthetic construct
  REGION 1..10: note = Synthetic
SASSSVSYMH

SEQ ID NO: 5 - protein, 7 aa, synthetic construct
  REGION 1..7: note = Synthetic
DTSKLAS

SEQ ID NO: 6 - protein, 9 aa, synthetic construct
  REGION 1..9: note = Synthetic
QQWNINPLT

SEQ ID NO: 7 - protein, 119 aa, Mus sp.
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSS

SEQ ID NO: 8 - protein, 107 aa, Mus sp.
QIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 9 - protein, 241 aa, Mus sp.
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 10 - protein, 488 aa, synthetic construct
  REGION 1..488: note = Synthetic
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 483 aa, synthetic construct
  REGION 1..483: note = Synthetic
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 487 aa, synthetic construct
  REGION 1..487: note = Synthetic
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 527 aa, synthetic construct
  REGION 1..527: note = Synthetic
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPTTTPAPRPPTPAPTIASQPLSLRPEACRPAAGGAVHTRGLDFACDIFWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 465 aa, synthetic construct
  REGION 1..465: note = Synthetic
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPAEPKSPDKTHTCPPCPKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 681 aa, Homo sapiens
MGTSLLCWMALCLLGADHADAQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 5 aa, synthetic construct
  REGION 1..5: note = Synthetic
SYWMN

SEQ ID NO: 17 - protein, 16 aa, synthetic construct
  REGION 1..16: note = Synthetic
QIWPGDGDTNYNGKFK

SEQ ID NO: 18 - protein, 15 aa, synthetic construct
  REGION 1..15: note = Synthetic
RETTTVGRYYYAMDY

SEQ ID NO: 19 - protein, 15 aa, synthetic construct
  REGION 1..15: note = Synthetic
KASQSVDYDGDSYLN

SEQ ID NO: 20 - protein, 7 aa, synthetic construct
  REGION 1..7: note = Synthetic
DASNLVS

SEQ ID NO: 21 - protein, 9 aa, synthetic construct
  REGION 1..9: note = Synthetic
QQSTEDPWT

SEQ ID NO: 22 - protein, 235 aa, Mus sp.
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 23 - protein, 236 aa, Homo sapiens
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSSDIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKR

SEQ ID NO: 24 - protein, 236 aa, Homo sapiens
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSSDIQLTQSPDSLAVSLGERATINCKASQSVDYDGDSYLNWYQQKPGQPPKVLIYDASNLVSGVPDRFSGSGSGTDFTLTISSLQAADVAVYYCQQSTEDPWTFGQGTKVEIKR

SEQ ID NO: 25 - protein, 124 aa, Mus sp.
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 26 - protein, 124 aa, Homo sapiens
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSS

SEQ ID NO: 27 - protein, 111 aa, Mus sp.
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 28 - protein, 112 aa, Homo sapiens
DIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKR

SEQ ID NO: 29 - protein, 112 aa, Homo sapiens
DIQLTQSPDSLAVSLGERATINCKASQSVDYDGDSYLNWYQQKPGQPPKVLIYDASNLVSGVPDRFSGSGSGTDFTLTISSLQAADVAVYYCQQSTEDPWTFGQGTKVEIKR

SEQ ID NO: 30 - protein, 119 aa, synthetic construct
  REGION 1..119: note = Synthetic
EVQLVESGGGLVQPKGSLKLSCAASGFTFNTYAMHWVRQAPGKGLEWVARIRSKSSNYATYYADSVKDRFTISRDDSQSMLYLQMNNLKTEDTAMYYCVVDYLYAMDYWGQGTSVTVSS

SEQ ID NO: 31 - protein, 107 aa, synthetic construct
  REGION 1..107: note = Synthetic
DIVMTQSQKFMSTSVGDRVSITCKASQNVRTAVAWYQQKPGQSPKALIYLASNRHTGVPDRFTGSGSGTDFTLTISNVQSEDLADYFCLQHWNYPFTFGSGTKLEIK

SEQ ID NO: 32 - protein, 120 aa, synthetic construct
  REGION 1..120: note = Synthetic
QVQLQQSGAELVRPGASVTLSCKASGYTFTDYEMHWVKQTPVHGLEWIGAIDPETGATAYNQKFKGKAILTADKSSSTAYMDLRSLTSEDSAVYYCTRYDYGSSPWFAYWGQGTLVTVSA

SEQ ID NO: 33 - protein, 109 aa, synthetic construct
  REGION 1..109: note = Synthetic
QAVVTQESALTTSPGETVTLTCRSSAGAVTTSNYANWVQEKPDHLFTGLIGGTNNRAPGVPARFSGSLIGDKAALTITGAQTEDEAIYFCALWNSNHWVFGGGTKLTVL

SEQ ID NO: 34 - protein, 119 aa, synthetic construct
  REGION 1..119: note = Synthetic
QVQLQQPGAELVMPGASVKLSCKASGYTFTSYWMHWVKQRPGQGLEWIGEIDPSDSYTNYNQKFKGKATLTVDKSSSTAYMQLSSLTSEDSAVYYCARGYYGSSSFDYWGQGTTLTVSS

SEQ ID NO: 35 - protein, 111 aa, synthetic construct
  REGION 1..111: note = Synthetic
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYTFGGGTKLEIK

SEQ ID NO: 36 - protein, 120 aa, synthetic construct
  REGION 1..120: note = Synthetic
QVQLKESGPGLVAPSQSLSITCTVSGFSLTSYGVHWVRQPPGKGLEWLVVIWSDGSTTYNSALKSRLSISKDNSKSQVFLKMNSLQTDDTAMYYCARHADDYGFAWFAYWGQGTLVTVSA

SEQ ID NO: 37 - protein, 107 aa, synthetic construct
  REGION 1..107: note = Synthetic
DIQMTQSPASLSASVGETVTITCRASENIYSYLAWYQQKQGKSPQLLVYNAKTLAEGVPSRFSGSGSGTQFSLKINSLQPEDFGSYYCQHHYGTPPTFGGGTKLEIK

SEQ ID NO: 38 - protein, 121 aa, synthetic construct
  REGION 1..121: note = Synthetic
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARSSTTVVDWYFDVWGTGTTVTVSS

SEQ ID NO: 39 - protein, 107 aa, synthetic construct
  REGION 1..107: note = Synthetic
DIQMTQSPSSLSASLGERVSLTCRASQEISGYLSWLQQKPDGTIKRLIYAASTLDSGVPKRFSGSRSGSDYSLTISSLESEDFADYYCLQYASYPFTFGSGTKLEIK

SEQ ID NO: 40 - protein, 118 aa, synthetic construct
  REGION 1..118: note = Synthetic
QVQVQQPGAELVRPGTSVKLSCKASGYTFTRYWMYWVKQRPGQGLEWIGVIDPSDNFTYYNQKFKGKATLTVDTSSSTAYMQLSSLTSEDSAVYYCARGYGSSYVGYWGQGTTLTVSS

SEQ ID NO: 41 - protein, 113 aa, synthetic construct
  REGION 1..113: note = Synthetic
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPWTFGGGTKLEIK

SEQ ID NO: 42 - protein, 124 aa, synthetic construct
  REGION 1..124: note = Synthetic
QVTLKESGPGILQSSQTLSLTCSFSGFSLSTSDMGVSWIRQPSGKGLEWLAHIYWDDDKRYNPSLKSRLTISKDASRNQVFLKIATVDTADTATYYCARSPWIYYGHYWCFDVWGTGTTVTVSS

SEQ ID NO: 43 - protein, 107 aa, synthetic construct
  REGION 1..107: note = Synthetic
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIK

SEQ ID NO: 44 - protein, 123 aa, synthetic construct
  REGION 1..123: note = Synthetic
QVQLQQSGAELVKPGASVKLSCKASGYTFTEYTIHWVKQRSGQGLEWIGWFYPGSGSIKYNEKFKDKATLTADKSSSTVYMELSRLTSEDSAVYFCARHGDGYYLPPYYFDYWGQGTTLTVSS

SEQ ID NO: 45 - protein, 106 aa, synthetic construct
  REGION 1..106: note = Synthetic
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELK

SEQ ID NO: 46 - protein, 121 aa, synthetic construct
  REGION 1..121: note = Synthetic
QVQLQQSGAELARPGASVKLSCKASGYIFTSYGISWVKQRTGQGLEWIGEIYPRSGNTYYNEKFKGKATLTADKSSSTAYMELRSLTSEDSAVYFCARPIYYGSREGFDYWGQGTTLTVSS

SEQ ID NO: 47 - protein, 107 aa, synthetic construct
  REGION 1..107: note = Synthetic
DIVLTQSPATLSVTPGDSVSLSCRASQSISTNLHWYQQKSHASPRLLIKYASQSVSGIPSRFSGSGSGTDFTLSINSVETEDFGIFFCQQSYSWPYTFGGGTKLEIK

SEQ ID NO: 48 - protein, 120 aa, synthetic construct
  REGION 1..120: note = Synthetic
QVQLQQPGAELVMPGASVKLSCKASGYTFTSYWMHWVKQRPGQGLEWIGEIDPSDSYTNYNQKFKGKSTLTVDKSSSTAYIQLSSLTSEDSAVYYCARWASYRGYAMDYWGQGTSVTVSS

SEQ ID NO: 49 - protein, 112 aa, synthetic construct
  REGION 1..112: note = Synthetic
DVLMTQTPLSLPVSLGDQASISCRSSQSIVHSNGNTYLEWYLQKPGQSPKLLIYKVSNRFSGVPDRFSGSESGTDFTLKISRVEAEDLGVYYCFQGSHVPWTFGGGTKLEIK

SEQ ID NO: 50 - protein, 119 aa, synthetic construct
  REGION 1..119: note = Synthetic
EFQLQQSGPELVKPGASVKISCKASGYSFTDYNMNWVKQSNGKSLEWIGVINPNYGTTSYNQRFKGKATLTVDQSSSTAYMQLNSLTSEDSAVYYCARSGLRYWYFDVWGTGTTVTVSS

SEQ ID NO: 51 - protein, 107 aa, synthetic construct; identical to SEQ ID NO: 39
  REGION 1..107: note = Synthetic

SEQ ID NO: 52 - protein, 120 aa, synthetic construct
  REGION 1..120: note = Synthetic
EVQLVESGAEVKKPGSSVKVSCKASGYTFTNYWINWVRQAPGQGLEWMGNIYPSDSFTNYNQKFKDRVTITADKSTSTVYLELRNLRSDDTAVYYCTRDTQERSWYFDVWGQGTLVTVSS

SEQ ID NO: 53 - protein, 113 aa, synthetic construct
  REGION 1..113: note = Synthetic
DIVMTQSPATLSVSPGERATLSCRSSQSLVHSNGNTYLHWYQQKPGQAPRLLIYKVSNRFSGVPARFSGSGSGAEFTLTISSLQSEDFAVYYCSQSTHVPWTFGQGTRLEIKR

SEQ ID NO: 54 - protein, 121 aa, synthetic construct
  REGION 1..121: note = Synthetic
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 55 - protein, 113 aa, synthetic construct
  REGION 1..113: note = Synthetic
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKR

SEQ ID NO: 56 - protein, 124 aa, synthetic construct
  REGION 1..124: note = Synthetic
QVTLKESGPGILQPSQTLSLTCTFSGFSLSTSGMGVGWIRQPSGKGLEWLTNIWWDDDKNYNPSLKNRLTISKDTSINQAFLKITNVDTADTATYYCARIAHYFDGYYYVMDVWGQGTSVTVSS

SEQ ID NO: 57 - protein, 107 aa, synthetic construct
  REGION 1..107: note = Synthetic
DIQMTQSPASLSASLGETVSIECLASGGISNDLAWYQQKSGKSPQLLIYAASRLQDGVPSRFSGSGSGTRYSLKISGMQSEDEADYFCQQSYKYPYTFGGGTKLELK

SEQ ID NO: 58 - protein, 5 aa, synthetic construct
  REGION 1..5: note = Synthetic
NFAMA

SEQ ID NO: 59 - protein, 17 aa, synthetic construct
  REGION 1..17: note = Synthetic
SISTGGGNTYYRDSVKG

SEQ ID NO: 60 - protein, 18 aa, synthetic construct
  REGION 1..18: note = Synthetic
QRNYYDGSYDYEGYTMDA

SEQ ID NO: 61 - protein, 11 aa, synthetic construct
  REGION 1..11: note = Synthetic
RSSQDIGNYLT

SEQ ID NO: 62 - protein, 7 aa, synthetic construct
  REGION 1..7: note = Synthetic
GAIKLED

SEQ ID NO: 63 - protein, 7 aa, synthetic construct
  REGION 1..7: note = Synthetic
LQSIQYP

SEQ ID NO: 64 - protein, 127 aa, synthetic construct
  REGION 1..127: note = Synthetic
EVQLVESGGGLVQPGRSLKLSCAASGFTFSNFAMAWVRQPPTKGLEWVASISTGGGNTYYRDSVKGRFTISRDDAKNTQYLQMDSLRSEDTATYYCARQRNYYDGSYDYEGYTMDAWGQGTSVTVSS

SEQ ID NO: 65 - protein, 107 aa, synthetic construct
  REGION 1..107: note = Synthetic
DIQMTQSPSSLSASLGDRVTITCRSSQDIGNYLTWFQQKVGRSPRRMIYGAIKLEDGVPSRFSGSRSGSDYSLTISSLESEDVADYQCLQSIQYPFTFGSGTKLEIK

SEQ ID NO: 66 - protein, 250 aa, synthetic construct
  REGION 1..250: note = Synthetic
DIQMTQSPSSLSASLGDRVTITCRSSQDIGNYLTWFQQKVGRSPRRMIYGAIKLEDGVPSRFSGSRSGSDYSLTISSLESEDVADYQCLQSIQYPFTFGSGTKLEIKRSGGGGSGGGGSGGGGSEVQLVESGGGLVQPGRSLKLSCAASGFTFSNFAMAWVRQPPTKGLEWVASISTGGGNTYYRDSVKGRFTISRDDAKNTQYLQMDSLRSEDTATYYCARQRNYYDGSYDYEGYTMDAWGQGTSVTVS

SEQ ID NO: 67 - protein, 21 aa, synthetic construct
  REGION 1..21: note = Synthetic
MGTSLLCWMALCLLGADHADA

SEQ ID NO: 68 - protein, 20 aa, synthetic construct
  REGION 1..20: note = Synthetic
METDTLLLWVLLLLVPGSTG

SEQ ID NO: 69 - protein, 21 aa, synthetic construct
  REGION 1..21: note = Synthetic
MSLPVTALLLPLALLLHAARP

SEQ ID NO: 70 - protein, 20 aa, synthetic construct
  REGION 1..20: note = Synthetic
MAVPTQVLGLLLLWLTDARC

SEQ ID NO: 71 - protein, 234 aa, Homo sapiens
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 72 - protein, 45 aa, Homo sapiens
TTTPAPRPPTPAPTIASQPLSLRPEACRPAAGGAVHTRGLDFACD

SEQ ID NO: 73 - protein, 20 aa, Homo sapiens
AEPKSPDKTHTCPPCPKDPK

SEQ ID NO: 74 - protein, 237 aa, synthetic construct
  REGION 1..237: note = Synthetic
AEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 75 - protein, 236 aa, synthetic construct
  REGION 1..236: note = Synthetic
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 76 - protein, 185 aa, synthetic construct
  REGION 1..185: note = Synthetic
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 77 - protein, 259 aa, synthetic construct
  REGION 1..259: note = Synthetic
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 78 - protein, 24 aa, Homo sapiens
IIAIAVVGALLLVALIFGTASYLI

SEQ ID NO: 79 - protein, 24 aa, synthetic construct
  REGION 1..24: note = Synthetic
IYIWAPLAGTCGVLLLSLVITLYC

SEQ ID NO: 80 - protein, 114 aa, synthetic construct
  REGION 1..114: note = Synthetic
RSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 42 aa, synthetic construct
  REGION 1..42: note = Synthetic
KRGRKKLLYIFKQPFMRPVQTTQEEDGCSCRFPEEEEGGCEL

SEQ ID NO: 82 - protein, 37 aa, synthetic construct
  REGION 1..37: note = Synthetic
RRDQRLPPDAHKPPGGGSFRTPIQEEQADAHSTLAKI

SEQ ID NO: 83 - protein, 37 aa, synthetic construct
  REGION 1..37: note = Synthetic
KRSRLLHSDYMNMTPRRPGPTRKHYQPYAPPRDFAAY

SEQ ID NO: 84 - protein, 154 aa, synthetic construct
  REGION 1..154: note = Synthetic
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 149 aa, synthetic construct
  REGION 1..149: note = Synthetic
RRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 151 aa, synthetic construct
  REGION 1..151: note = Synthetic
KRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 187 aa, synthetic construct
  REGION 1..187: note = Synthetic
KRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - DNA, 1467 bp, synthetic construct
  misc_feature 1..1467: note = Synthetic
  source 1..1467: mol_type = other DNA
atgggcaccagcctgctgtgctggatggccctgtgcctgctgggagccgaccacgccgacgcccaggtgcagctgcagcagagcggacccgagctggtgaagcccggagccagcgtgaagatcagctgcaaggccagcggctacgccttcagcagcagctggatgaactgggtgaagcagcggcctggcaagggcctggagtggatcggccggatctaccccggcgacgaggacaccaactacagcggcaagttcaaggacaaggccaccctgaccgccgacaagagcagcaccaccgcctacatgcagctgagcagcctgaccagcgaggacagcgccgtgtacttctgcgcccggagcctgctgtacggcgactacctggactactggggccagggcaccaccctgaccgtgagctctggcggtggcggctctggcggaggtggctctggcggaggcggcagccagatcgtgctgacccagagccctgccatcatgagcgccagccctggcgagaaggtgaccatgacctgcagcgccagcagcagcgtgagctacatgcactggtaccagcagaagagcggcaccagccctaagcggtggatctacgacaccagcaagctggccagcggcgtgcccgaccgcttcagcggcagcggcagcggcaccagctacttcctgaccatcaacaacatggaggccgaggacgccgccacctactactgccagcagtggaacatcaatcctctgaccttcggcgccggcaccaagctggagctgaagcggtcggatcccaccaccaccccagccccacggccacctacccctgccccaaccatcgccagccagcccctgagcctgcggcctgaagcctgcaggcctgccgccggaggagccgtgcacacaaggggcctggacttcgcctgcgacatctatatctgggcccccctggccgggacatgcggggtgctgctgctgtccctggtgattacactgtattgcaaacggggccggaagaagctgctgtacatcttcaagcagcccttcatgcggcccgtgcagaccacccaggaggaggacggctgcagctgccggttccccgaggaagaggaaggcggctgcgagctgcgggtgaagttcagccggagcgccgacgccccagcctaccagcagggccagaaccagctgtacaacgagctgaacctgggacggcgggaggagtacgacgtgctggacaagcggcggggacgggaccccgagatgggcggcaagcctcgccggaagaatccccaggagggcctgtacaacgagctgcagaaggacaagatggccgaggcctacagcgagatcggcatgaagggcgagcggcgccggggcaagggccacgacggcctgtaccagggcctgagcaccgccaccaaggacacctacgacgccctgcacatgcaggccctgccaccccggtga

SEQ ID NO: 89 - protein, 488 aa, synthetic construct; identical to SEQ ID NO: 10
  REGION 1..488: note = Synthetic

SEQ ID NO: 90 - DNA, 1491 bp, synthetic construct
  misc_feature 1..1491: note = Synthetic
  source 1..1491: mol_type = other DNA
atggagactgacaccctgctgctgtgggtgctcctgctgctggtccccggcagcaccggcgacatccagatgacgcagtccccgtccagcctgtccgccagcctcggcgaccgggtcaccatcacgtgccggtccagccaggacatcggcaactacctgacctggttccagcagaaagtcggcaggtcccctcggcggatgatctacggcgccatcaagctcgaggacggggtgcccagccggttctccggcagccgcagcggcagcgactacagcctcaccatcagcagcctcgagtccgaggacgtggccgactaccagtgcctgcagagcattcagtaccccttcaccttcggctccgggacgaagctcgagatcaagcggagcggagggggcgggtccggaggcggcgggtccggaggcggcgggagcgaggtgcagctggtcgagtccggtggcggcctggtgcagcctggacggtccctgaagctgagctgcgccgcctctgggttcaccttcagcaacttcgccatggcctgggtccgacagcctccgaccaaggggctcgaatgggtcgccagcatcagcacaggcggcggaaatacctactaccgggacagcgtgaaaggccggtttaccatcagccgggacgatgccaagaacacccagtacctccagatggacagcctgcggagcgaagataccgccacgtactattgcgccaggcagcggaactactacgacggcagctacgactacgagggctacaccatggacgcctggggccaagggacctccgtgaccgtcagctcggatcccaccaccaccccagccccacggccacctacccctgccccaaccatcgccagccagcccctgagcctgcggcctgaagcctgcaggcctgccgccggaggagccgtgcacacaaggggcctggacttcgcctgcgacatctatatctgggcccccctggccgggacatgcggggtgctgctgctgtccctggtgattacactgtattgcaaacggggccggaagaagctgctgtacatcttcaagcagcccttcatgcggcccgtgcagaccacccaggaggaggacggctgcagctgccggttccccgaggaagaggaaggcggctgcgagctgcgggtgaagttcagccggagcgccgacgccccagcctaccagcagggccagaaccagctgtacaacgagctgaacctgggacggcgggaggagtacgacgtgctggacaagcggcggggacgggaccccgagatgggcggcaagcctcgccggaagaatccccaggagggcctgtacaacgagctgcagaaggacaagatggccgaggcctacagcgagatcggcatgaagggcgagcggcgccggggcaagggccacgacggcctgtaccagggcctgagcaccgccaccaaggacacctacgacgccctgcacatgcaggccctgccaccccggtga

SEQ ID NO: 91 - protein, 496 aa, synthetic construct
  REGION 1..496: note = Synthetic
METDTLLLWVLLLLVPGSTGDIQMTQSPSSLSASLGDRVTITCRSSQDIGNYLTWFQQKVGRSPRRMIYGAIKLEDGVPSRFSGSRSGSDYSLTISSLESEDVADYQCLQSIQYPFTFGSGTKLEIKRSGGGGSGGGGSGGGGSEVQLVESGGGLVQPGRSLKLSCAASGFTFSNFAMAWVRQPPTKGLEWVASISTGGGNTYYRDSVKGRFTISRDDAKNTQYLQMDSLRSEDTATYYCARQRNYYDGSYDYEGYTMDAWGQGTSVTVSSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR